(19) United States
(12) Patent Application Publication
(10) Pub. No.: US 2022/0056100 A1
(43) Pub. Date: Feb. 24, 2022

(54) COMPOSITIONS AND METHODS FOR USE OF RED-SHIFTED ANION CHANNEL RHODOPSINS

(71) Applicant: BOARD OF REGENTS OF THE UNIVERSITY OF TEXAS SYSTEM, Austin, TX (US)

(72) Inventors: John L. SPUDICH, Houston, TX (US); Oleg A. SINESHCHEKOV, Houston, TX (US); Elena G. GOVORUNOVA, Houston, TX (US)

(73) Assignee: BOARD OF REGENTS OF THE UNIVERSITY OF TEXAS SYSTEM, Austin, TX (US)

(21) Appl. No.: 17/406,670

(22) Filed: Aug. 19, 2021

Related U.S. Application Data
(60) Provisional application No. 63/068,298, filed on Aug. 20, 2020.

(51) Int. Cl. C07K 14/705

(52) U.S. Cl. CPC C07K 14/705; A61K 38/00

(57) ABSTRACT

Methods and compositions used to identify and characterize novel rhodopsin domains, which are anion-conducting channelrhodopsins. The rhodopsin domain of these anion-conducting channelrhodopsins have been cloned, optimized and expressed in mammalian systems and thus may be used in, among others, optogenetic applications and as therapeutic agents for electrically active cell mediated disorders.

### PRIORITY CLAIM

This application claims benefit of priority to U.S. Provisional Application Ser. No. 63/068,298, filed Aug. 20, 2020, the entire contents of which are hereby incorporated by reference.

### STATEMENT REGARDING FEDERALLY SPONSORED RESEARCH

This invention was made with government support under Grant No. R01GM027750 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Aug. 18, 2021, is named UTSHP0369US_ST25.txt and is 27 kilobytes in size.

### BACKGROUND

### 1. Field

The present disclosure relates generally to the fields of molecular biology and medicine. Methods and compositions that use channelrhodopsins, such as anion-conducting channelrhodopsins, are provided. The channelrhodopsins may be used for optogenetic research applications or as therapeutic agents.

### 2. Description of Related Art

Channelrhodopsins are light-gated ion channels, first found in chlorophyte (green) flagellate algae as phototaxis receptors that depolarize the cell membrane by cation conduction (Sineshcheck et al., 2002; Nagel et al., 2020; 2003). The genomes of cryptophyte algae encode a related class of channelrhodopsins that are, however, strictly anion-selective (Govorunova et al., 2015). Both functional classes of channelrhodopsins, known as cation and anion channelrhodopsins (CCRs and ACRs, respectively), are widely used to, respectively, activate (Boyden et al., 2005) and inhibit (Mohammad et al., 2017) neurons with light (optogenetics). Cation conductance appears to have independently evolved also within the family of cryptophyte CCRs, referred to as “bacteriorhodopsin-like” CCRs (BCCRs) because of their sequence homology to archaeal proton-pumping rhodopsins (Sineshchekov et al., 2017). Recently a distinct family of ACRs was identified in environmental DNA samples of unknown organismal origin collected by the Tara Oceans project (Oppermann et al., 2019). This finding raised a possibility that channelrhodopsin genes were not limited to algae and were more widespread among protists.

Long-wavelength light better penetrates biological tissue, and therefore actuator molecules with red-shifted absorption are highly desired for optogenetic applications. A natural CCR variant with peak absorption in the orange-red spectral region has been discovered and used for photostimulation of neuronal firing (Klapoetke et al., 2014). However, this CCR (named Chrimson) could not be converted to an anion channel by mutagenesis (Wietek et al., 2017), and no natural ACRs with an absorption maximum beyond 540 nm had been found so far, despite extensive screening of homologous proteins from various cryptophyte species (Govorunova et al., 2017; 2018).

### SUMMARY

The presently disclosed methods and compositions are based, in part, on the identification and modification of four labyrinthulea ACRs (termed “RubyACRs”) that exhibit maximal spectral sensitivity in the orange-red region of the spectrum, dependent on unique residues of their retinal-binding pocket. Further, energy transfer was observed between a fluorescent protein, fused as a tag to the cytoplasmic C-terminus of a RubyACR, and its photoactive site retinal chromophore, an engineered antenna effect that has not been reported to photoactivate channelrhodopsins.

Sequences encoding rhodopsin domains of Aurantiochytrium limacinum MYA-1381, GenBank accession number MT002467; Aurantiochytrium limacinum MYA-1381, GenBank accession number MT002473; Aurantiochytrium limacinum MYA-1381, GenBank accession number MT002476; Aurantiochytrium sp. KH10S, GenBank accession number MT002468; Hondaea fermentalgiana FCC1311 (Aurantiochytrium sp. FCC1311), GenBank accession number MT002469; Schizochytrium aggregatum ATCC 28209, GenBank accession number MT002463; and Thraustochytrium sp. ATCC 26185, GenBank accession number MT002470, were optimized for human codon usage and were synthesized. Illumination of neurons containing anion-conducting channelrhodopsins are extensively used to inhibit neuronal action potential spikes. These ACRs provide new membrane-hyperpolarizing tools for use in establishing a high level of membrane potential for use as optogenetic tools for neuronal silencing of excited cells for among others, neuronal or neurologic disorders, such as, but not limited to, Parkinson's disease and epilepsy, as well as for cardiac disorders.

In some embodiments herein are disclosed recombinant nucleic acids operatively linked to heterologous promoter sequences, said recombinant nucleic acids comprising: a sequence that encodes a polypeptide with at least 85%, 90%, 91%, 92%, 93%, 94%, 95%, 96%, 97%, 98% or 99% identical to a sequence selected from the group consisting of SEQ ID NO: 4, SEQ ID NO: 8, SEQ ID NO: 12, or SEQ ID NO: 16; or a sequence that encodes a polypeptide comprising at least 225, 230, 235, 240, 255, 260, 265, 270, 275, 280, 285, 290, 295, 300, 305, 310, 315 or 320 contiguous amino acids from SEQ ID NO: 4, SEQ ID NO: 8, SEQ ID NO: 12, or SEQ ID NO: 16, or a sequence that hybridizes to the nucleotide sequence of SEQ ID NO: 1-3, SEQ ID NO: 5-7, SEQ ID NO: 9-11, or SEQ ID NO: 13-15 or the complement thereof. In another embodiment, the recombinant nucleic acids comprise an expression vector. In further embodiments, the recombinant nucleic acids that hybridize to the nucleotide sequence of SEQ ID NO: 1-3, SEQ ID NO: 5-7, SEQ ID NO: 9-11, or SEQ ID NO: 13-15 further hybridize to filter-bound DNA in 0.5 M NaHPO₄, 7% sodium dodecyl sulfate, 1 mM EDTA at 65° C., and washing in 0.2×SSC/0.1% SDS at 42° C.

In a further embodiment, there are provided recombinant host cells comprising a recombinant nucleic acid operatively linked to a heterologous promoter sequence, said recombinant nucleic acids comprising a sequence that encodes a polypeptide with at least 85%, 90%, 91%, 92%, 93%, 94%, 95%, 96%, 97%, 98% or 99% identity to an amino acid sequence selected from SEQ ID NO: 4, SEQ ID NO: 8, SEQ ID NO: 12, or SEQ ID NO: 16 or a sequence that encodes a polypeptide comprising at least 225, 230, 235, 240, 255, 260, 265, 270, 275, 280, 285, 290, 295, 300, 305, 310, 315 or 320 contiguous amino acids from SEQ ID NO: 4, SEQ ID NO: 8, SEQ ID NO: 12, or SEQ ID NO: 16, or a sequence that hybridizes to the nucleotide sequence of SEQ ID NO: 1-3, SEQ ID NO: 5-7, SEQ ID NO: 9-11, or SEQ ID NO: 13-15, or the complement thereof. In some embodiments, the host cell is an isolated human cell, a non-human mammalian cell, a bacterial cell, a yeast cell, an insect cell, or a plant cell.

In some embodiments, there are provided methods of restoring photosensitivity to a retina of a subject suffering from vision loss or blindness, said methods comprising: delivering to the OFF-bipolar neurons of the retina of said subject an expression vector comprising a polynucleotide that encodes: an amino acid sequence at least about 85%, 90%, 91%, 92%, 93%, 94%, 95%, 96%, 97%, 98% or 99% identical to a sequence selected from the group consisting of SEQ ID NO: 4, SEQ ID NO: 8, SEQ ID NO: 12, or SEQ ID NO: 16, which expression vector encodes a humanized rhodopsin domain of a RubyACR expressible in a retinal neuron; and expressing said vector in said retinal neuron, wherein the expressed rhodopsin renders said retinal neuron photosensitive, thereby restoring photosensitivity to enable light-induced silencing of such neuron in said retina or a portion thereof. In a further embodiment, the subject is mammalian, and in a still further embodiment, the subject is human. In some aspects, the method comprises delivering to the retina of said subject an expression vector. In certain aspects, the delivering comprises a pharmaceutically acceptable carrying agent.

In some further embodiments, there are provided isolated nucleic acid molecules comprising a sequence encoding a rhodopsin domain of a RubyACR having a sequence at least about 85%, 90%, 91%, 92%, 93%, 94%, 95%, 96%, 97%, 98% or 99% identical to a sequence according to SEQ ID NO: 1-3, SEQ ID NO: 5-7, SEQ ID NO: 9-11, or SEQ ID NO: 13-15. In certain aspects, the isolated nucleic acid molecule comprises a sequence that hybridized to the nucleotide sequence of one of SEQ ID NO: 1-3, SEQ ID NO: 5-7, SEQ ID NO: 9-11, or SEQ ID NO: 13-15, under stringent conditions comprising hybridization to filter-bound DNA in 0.5 M NaHPO₄, 7% sodium dodecyl sulfate, 1 mM EDTA at 65° C., and washing in 0.2×SSC/0.1% SDS at 42° C. and encodes an anion-conducting channelrhodopsin. In some aspects, the nucleic acid is a DNA. In other aspects, the nucleic acid is an RNA (e.g., mRNA). In further embodiments, there are provided expression vectors comprising a nucleic acid molecule provided herein, such as a sequence at least about 90% identical to a sequence according to SEQ ID NO: 1-3, SEQ ID NO: 5-7, SEQ ID NO: 9-11, or SEQ ID NO: 13-15.

In an even further embodiment, there are provided recombinant host cells comprising a nucleic acid provided herein (e.g., a sequence at least about 90% identical to a sequence according to SEQ ID NO: 1-3, SEQ ID NO: 5-7, SEQ ID NO: 9-11, or SEQ ID NO: 13-15). In some aspects, the host cell is an isolated human cell. In other aspects, the host cell is a non-human mammalian cell. In some aspects, the host cell is a bacterial cell. In certain aspects, the host cell is a yeast cell. In other aspects, the host cell is an insect cell. In some aspects, the host cell is a plant cell. In certain aspects, host cell is an isolated neuronal cell. In particular, the host cell is an isolated electrically active cell.

In another embodiment, there are provided methods of treating a subject suffering from a disorder that involves electrically active cells comprising expressing in the subject an effective amount of a sequence encoding a rhodopsin domain of a RubyACR anion-conducting channelrhodopsin at the site of the electrically active cells. In some aspects, the subject is suffering from neuropathic pain the method comprising expressing in the subject an effective amount of an anion-conducting channelrhodopsin at the site of the pain. In certain aspects, the subject has an amputated limb, diabetes, multiple sclerosis or has undergone a surgery.

In certain aspects, expressing comprises administering an anion-conducting channelrhodopsin to the subject. In some aspects, the anion-conducting channelrhodopsin comprises an amino acid sequence at least about 85%, 90%, 91%, 92%, 93%, 94%, 95%, 96%, 97%, 98% or 99% identical to a sequence of SEQ ID NO: 4, SEQ ID NO: 8, SEQ ID NO: 12, or SEQ ID NO: 16. In certain aspects, the anion-conducting channelrhodopsin is encoded by a sequence about 90%, 91%, 92%, 93%, 94%, 95%, 96%, 97%, 98% or 99% identical to sequence according to SEQ ID NO: 1-3, SEQ ID NO: 5-7, SEQ ID NO: 9-11, or SEQ ID NO: 13-15. In some aspects, the anion-conducting channelrhodopsin further comprises a cell-penetrating peptide (CPP) sequence or a cellular receptor-binding sequence. As used herein the terms “cell penetrating peptide” refers to segments of polypeptide sequence that allow a polypeptide to cross the cell membrane (e.g., the plasma membrane in the case a eukaryotic cell). Examples of CPP segments include, but are not limited to, segments derived from HIV Tat (e.g., GRKKRRQRRRPPQ (SEQ ID NO: 17)), herpes virus VP22, the Drosophila Antennapedia homeobox gene product, protegrin I, Penetratin (RQIKIWFQNRRMKWKK (SEQ ID NO: 23)) or melittin (GIGAVLKVLTTGLPALISWIKRKRQQ (SEQ ID NO: 18)). In certain aspects the CPP comprises the T1 (TKIESLKEHG (SEQ ID NO: 19)), T2 (TQIENLKEKG (SEQ ID NO: 20)), 26 (AALEALAEALEALAEALEALAEAAAA (SEQ ID NO: 21)) or INF7 (GLFEAIEGFIENGWEGMIEGWYGCG (SEQ ID NO: 22)) CPP sequence.

In some aspects, expressing comprises administering a vector encoding an anion-conducting channelrhodopsin to the subject encodes a rhodopsin domain of a RubyACR. In certain aspects, the vector is an RNA vector. In other aspects, the vector is a DNA vector. In some aspects, the vector is a plasmid, a viral vector or an episomal vector. In certain aspects, the vector further comprises an inducible expression cassette for a suicide gene.

In certain aspects, the sequence encoding the anion-conducting channelrhodopsin encodes a rhodopsin domain of a RubyACR is operably linked to a heterologous promoter. In some aspects, the promoter is an inducible or a repressible promoter. In certain aspects, the promoter is a tissue or cell type specific promoter. In particular, the promoter is neuronal cell specific promoter.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-F. (FIG. 1A) A phylogenetic tree of rhodopsin domains constructed by the neighbor joining method. The ultrafast bootstrap support values are shown by red circles (95-100% range). The GenBank accession numbers and source organism names of the sequences used are listed in Tables 2 and 3. (FIG. 1B) The action spectra of photocurrents generated by indicated ACRs. The data points are the mean values±sem (n=6-10 scans). (FIG. 1C) The peak current amplitudes (top) and spectral maxima (bottom) of all functional ACRs tested in this study except AlACR2, generated in response to the first 1-s light pulse after seal formation. The data from individual cells are shown as circles, the lines show the mean values±sem (n=7-13 cells). (FIG. 1D and FIG. 1E) Series of photocurrents recorded using the indicated Cl⁻ concentrations (in mM) at voltages changed from −60 to 60 mV at the amplifier output. (FIG. 1F) The reversal potentials (Eᵣₑᵥ) of photocurrents measured under indicated ionic conditions (in mM). The data from individual cells are shown as circles, the lines show the mean values±sem (n=5 cells). The Eᵣ, values were corrected for liquid junction potentials, as described in Methods.

FIGS. 2A-I. (FIG. 2A) The residues in the retinal-binding pocket of indicated proteins. The numbers correspond to the sequence of AlACR1. BR, bacteriorhodopsin. (FIG. 2B) A homology model of AlACR1 showing the side chains of the residues from panel A (yellow with red labels) and the chromophore and corresponding side chains of GtACR1 (green; PDB entry code: 6EDQ). PSB+, protonated Schiff base. (FIGS. 2C-H) The action spectra of photocurrents generated by the indicated mutants of AlACR1 (red) and that of the wild type from FIG. 1B (black). (FIG. 2I) The dependence of contribution of the 520-nm peak (its ratio to the rhodopsin peak) on the position of the rhodopsin peak.

FIGS. 3A-D. (FIG. 3A) The action spectra of photocurrents generated by AlACR1 fused to EYFP or mCherry and the absorption spectrum of fluorescence tag-free AlACR1 purified from Pichia. (FIG. 3B) The difference between the action spectra obtained with AlACR1 EYFP and AlACR1 mCherry and the fluorescence excitation spectrum of EYFP from FPbase (available on the world wide web at fpbase.org/protein/eyfp/). (FIG. 3C and FIG. 3D) The action spectra of photocurrents recorded by AlACR2 (FIG. 3C) and AlACR3 (D) EYFP fusions (black dashed lines, reproduced from FIG. 1B) and the absorption spectra of the respective purified proteins (solid red lines).

FIGS. 4A-F. (FIG. 4A) A series of photocurrent traces recorded from AlACR1 upon laser flash excitation at the voltages changed in 20 mV increment from −60 to 60 mV at the amplifier output. Black lines, experimental data; red lines, multiexponential fit. (FIG. 4B and FIG. 4C) The dependence of the decay components τ (FIG. 4B) and amplitude (FIG. 4C) on the holding voltage for the series of traces shown in FIG. 4A. (FIG. 4D) A series of photocurrent traces recorded from the AlACR1_T72E mutant at incremental voltages from −60 to 60 mV. The wild-type (WT) trace at −60 mV from FIG. 4A is shown as the dashed line for comparison. (FIG. 4E and FIG. 4F) The current traces recorded from the indicated AlACR1 mutants at −60 mV (solid lines) as compared to the wild-type trace (dashed lines).

FIGS. 5A-D. (FIG. 5A) A photocurrent trace recorded from AlACR2 upon stimulation with a continuous light pulse, the duration of which is schematically shown as the green bar. (FIG. 5B) A series of photocurrent traces recorded from AlACR2 upon laser flash excitation at the voltages changed from −60 to 60 mV at the amplifier output with the standard pipette solution. (FIG. 5C) Main figure: A photocurrent trace recorded from AlACR2 at 60 mV using the pipette solution with a reduced Cl⁻ concentration. Inset: a portion of the trace from 5 to 100 ms vertically stretched to better resolve the channel current decay. Black lines, experimental data; red lines, multiexponential fit. (FIG. 5D) The current-voltage dependencies of the peak current and the slowest signal component at the indicated Cl⁻ concentrations in the pipette. The data points are the mean±sem values (n=5 cells).

FIG. 6. Protein sequence alignment of the rhodopsin domains of labyrinthulea channelrhodopsins. Residues are color-coded according to their chemical properties. The red rectangles show α-helical regions as detected in the AlACR1 homology model. The arrows point to the positions of the residues known to be functionally important in GtACR1 (GtACR1 numbering).

FIG. 7. Protein sequence alignment of the rhodopsin domains of haptophyte channelrhodopsins. Residues are color-coded according to their chemical properties. The red rectangles show α-helical regions as detected in the PgACR1 homology model. The arrows point to the positions of the residues known to be functionally important in GtACR1 (GtACR1 numbering).

FIG. 8. An unrooted phylogenetic tree of labyrinthulea rhodopsin domains. The ultrafast bootstrap support values are shown by red circles (95-100% range). The colored leaf labels show proteins tested in this study; the color of the label corresponds to the wavelength of the maximal sensitivity.

FIGS. 9A-D. (FIG. 9A) An example of photocurrent traces generated by SaACR2 at two different wavelengths (black lines), linear approximation of their initial segments (blue lines) and slopes (α1 and α2) used for construction of the action spectra. (FIGS. 9B-D) The action spectra of the indicated homologs, constructed as shown in panel A. The data points are the mean values±sem (n=6-8 scans).

FIG. 10. An alignment of AlACR1 and HfACR1 rhodopsin domains. Identical residues are shaded black.

FIG. 11. Photocurrent traces recorded in response to the first 1-s light pulse at −60 mV at the amplifier output, normalized at their peak value. The duration of illumination is showed as a colored bar on top.

FIGS. 12A-D. (FIG. 12A) Stationary current measured at the end of a 1-s light pulse. (FIG. 12B) The magnitude of desensitization during 1-s continuous illumination. (FIG. 12C) Half-decay of photocurrent after switching the light off. In A-C, the black lines show the mean values and s.e.m. (n=5-10 cells for each variant); colored circles, the individual data points. (FIG. 12D) The ratio of the peak amplitude to that of the stationary current measured in a series of 1-s pulses applied with 30-s time interval. The lines are single exponential fits.

FIGS. 13A-B. Performance of HfACR1 activated at 630 nm (5.6 mW·cm-2) and tested under ionic conditions typical of mature neurons (for solution compositions see Methods). (FIG. 13A) A current-voltage relationship. The data points are the mean±sem (n=5 cells). (FIG. 13B) Photocurrent traces recorded from HfACR1 upon repetitive stimulation with 2-ms light pulses at −20 mV at the amplifier output.

FIG. 14. Peak (red) and stationary (blue) photocurrents recorded from color-tuning mutations of AlACR1. The mean±sem are shown as lines and whiskers (n=7), data from individual cells, as diamonds. The data for wild type (WT) are taken from FIG. 1C. Triple, the F108V_Y171I_I217P mutant.

FIGS. 15A-D. (FIG. 15A and FIG. 15B) The action spectra of photocurrents generated by indicated proteins reconstituted with A1 (black) and A2 (red) retinal. (FIG. 15C) The difference spectra (A2-A1 retinal). (FIG. 15D) The absorption spectrum of HfACR1 detergent-purified from Pichia (red solid line) compared to the action spectrum of photocurrents generated upon its expression in HEK293 cells from FIG. 1B (black dashed line).

FIG. 16. The action spectrum of photocurrents generated by AlACR2 reconstituted with A1 (black) and A2 (red) retinal.

### DETAILED DESCRIPTION

Provided herein are two distinct ACR families from labyrinthulea and haptophytes and their modification. Labyrinthulea (also known as Labyrinthomycetes) are a class of aquatic heterotrophic microbes. Despite the lack of flagella in vegetative stages, the peculiar lifestyles and the presence of the bothrosome in many members, the labyrinthulea are typical stramenopiles as evidenced by their heterokont zoospores (when present). Labyrinthulomycete ancestors are believed to have never been photosynthetic (Leyland et al., 2017), unlike some other heterotrophic eukaryotic lineages that evolved by plastid loss. Haptophytes are flagellate algae distantly related to cryptophytes (Reeb et al., 2009; Burki et al., 2020).

Channelrhodopsins are light-gated ion channels widely used to control neuronal firing with light (optogenetics). Two previously unknown families of anion channelrhodopsins (ACRs) are provided, one from the heterotrophic protists labyrinthulea and the other from haptophyte algae. Four closely related labyrinthulea ACRs, named RubyACRs here, exhibit a unique retinal-binding pocket that creates spectral sensitivities with maxima at 590-610 nm, the most red-shifted channelrhodopsins known, long-sought for optogenetics, and more broadly the most red-shifted microbial rhodopsins so far reported. RubyACRs are provided for use as optogenetics tools for research and clinical applications to enable light-control of neuron electrical activity and other processes regulated by chloride and other halide electrochemical potential. RubyACR properties enable photocontrol of neurons at deep tissue levels eliminating the need for tissue-invasive light pipes and enabling lower expression levels of the channels.

Three spectral tuning residues were identified as critical for the red-shifted absorption. Photocurrents recorded from the RubyACR from Aurantiochytrium limacinum (designated AlACR1) under single-turnover excitation exhibited biphasic decay, the rate of which was only weakly voltage-dependent, in contrast to that in previously characterized cryptophyte ACRs, indicating differences in channel gating mechanisms between the two ACR families. Moreover, in A. limacinum three ACRs were identified with absorption maxima at 485, 545, and 590 nm, indicating color-sensitive photosensing with blue, green and red spectral variation of ACRs within individual species of the labyrinthulea family. Functional energy transfer from a cytoplasmic fluorescent protein domain to the retinal chromophore bound within RubyACRs is also provided.

The identification and characterization of two ACR families, one from non-photosynthetic microorganisms, shows that light-gated anion conductance is more widely spread among eukaryotic lineages than previously thought. The strongly red-shifted absorption spectra of the subset designated RubyACRs make them promising candidates for the long-sought inhibitory optogenetic tools producing large passive currents activated by red light, enabling deep tissue penetration. Previously only low-efficiency ion-pumping rhodopsins were available for neural inhibition at similar long wavelengths. The unusual residue composition of the retinal-binding pocket in RubyACRs expands the understanding of color tuning in rhodopsins. Finally, activation of chloride currents by energy transfer from a cytoplasmic fluorescent tag on RubyACRs opens a potential new dimension in molecular engineering of optogenetic tools.

Green-light and blue-light driven ACRs are used as neuron inhibitors, but only RubyACRs can be photocontrolled in moderate to deep tissue because of the effective tissue-penetration of longer wavelength (orange and red) light, because of low light-scattering and low absorption by tissue components. Light-driven ion pumps, some with longer wavelength absorption spectra than the green and blue-sensitive rhodopsins, have also been used as optogenetics tools for inhibiting neuron firing, but their low ion translocation conductivity is orders of magnitude less effective in controlling cell electrochemical potential than ACR channels.

### I. RUBYACR

Provided herein are channelrhodopsins from two eukaryotic lineages phylogenetically distant from chlorophyte and cryptophyte algae in which such proteins had so far been found. Both labyrinthulea and haptophyte ACRs conduct exclusively anions and therefore should be classified as ACRs. This expansion of the ACR family benefits elucidation of the structural requirements for anion conductance by comparative analysis of the structure-function relationships in different ACR groups and offers a possibility of using alternative scaffolds for molecular design of physico-chemical properties desirable in optogenetic tools. The most important in this respect are the four RubyACRs, closely related labyrinthulea ACRs with spectral maxima in the orange-red region. The only so far known inhibitory optogenetic tool with the peak absorption at ˜590 nm has been the engineered halorhodopsin from H. salinarum strain Shark, referred to as Jaws (Chuong et al., 2014). Although Jaws generates larger photocurrents than the earlier known Cl⁻ pumps, its efficiency is limited by translocation of only one ion per captured photon across the membrane. RubyACRs generate large passive anion currents and offer a new type of highly efficient optogenetic inhibition for the long-wavelength spectral range.

The analysis of spectral-tuning mutants and A1/A2 retinal substitution strongly indicate Førster resonance energy transfer (FRET) from a fluorescent protein (EYFP) to the retinal chromophore in red-shifted rhodopsins, which may provide an additional avenue for the development of optogenetic tools. Previously, energy transfer from a light-harvesting antenna (the carotenoid salinixanthin) to the retinal chromophore was reported in xanthorhodopsin, a eubacterial proton pump (Balashov et al., 2007). This phenomenon has also been demonstrated in the heterologously expressed proton-pumping rhodopsin from the cyanobacterium Gloeobacter violaceous reconstituted with the carotenoid echinenone present in this organism (Balashov et al., 2010). In the crystal structure of xanthorhodopsin, the ring of salinixanthin is bound 5 Å from the retinal chromophore's β-ionone ring (Luecke et al., 2008). Salinixanthin binding depends on the presence of retinal, but does not require formation of the Schiff base linkage (Imasheva et al., 2008). Fluorescence spectroscopy and femtosecond transient absorption spectroscopy have shown that the energy transfer mostly occurs from the excited singlet (S2) state of salinixanthin to the 51 state of the retinal chromophore (Polivka et al., 2009).

The molecular system in which the energy transfer was observed in this study differs in that the two chromophores occur within two different protein domains: water-soluble EYFP and membrane-embedded rhodopsin. FRET in similar complexes between fluorescent proteins and rhodopsins has been reported (Gong et al., 2014; Zou et al., 2014; Azimi Hashemi et al., 2019). In those studies, quenching of or increase in the donor fluorescence was used to monitor the state of the acceptor (a voltage reporter). Instead, the present observations show “functional FRET”.

The influence of retinal-binding pocket residues on the absorption spectra (color tuning) in rhodopsins has been extensively studied by experimental and computational approaches (Fujimoto et al., 2007; Melaccio et al., 2012; Katayama et al., 2015; Gozem et al., 2017). However, the residue pattern conserved in RubyACRs has not been found in any other microbial rhodopsins, including the cation-conducting Chrimson that exhibits peak absorption at 590 nm. Of the three pocket residues that contribute to the red-shifted spectral sensitivity of RubyACRs, Phe108 (AlACR1 numbering) occupies the position of the counterion of the Schiff base (Asp85 in bacteriorhodopsin), the importance of which for color tuning is well documented (Fujimoto et al., 2007; Hufen et al., 2004; Karasuyama et al., 2018). As shown in bacteriorhodopsin, a neutral residue in this position destabilizes the ground state decreasing the energy gap between the ground and excited states as evidenced by red-shifted absorption (Marti et al., 1991). A bulky aromatic residue in the counterion position is very unusual among microbial rhodopsins. A previously known group of sequences in which phenylalanine was found at this position are schizorhodopsins from Asgardarchaeota (Inoue et al., 20202) and some of their bacterial relatives, Antarctic rhodopsins (Harris et al., 2020), but they show very little homology to channelrhodopsins and act as inward proton pumps. A possible role of this substitution in color tuning in these proteins has not been tested.

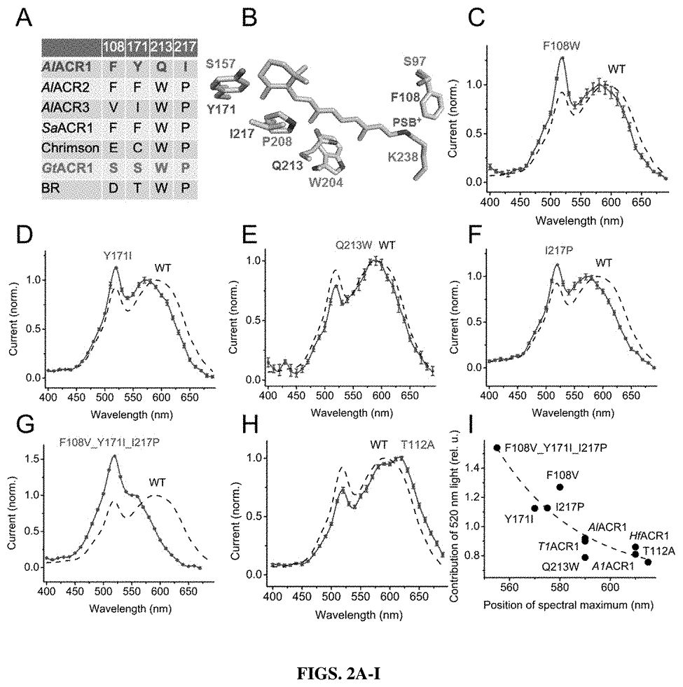

According to the homology modeling of AlACR1, Tyr171 and Ile217 are located near the β-ionone ring, as shown overlaid with the retinal binding pocket of GtACR1 (FIG. 2B), the only ACR crystal structure available. The red-shifting effect of polar Tyr171 on the AlACR1 spectrum is likely due to stabilization of the excited state of the chromophore, which would occur if the dipole moment associated with the side chain is oriented with its negative pole toward the retinal chromophore (Melaccio et al., 2012). The orientation of the Tyr171 side chain needs to be resolved by atomic structure determination. Ile217 of AlACR1 corresponds to Pro219 of the sodium-pumping rhodopsin KR2. Replacement of this residue with non-polar Gly caused a 10-nm red spectral shift in KR2, which was explained by the influence of this mutation on the dipole created by the C═O and N—H groups of the backbone (Inoue et al., 2019). A similar explanation may be suggested for Ile217, but again needs structural verification and computational analysis.

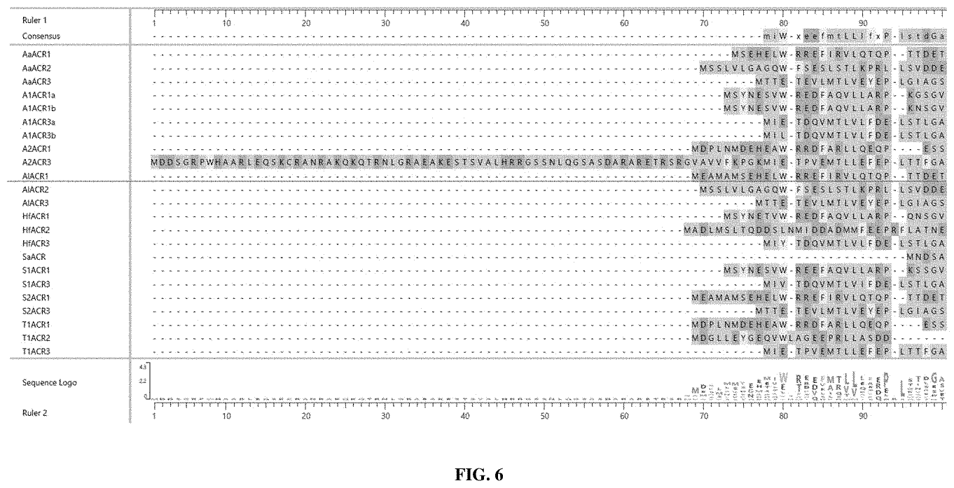

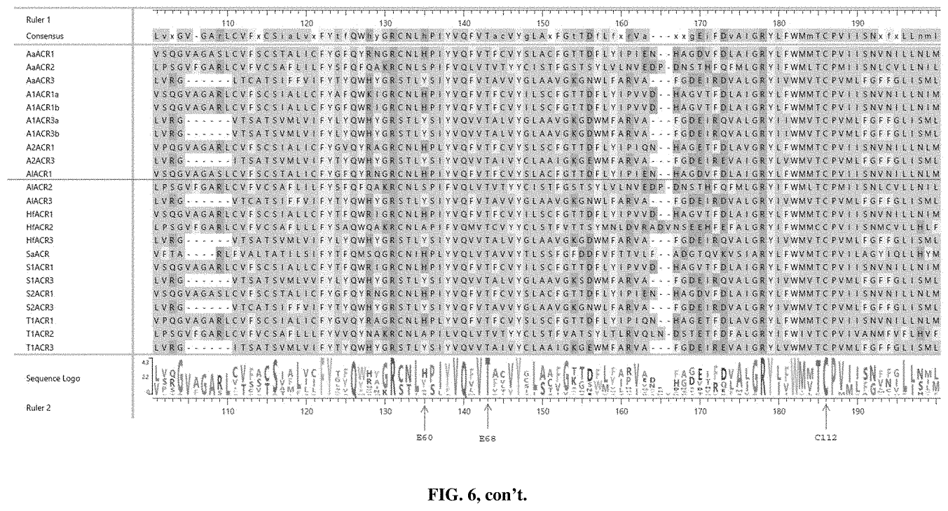

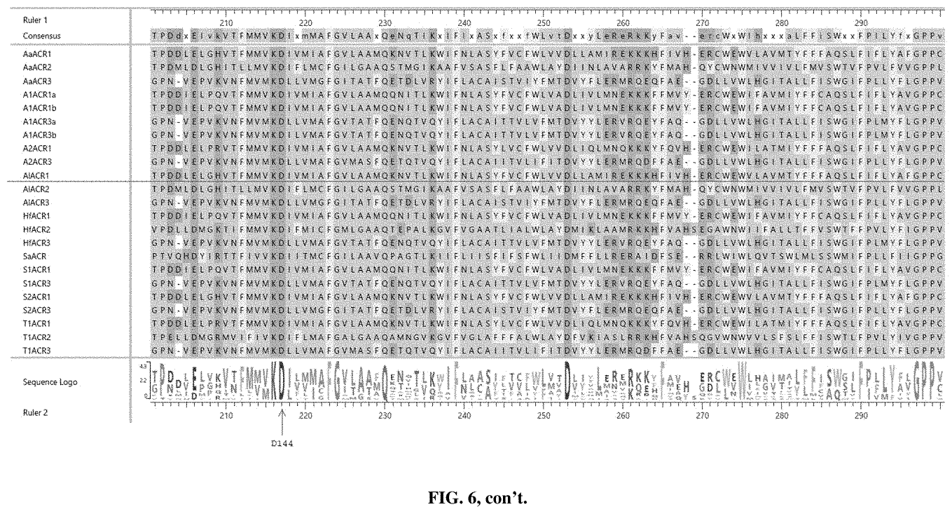

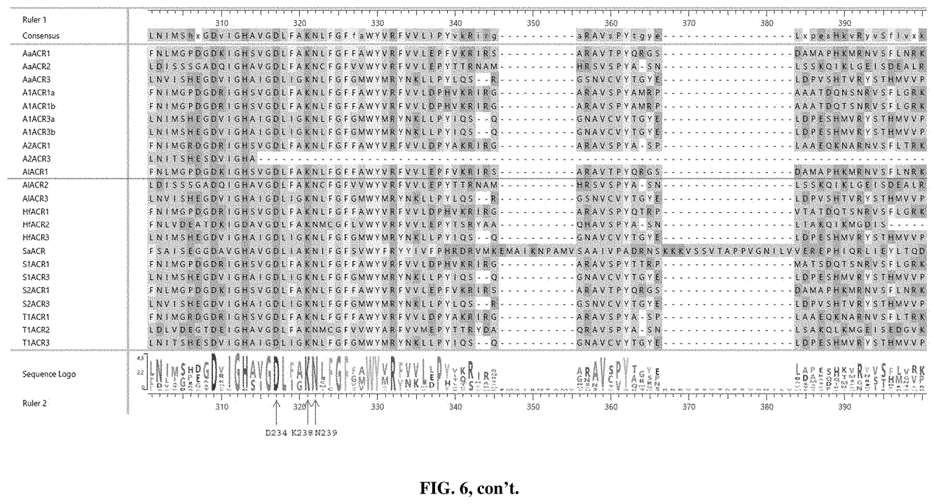

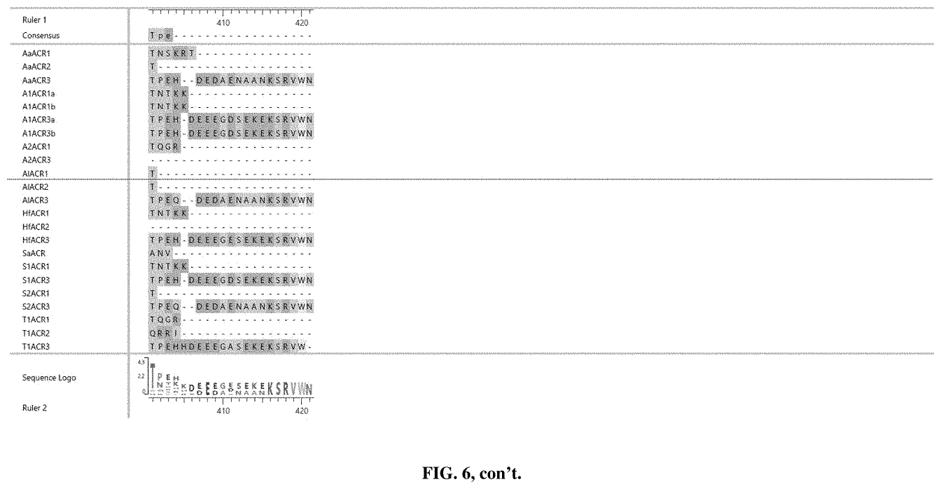

The combination of the three color-tuning mutations, F108V, Y171I and I217P, did not shift the spectral maximum of AlACR1 all the way to that of AlACR3 (FIG. 2G), indicating that there are other residues that contribute to the spectral difference. One candidate is the residue corresponding to Leu93 in bacteriorhodopsin. This position has been shown to determine the spectral difference between green- and blue-absorbing proteorhodopsins (Man et al., 2003). All RubyACRs have Ile in this position (Ile116 in AlACR1) as do green-absorbing AlACR2 and SaACR2, whereas the blue-absorbing AlACR3 has Met (FIG. 6).

The only species in which the functional role of channelrhodopsins has been demonstrated is the green flagellate alga Chlamydomonas reinhardtii, in which they function as phototaxis receptors (Sineshchekov et al., 2002). All labyrinthulea and haptophyte species in which channelrhodopsins were found produce free-swimming zoospores in their lifecycles (Peperzak et al., 2000; Bennett et al., 2017). Therefore, it is plausible that channelrhodopsins guide phototaxis also in these microorganisms. Consistent with this hypothesis is that no channelrhodopsin homologs have been found in the genome of the labyrinthulea Aplanochytrium kerguelense PBS07, which does not produce zoospores (Bennett et al., 2017). However, two paralogs have been found in the transcriptome of A. stocchinoi (Rozenberg et al., 2020), although there are no known flagellate stages in its lifecycle (Moro et al., 2003).

### II. CHANNELRHODOPSINS

Sequences encoding RubyACRs are provided herein. In particular, previously unknown channelrhodopsins having the amino acid sequence of SEQ ID NO: 4, SEQ ID NO: 8, SEQ ID NO: 12, or SEQ ID NO: 16 or encoded by the polynucleotide sequence of SEQ ID NO: 1-3, SEQ ID NO: 5-7, SEQ ID NO: 9-11, or SEQ ID NO: 13-15 are provided.

In some embodiments, are conserved variants of functional humanized rhodopsin domain or a peptide fragment thereof. A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues which are typically incorporated by chemical peptide synthesis rather than by synthesis in biological systems. These include, but are not limited to, peptidomimetics and other reversed or inverted forms of amino acid moieties.

In some embodiments, are any of the disclosed methods, wherein the rhodopsin domain of an anion-conducting channelrhodopsin having the amino acid sequence of all or part of SEQ ID NO: 4, SEQ ID NO: 8, SEQ ID NO: 12, or SEQ ID NO: 16 or a biologically active fragment thereof that retains the biological activity of the encoded rhodopsin domain of an anion-conducting channelrhodopsin or a biologically active conservative amino acid substitution variant of SEQ ID NO: 4, SEQ ID NO: 8, SEQ ID NO: 12, or SEQ ID NO: 16 or of said fragment.

A. Channelrhodopsin Polypeptides

In some embodiments, are isolated polypeptides that encode a rhodopsin domain of an anion-conducting channelrhodopsin. In some embodiments, an isolated polypeptide comprising the amino acid sequence of SEQ ID NO: 4, SEQ ID NO: 8, SEQ ID NO: 12, or SEQ ID NO: 16. In some embodiments, the isolated polypeptide has at least 85% homology to the amino acid sequence of SEQ ID NO: 4, SEQ ID NO: 8, SEQ ID NO: 12, or SEQ ID NO: 16. In some embodiments, the isolated polypeptide has between 85%-95%-100% homology to the amino acid sequence of SEQ ID NO: 4, SEQ ID NO: 8, SEQ ID NO: 12, or SEQ ID NO: 16.

In some embodiments, is a protein composition comprises a polypeptide having the amino acid sequence of SEQ ID NO: 4, SEQ ID NO: 8, SEQ ID NO: 12, or SEQ ID NO: 16. The peptide amino acid sequences that can be used in various embodiments including the anion-conducting rhodopsin domain amino acid sequences described herein (SEQ ID NO: 4, SEQ ID NO: 8, SEQ ID NO: 12, or SEQ ID NO: 16), as well as analogues and derivatives thereof and functional fragments such as but not limited to the rhodopsin/7TM domain. In fact, in some embodiments the any desired peptide amino acid sequences encoded by particular nucleotide sequences can be used, as is the use of any polynucleotide sequences encoding all, or any portion, of desired peptide amino acid sequences. The degenerate nature of the genetic code is well-known, and, accordingly, each anion-conducting rhodopsin domain peptide amino acid-encoding nucleotide sequence is generically representative of the well-known nucleic acid “triplet” codon, or in many cases codons, that can encode the amino acid. As such, as contemplated herein, the anion-conducting channelrhodopsin peptide amino acid sequences described herein, when taken together with the genetic code (see, e.g., “Molecular Cell Biology”, Table 4-1 at page 109 (Darnell et al., eds., W. H. Freeman & Company, New York, N.Y., 1986)), are generically representative of all the various permutations and combinations of nucleic acid sequences that can encode such amino acid sequences.

Such functionally equivalent peptide amino acid sequences (conservative substitutions) include, but are not limited to, additions or substitutions of amino acid residues within the amino acid sequences encoded by a nucleotide sequence, but that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Naturally occurring residues may be divided into classes based on common side chain properties: hydrophobic (Met, Ala, Val, Leu, Ile); neutral hydrophilic (Cys, Ser, Thr, Asn, Gln); acidic (Asp, Glu); basic (His, Lys, Arg); residues that influence chain orientation (Gly, Pro); and aromatic (Trp, Tyr, Phe). For example, non-conservative substitutions may involve the exchange of a member of one of these classes for a member from another class.

In making substitutions, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/Cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, in certain instances, is understood in the art (Kyte et al., J. Mol. Biol., 157:105-131 (1982)). It is known that in certain instances, certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

Substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included.

A skilled artisan will be able to determine suitable variants of a polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides.

In certain embodiments, even areas that may be important for biological activity or for structure may be subject to conservative amino acid substitutions without destroying the biological activity or without adversely affecting the polypeptide structure.

Additionally, in certain embodiments, one skilled in the art can review structure-function studies identifying residues in similar polypeptides that are important for activity or structure. In view of such a comparison, in certain embodiments, one can predict the importance of amino acid residues in a protein that correspond to amino acid residues which are important for activity or structure in similar proteins. In certain embodiments, one skilled in the art may opt for chemically similar amino acid substitutions for such predicted important amino acid residues.

In certain embodiments, one skilled in the art can also analyze the three-dimensional structure and amino acid sequence in relation to that structure in similar polypeptides. In certain embodiments, in view of such information, one skilled in the art may predict the alignment of amino acid residues of a polypeptide with respect to its three dimensional structure. In certain embodiments, one skilled in the art may choose not to make radical changes to amino acid residues predicted to be on the surface of the protein, since such residues may be involved in important interactions with other molecules.

Moreover, in certain embodiments, one skilled in the art may generate test variants containing a single amino acid substitution at each desired amino acid residue. In certain embodiments, the variants can then be screened using activity assays known to those skilled in the art. In certain embodiments, such variants could be used to gather information about suitable variants. For example, in certain embodiments, if one discovered that a change to a particular amino acid residue resulted in destroyed, undesirably reduced, or unsuitable activity, variants with such a change may be avoided. In other words, in certain embodiments, based on information gathered from such routine experiments, one skilled in the art can readily determine the amino acids where further substitutions should be avoided either alone or in combination with other mutations.

A number of scientific publications have been devoted to the prediction of secondary structure. See, e.g., Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The growth of the protein structural database (PDB) has provided enhanced predictability of secondary structure, including the potential number of folds within a polypeptide's structure. See, e.g., Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (see, e.g., Jones, D., Curr. Opin. Struct. Biol., 7(3):377-87 (1997); Sippl et al., Structure, 4(1):15-19 (1996)), “profile analysis” (see, e.g., Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (see, e.g., Holm et al., Nucl. Acid. Res., 27(1):244-247 (1999), and Brenner et al., Curr. Op. Struct. Biol., 7(3):369-376 (1997)).

In certain embodiments, a variant of the reference channelrhodopsin or rhodopsin domain, such as those encoded by SEQ ID NO: 4, SEQ ID NO: 8, SEQ ID NO: 12, or SEQ ID NO: 16 includes a glycosylation variant wherein the number and/or type of glycosylation sites have been altered relative to the amino acid sequence of the reference anion-conducting channelrhodopsin or rhodopsin domain. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created. Exemplary variants include cysteine variants wherein one or more cysteine residues are deleted from or substituted for another amino acid (e.g., serine) relative to the amino acid sequence of the reference channelrhodopsin or rhodopsin domain (e.g., such as those provided as SEQ ID NO: 4, SEQ ID NO: 8, SEQ ID NO: 12, or SEQ ID NO: 16). In certain embodiments, cysteine variants may be useful when polypeptides and proteins must be refolded into a biologically active conformation such as after the isolation of insoluble inclusion bodies. In certain embodiments, cysteine variants have fewer cysteine residues than the native polypeptide. In certain embodiments, cysteine variants have an even number of cysteine residues to minimize interactions resulting from unpaired cysteines.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in a naturally occurring sequence (in certain embodiments, in the portion of the polypeptide outside the domain(s) forming intermolecular contacts). In certain embodiments, a conservative amino acid substitution typically may not substantially change the structural characteristics of the reference sequence (e.g., in certain embodiments, a replacement amino acid should not tend to break a helix that occurs in the reference sequence, or disrupt other types of secondary structure that characterizes the reference sequence).

Examples of certain art-recognized polypeptide secondary and tertiary structures are described, for example, in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

In other embodiments, are methods and compositions that provide an ACR with improved properties and characteristics that enhance the application of the compositions in, among other things, optogenetic techniques. Improved properties include, but are not limited tom adaptation to human codon usage and synthesis. These embodiments provide greater sensitivity and efficient membrane hyperpolarization and neuronal silencing through light-gated chloride conduction or in the case of neurons with high cytoplasmic chloride concentration, wherein the expressed rhodopsin depolarizes the plasma membrane.

B. Fusion Proteins

The use of fusion proteins in which a polypeptide or peptide, or a truncated or mutant version of peptide is fused to an unrelated or homologous protein, polypeptide, or peptide, and can be designed on the basis of the desired peptide encoding nucleic acid and/or amino acid sequences described herein. Such fusion proteins include, but are not limited to: IgFc fusions, which stabilize proteins or peptides and prolong half-life in vivo; fusions to any amino acid sequence that allows the fusion protein to be anchored to the cell membrane; or fusions to an enzyme, fluorescent protein, or luminescent protein that provides a marker function. Exemplary fluorescent proteins include green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyan1, Midori-Ishi Cyan, TagCFP, mTFP1, enhanced yellow fluorescent protein (EYFP), Topaz, MCitrine, YPet, TagYFP, PhiYFP, ZsYellow 1, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRed1, mRaspberry, dKeima-Tandem, mPlum, or AQ143.

Fusion proteins to homologous proteins include, but are not limited to, those that are produced from genes that are engineered to encode a portion of the anion-conducting channelrhodopsin fused to a portion of a homologous (orthologous or paralogous) protein of the same of related function. For example, chimeras between different channelrhodopsins may be made to combine beneficial properties uniquely present in each. In some aspects, a chimeric channelrhodopsin of the embodiments comprises about 50%, 55%, 60%, 65%, 70%, 75%, 80%, 85%, 90% or 95% of its sequence from a first channelrhodopsin and the remaining sequence from a second channelrhodopsin. In some aspects, a chimeric channelrhodopsin comprises the rhodopsin domain of a first channelrhodopsin and the remaining sequence from a second channelrhodopsin. In yet further aspects, a chimeric channelrhodopsin can comprise 1, 2, 3, 4, 5 or 6 of its transmembrane domains from a first channelrhodopsin and the remaining transmembrane domains from a second channelrhodopsin.

In certain embodiments, a fusion protein may be readily purified by utilizing an antibody that selectively binds to the fusion protein being expressed. In alternate embodiments, a fusion protein may be purified by subcloning peptide encoding nucleic acid sequence into a recombination plasmid, or a portion thereof, is translationally fused to an amino-terminal (N-terminal) or carboxy-terminal (C-terminal) tag consisting of six histidine residues (a “His-tag”; see, e.g., Janknecht et al., Proc. Natl. Acad. Sci. USA 88:8972-8976, 1991). Extracts from cells expressing such a construct are loaded onto Ni²⁺ nitriloacetic acid-agarose columns, and histidine-tagged proteins are selectively eluted with imidazole-containing buffers.

C. Nucleic Acids Encoding Channelrhodopsins

In some embodiments, a recombinant nucleic acid operatively linked to a heterologous promoter sequence, said recombinant nucleic acid comprising: a sequence that encodes a peptide with at least 85% homology to an amino acid sequence selected from SEQ ID NO: 4, SEQ ID NO: 8, SEQ ID NO: 12, or SEQ ID NO: 16 or a sequence that encodes a peptide comprising 225 contiguous amino acids selected from SEQ ID NO: 4, SEQ ID NO: 8, SEQ ID NO: 12, or SEQ ID NO: 16; or a sequence that hybridizes to the nucleotide sequence of SEQ ID NO: 1-3, SEQ ID NO: 5-7, SEQ ID NO: 9-11, or SEQ ID NO: 13-15 or the complement thereof.

In some embodiments, are isolated nucleic acid molecules comprising a nucleotide sequence that was derived from cDNA and encode the rhodopsin domain of a RubyACR. In some embodiments, the rhodopsin domain encodes the peptides whose sequence is described in SEQ ID NO: 4, SEQ ID NO: 8, SEQ ID NO: 12, or SEQ ID NO: 16. In some embodiments, are isolated nucleic acid molecules that were derived from cDNA that comprise a nucleotide sequence that encodes the amino acid sequence shown in SEQ ID NO: 4, SEQ ID NO: 8, SEQ ID NO: 12, or SEQ ID NO: 16. In some embodiments, are expression vectors comprising a nucleic acid sequence that encodes the amino acid sequences of SEQ ID NO: 4, SEQ ID NO: 8, SEQ ID NO: 12, or SEQ ID NO: 16. In some embodiments, are host cells comprising a recombinant expression vector comprising a nucleic acid sequence that was derived from cDNA and encode the amino acid sequences of SEQ ID NO: 4, SEQ ID NO: 8, SEQ ID NO: 12, or SEQ ID NO: 16.

In some embodiments are isolated peptides comprising an amino acid sequence encoded by at least a portion of the cDNA derived nucleic acid sequences that encode the 7TM or rhodopsin domain of an ACR. In some embodiments, are isolated peptides comprising an amino acid sequence encoded by a cDNA derived nucleic acid sequence that encodes an amino acid sequence of SEQ ID NO: 4, SEQ ID NO: 8, SEQ ID NO: 12, or SEQ ID NO: 16.

In some embodiments are isolated peptides comprising a contiguous sequence encoded by a nucleic acid sequence that encodes the anion rhodopsin domain of an anion-conducting channelrhodopsin derived. In some embodiments, are isolated peptides comprising an amino acid sequence of SEQ ID NO: 4, SEQ ID NO: 8, SEQ ID NO: 12, or SEQ ID NO: 16, or fragment thereof. In some embodiments, are isolated peptides comprising an amino acid sequence encoded by at least a portion of a nucleic acid sequence of a group consisting of SEQ ID NO: 1-3, SEQ ID NO: 5-7, SEQ ID NO: 9-11, or SEQ ID NO: 13-15 and which functions as a anion rhodopsin or anion-conducting channelrhodopsin.

In some embodiments, are isolated peptides comprising an amino acid sequence of SEQ ID NO: 4, SEQ ID NO: 8, SEQ ID NO: 12, or SEQ ID NO: 16 or a 7 TM domain/rhodopsin domain encoded by a cDNA derived nucleic acid sequence of SEQ ID NO: 1-3, SEQ ID NO: 5-7, SEQ ID NO: 9-11, or SEQ ID NO: 13-15 and which functions as an anion-conducting channelrhodopsin.

In some embodiments, isolated nucleic acid molecules are provided comprising a nucleotide sequence that encodes the rhodopsin domain of an anion-conducting channelrhodopsin. In some embodiments, the rhodopsin encodes a peptide whose sequence is shown in SEQ ID NO: 4, SEQ ID NO: 8, SEQ ID NO: 12, or SEQ ID NO: 16. In some embodiments, are isolated nucleic acid molecules comprising a nucleotide sequence that encodes the amino acid sequence shown in SEQ ID NO: 4, SEQ ID NO: 8, SEQ ID NO: 12, or SEQ ID NO: 16. In some embodiments, are expression vectors comprising a nucleic acid sequence that encodes the amino acid sequences of SEQ ID NO: 4, SEQ ID NO: 8, SEQ ID NO: 12, or SEQ ID NO: 16. In some embodiments, are host cells comprising an expression vector comprising a nucleic acid sequence that encodes the amino acid sequences of SEQ ID NO: 4, SEQ ID NO: 8, SEQ ID NO: 12, or SEQ ID NO: 16. In some embodiments, are peptides comprising a sequence that encodes the rhodopsin domain of an anion-conducting channelrhodopsin. In some embodiments, are isolated peptides comprising an amino acid sequence of SEQ ID NO: 4, SEQ ID NO: 8, SEQ ID NO: 12, or SEQ ID NO: 16. In some embodiments, the isolated peptides comprise an amino acid sequence of SEQ ID NO: 4, SEQ ID NO: 8, SEQ ID NO: 12, or SEQ ID NO: 16, or fragments thereof.

In some embodiments, are isolated nucleic acid molecules wherein said nucleic acid molecule has a sequence of SEQ ID NO: 1-3, SEQ ID NO: 5-7, SEQ ID NO: 9-11, or SEQ ID NO: 13-15. In other embodiments, are expression vectors comprising a nucleic acid sequence selected from that shown in SEQ ID NO: 1-3, SEQ ID NO: 5-7, SEQ ID NO: 9-11, or SEQ ID NO: 13-15 and those that encode the amino acid sequences shown in SEQ ID NO: 4, SEQ ID NO: 8, SEQ ID NO: 12, or SEQ ID NO: 16. In some embodiments, are host cells comprising a expression vector comprising a nucleic acid sequence shown in SEQ ID NO: 1-3, SEQ ID NO: 5-7, SEQ ID NO: 9-11, or SEQ ID NO: 13-15 and those that encode the amino acid sequences of SEQ ID NO: 4, SEQ ID NO: 8, SEQ ID NO: 12, or SEQ ID NO: 16. In some embodiments, an isolated nucleic acid comprises a nucleotide sequence that encodes the rhodopsin domain of an anion-conducting channelrhodopsin. In some embodiments, the nucleotide sequence encodes at least 16, 20, 33, 35, or 75 contiguous amino acids of SEQ ID NO: 4, SEQ ID NO: 8, SEQ ID NO: 12, or SEQ ID NO: 16. In some embodiments, the nucleotide sequence encodes a peptide comprising any contiguous portion of SEQ ID NO: 4, SEQ ID NO: 8, SEQ ID NO: 12, or SEQ ID NO: 16.

In some embodiments, an isolated nucleic acid comprising a nucleotide sequence that encodes a functional domain of an anion-conducting channelrhodopsin. In some embodiments are isolated nucleic acid that encodes at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 175, 200, 205, 210, 215, 220, 225, 228, 229, 230, 235, 240 250, 255, 260, 265, 270, 275, 280, 285, 290, 295, 296 or more contiguous amino acids of SEQ ID NO: 4, SEQ ID NO: 8, SEQ ID NO: 12, or SEQ ID NO: 16. Further, in some embodiments, any range derivable between any of the above-described integers.

In other embodiments, there is provided an isolated polypeptide or an isolated nucleic acid encoding a polypeptide having in some embodiments between about 70% and about 75%; in further embodiments between about 75% and about 80%; in further still embodiments between about 80% and 90%; or even more further between about 90% and about 99% of amino acids (for example 95%) that are identical to (or homologous to) the amino acids of SEQ ID NO: 4, SEQ ID NO: 8, SEQ ID NO: 12, or SEQ ID NO: 16 or fragments thereof.

In other embodiments, the present invention provides for an isolated nucleic acid encoding a polypeptide having between about 70% and about 75%; or more preferably between about 75% and about 80%; or more preferably between about 80% and 90%; or even more preferably between about 90% and about 99% of amino acids that are identical to the amino acids of SEQ ID NO: 4, SEQ ID NO: 8, SEQ ID NO: 12, or SEQ ID NO: 16 or fragments thereof.

In some embodiments, the nucleic acid segments, regardless of the length of the coding sequence itself, may be combined with other DNA sequences, such as promoters, enhancers, polyadenylation signals, additional restriction enzyme sites, multiple cloning sites, other coding segments, and the like. In some embodiments, for example, are recombinant nucleic acids comprising a nucleotide sequence that encodes amino acids of SEQ ID NO: 4, SEQ ID NO: 8, SEQ ID NO: 12, or SEQ ID NO: 16 or fragments thereof, operably linked to a heterologous promoter.

In certain embodiments the invention provides an isolated nucleic acid obtained by amplification from a template nucleic acid using a primer selected from appropriate primer that can be used with SEQ ID NO: 1-3, SEQ ID NO: 5-7, SEQ ID NO: 9-11, or SEQ ID NO: 13-15.

In some embodiments, are any of the disclosed methods wherein the expression vectors include, but are not limited to, AAV viral vector. In some embodiments, are any of the disclosed methods wherein the promoter is a constitutive promoter. In some embodiments, are any of the disclosed methods wherein the constitutive promoter includes, but is not limited to, a CMV promoter or a hybrid CMV enhancer/chicken β-actin (CAG) promoter. In some embodiments, are any of the disclosed methods wherein the promoter includes, but is not limited to, an inducible and/or a cell type-specific promoter.

In some embodiments, there is provided a cDNA-derived nucleic acid comprising a nucleic acid sequence that encodes an amino acid sequence selected from SEQ ID NO: 4, SEQ ID NO: 8, SEQ ID NO: 12, or SEQ ID NO: 16 wherein the cDNA-derived nucleic acid comprises the nucleic acid sequence of SEQ ID NO: 1-3, SEQ ID NO: 5-7, SEQ ID NO: 9-11, or SEQ ID NO: 13-15. In other embodiments thee is an expression vector comprising the cDNA-derived nucleic acid comprising a nucleic acid sequence that encodes an amino acid sequence selected from SEQ ID NO: 4, SEQ ID NO: 8, SEQ ID NO: 12, or SEQ ID NO: 16.

Rhodopsin domain nucleic acid sequences for use in the disclosed methods and compositions include, but are not limited to, the active portion of the rhodopsin domains presently disclosed and encoded by the nucleic acid sequences of SEQ ID NO: 1-3, SEQ ID NO: 5-7, SEQ ID NO: 9-11, or SEQ ID NO: 13-15.

In some embodiments, the use of an active portion of a presently disclosed anion-conducting channelrhodopsin, such as but not limited to the rhodopsin domain, includes all or portions of the sequences described herein (and expression vectors comprising the same), and additionally contemplates the use of any nucleotide sequence encoding a contiguous active portion of the presently disclosed anion-conducting channelrhodopsins, such as but not limited to the rhodopsin domain, open reading frame (ORF) that hybridizes to a complement of an anion-conducting channelrhodopsin or channelopsin sequence described herein under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1 and 2 (Ausubel et al., eds., Green Publishing Associates, Incorporated, and John Wiley & Sons, Incorporated, New York, N.Y., 1989)), and encodes a functionally equivalent anion-conducting channelrhodopsin (or active portion thereof, such as but not limited to the rhodopsin domain) gene product or the active portion thereof. Additionally contemplated is the use of any nucleotide sequence that hybridizes to the complement of a DNA sequence that encodes an anion-conducting channelrhodopsin amino acid sequence under moderately stringent conditions, e.g., washing in 0.2×SSC/0.1% SDS at 42° C. (“Current Protocols in Molecular Biology”, supra), yet still encodes a functionally equivalent anion-conducting channelrhodopsin product. Functional equivalents of anion-conducting channelrhodopsin include, but are not limited to, naturally occurring versions of anion-conducting channelrhodopsin present in other or the same species (orthologs, paralogs and more generally homologs), and mutant versions of anion-conducting channelrhodopsin, whether naturally occurring or engineered (by site directed mutagenesis, gene shuffling, or directed evolution, as described in, for example, U.S. Pat. No. 5,837,458) or active portion thereof, such as but not limited to the rhodopsin domain. The disclosure also includes the use of degenerate nucleic acid variants (due to the redundancy of the genetic code) of the identified channelrhodopsin polynucleotide sequences.

Additionally contemplated is the use of polynucleotides encoding anion-conducting channelrhodopsin ORFs, or their functional equivalents, encoded by polynucleotide sequences that are about 99, 95, 90, or about 85 percent similar to the corresponding regions of the anion-conducting channelrhodopsin sequences described herein (as measured by BLAST sequence comparison analysis using, for example, the University of Wisconsin GCG sequence analysis package (SEQUENCHER 3.0, Gene Codes Corporation, Ann Arbor, Mich.) using default parameters).

The nucleic acid segments of the embodiments, regardless of the length of the coding sequence itself, may be combined with other sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, nucleic acid fragments may be prepared which include a short stretch complementary to nucleic acids that encode the polypeptides of SEQ ID NO: 4, SEQ ID NO: 8, SEQ ID NO: 12, or SEQ ID NO: 16, such as about 10 to 15 or 20, 30, or 40 or so nucleotides, and which are up to 2000 or so base pairs in length. DNA segments with total lengths of about 8000, 7000, 6000, 5000, 4000, 3000, 2000, 1000, 500, 200, 100 and about 50 base pairs in length are also contemplated to be useful.

In some embodiments, isolated nucleic acids that encode the amino acids of a channelrhodopsin or fragment thereof and recombinant vectors incorporating nucleic acid sequences which encode a channelrhodopsin protein or peptide and that includes within its amino acid sequence an amino acid sequence in accordance with SEQ ID NO: 4, SEQ ID NO: 8, SEQ ID NO: 12, or SEQ ID NO: 16. In some embodiments, a purified nucleic acid segment that encodes a protein that encodes a channelrhodopsin or fragment thereof, the recombinant vector may be further defined as an expression vector comprising a promoter operatively linked to said channelrhodopsin-encoding nucleic acid segment.

In additional embodiments, is a host cell, made recombinant with a recombinant vector comprising channelrhodopsin-encoding nucleic acid segments. The recombinant host cell may be a prokaryotic cell or a eukaryotic cell. As used herein, the term “engineered” or “recombinant” cell is intended to refer to a cell into which a recombinant gene, such as a gene encoding a channelrhodopsin, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a copy of a genomic gene or a cDNA gene, or will include genes positioned adjacent to a promoter not naturally associated with the particular introduced gene. In some embodiments, nucleic acid molecules having sequence regions consisting of contiguous nucleotide stretches of about 14, 15-20, 30, 40, 50, or even of about 100 to about 200 nucleotides or so, identical or complementary to the channelrhodopsin-encoding nucleic acid sequences.

In still further aspects, there is provided a host cell comprising a nucleic acid molecule of the embodiments (e.g., that encodes an ACR). Thus, in some embodiments a host cell is an: isolated human cell; a non-human mammalian cell; a bacterial cell; a yeast cell; an insect cell; or a plant cell. In alternative embodiments, the nucleic acid sequences described can be targeted to the genome of a cell using a CRISPR-associated protein-9 nuclease (Cas9) based system for genome-editing and genome targeting. In some embodiments, delivery to some cells may require delivery systems, such as, but not limited to those based on lentivirus (LVs), adenovirus (AdV) and adenoassociated (AAV).

An exemplary DNA binding protein is an RNA guided DNA binding protein of a Type II CRISPR System. An exemplary DNA binding protein is a Cas9 protein. According to one aspect, an engineered Cas9-gRNA system is provided which enables RNA-guided genome cutting in a site specific manner, if desired, and modification of the genome by insertion of exogenous channelrhodopsin-encoding nucleic acids provided herein. The guide RNAs are complementary to target sites or target loci on the DNA. The guide RNAs can be crRNA-tracrRNA chimeras. The Cas9 binds at or near target genomic DNA. The one or more guide RNAs bind at or near target genomic DNA. The Cas9 cuts the target genomic DNA and exogenous donor DNA is inserted into the DNA at the cut site.

Accordingly, methods are directed to the use of a guide RNA with a Cas9 protein and an exogenous channelrhodopsin-encoding nucleic acid to multiplex insertions of exogenous channelrhodopsin-encoding nucleic acids into DNA within a cell expressing Cas9 by cycling the insertion of nucleic acid encoding the RNA and exogenous donor nucleic acid, expressing the RNA, colocalizing the RNA, Cas9 and DNA in a manner to cut the DNA, and insertion of the exogenous donor nucleic acid. The method steps can be cycled in any desired number to result in any desired number of DNA modifications.

D. Recombinant Expression

While the desired peptide amino acid sequences described can be chemically synthesized (see, e.g., “Proteins: Structures and Molecular Principles” (Creighton, ed., W. H. Freeman & Company, New York, N.Y., 1984)), large polypeptides sequences may advantageously be produced by recombinant DNA technology using techniques well-known in the art for expressing nucleic acids containing a nucleic acid sequence that encodes the desired peptide. Such methods can be used to construct expression vectors containing peptide encoding nucleotide sequences and appropriate transcriptional and translational control signals. These methods include, for example, in vitro recombinant DNA techniques, synthetic techniques, and in vivo genetic recombination (see, e.g., “Molecular Cloning, A Laboratory Manual”, supra, and “Current Protocols in Molecular Biology”, supra). Alternatively, RNA and/or DNA encoding desired peptide encoding nucleotide sequences may be chemically synthesized using, for example, synthesizers (see, e.g., “Oligonucleotide Synthesis: A Practical Approach” (Gait, ed., IRL Press, Oxford, United Kingdom, 1984)).

A variety of host-expression vector systems may be utilized to express peptide encoding nucleotide sequences. When the desired peptide or polypeptide is soluble or a soluble derivative, the peptide or polypeptide can be recovered from the host cell culture, i.e., from the host cell in cases where the peptide or polypeptide is not secreted, and from the culture media in cases where the peptide or polypeptide is secreted by the host cell. However, suitable expression systems also encompass engineered host cells that express the desired polypeptide or functional equivalents anchored in the cell membrane. Purification or enrichment of the desired peptide from such expression systems can be accomplished using appropriate detergents and lipid micelles, and methods well-known to those skilled in the art. Furthermore, such engineered host cells themselves may be used in situations where it is desired not only to retain the structural and functional characteristics of the peptide, but to assess biological activity, e.g., in certain drug screening assays.

In certain applications, transient expression systems are desired. However, for long-term, high-yield production of recombinant proteins or peptides, stable expression is generally preferred. For example, cell lines that stably express the desired protein, polypeptide, peptide, or fusion protein may be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells are allowed to grow for about 1-2 days in an enriched media, and then switched to a selective media. The selectable marker in the recombinant plasmid confers resistance to the selection, and allows cells to stably integrate the plasmid into their chromosomes and grow to form foci, which in turn can be cloned and expanded into cell lines. This method may advantageously be used to engineer cell lines that express the desired gene products or portions thereof. Such engineered cell lines may be particularly useful in screening and evaluation of compounds that affect the endogenous activity of a desired protein, polypeptide or peptide.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprr cells, respectively. Anti-metabolite resistance can also be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to the aminoglycoside G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Host cells/expression systems that may be used for purpose of providing compositions to be used in the disclosed methods include, but are not limited to, microorganisms such as bacteria (e.g., E. coli, B. subtilis) transformed with a recombinant bacteriophage DNA, plasmid DNA, or cosmid DNA expression vector containing a desired peptide encoding nucleotide sequence; yeast (e.g., Saccharomyces cerevisiae, Pichia pastoris) transformed with a recombinant yeast expression vector containing a desired peptide encoding nucleotide sequence; insect cell systems infected with a recombinant virus expression vector (e.g., baculovirus) containing a desired peptide encoding nucleotide sequence; plant cell systems infected with a recombinant virus expression vector (e.g., cauliflower mosaic virus, CaMV; tobacco mosaic virus, TMV), or transformed with a recombinant plasmid expression vector (e.g., Ti plasmid), containing a desired peptide encoding nucleotide sequence; or mammalian cell systems (e.g., COS, CHO, BHK, 293, 3T3) harboring a recombinant expression construct containing a desired peptide encoding nucleotide sequence and a promoter derived from the genome of mammalian cells (e.g., metallothionein promoter) or from mammalian viruses (e.g., the adenovirus late promoter, the vaccinia virus 7.5K promoter).

In bacterial systems, a number of different expression vectors may be advantageously selected depending upon the use intended for the desired gene product being expressed. For example, when a large quantity of such a protein is to be produced, such as for the generation of pharmaceutical compositions comprising a desired peptide, or for raising antibodies to the protein, vectors that direct the expression of high levels of fusion protein products that are readily purified may be desirable. Such vectors include, but are not limited to: the E. coli expression vector pUR278 (Ruther and Müller-Hill, EMBO J. 2:1791-1794, 1983), in which a desired peptide encoding sequence may be ligated individually into the vector in frame with the lacZ coding region so that a fusion protein is produced; pIN vectors (Inouye and Inouye, Nucleic Acids Res. 13:3101-3110, 1985, and Van Heeke and Schuster, J. Biol. Chem. 264:5503-5509, 1989); and the like. pGEX vectors (GE Healthcare, Piscataway, N.J.) may also be used to express a desired peptide moiety as a fusion protein with glutathione S-transferase (GST). In general, such fusion proteins are soluble and can easily be purified from lysed cells by adsorption to glutathione-agarose beads, followed by elution in the presence of free glutathione. The pGEX vectors are designed to include thrombin or factor Xa protease cleavage sites so that the cloned desired peptide encoding gene product can be released from the GST moiety.

In an exemplary insect system, Autographa californica nuclear polyhedrosis virus (AcNPV) is used as a vector to express a desired peptide encoding sequence. The virus grows in Spodoptera frugiperda cells. A desired peptide encoding sequence may be cloned individually into a non-essential region (for example the polyhedrin gene) of the virus, and placed under control of an AcNPV promoter (for example the polyhedrin promoter). Successful insertion of a desired peptide encoding sequence will result in inactivation of the polyhedrin gene and production of non-occluded recombinant virus (i.e., virus lacking the proteinaceous coat coded for by the polyhedrin gene). The recombinant viruses are then used to infect Spodoptera frugiperda cells in which the inserted polynucleotide is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, a desired peptide encoding nucleotide sequence may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing desired peptide products in infected hosts (see, e.g., Logan and Shenk, Proc. Natl. Acad. Sci. USA 81:3655-3659, 1984). Specific initiation signals may also be required for efficient translation of inserted desired peptide encoding nucleotide sequences. These signals include the ATG initiation codon and adjacent sequences. In some cases exogenous translational control signals, including, perhaps, the ATG initiation codon, may be provided. Furthermore, the initiation codon should be in phase with the reading frame of the desired peptide encoding coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Nevins, CRC Crit. Rev. Biochem. 19:307-322, 1986).

In yeast, a number of vectors containing constitutive or inducible promoters may be used. For a review, see, e.g., “Current Protocols in Molecular Biology”, supra, Ch. 13, Bitter et al., Meth. Enzymol. 153:516-544, 1987, “DNA Cloning”, Vol. II, Ch. 3 (Glover, ed., IRL Press, Washington, D.C., 1986); Bitter, Meth. Enzymol. 152:673-684, 1987, “The Molecular Biology of the Yeast Saccharomyces: Life Cycle and Inheritance” (Strathern et al., eds., Cold Spring Harbor Press, Cold Spring Harbor, N.Y., 1981), and “The Molecular Biology of the Yeast Saccharomyces: Metabolism and Gene Expression” (Strathern et al., eds., Cold Spring Harbor Press, Cold Spring Harbor, N.Y., 1982).

In plants, a variety of different plant expression vectors can be used, and expression of a desired peptide encoding sequence may be driven by any of a number of promoters. For example, viral promoters such as the 35S RNA or 19S RNA promoters of CaMV (Brisson et al., Nature 310:511-514, 1984), or the coat protein promoter of TMV (Takamatsu et al., EMBO J. 6:307-311, 1987) may be used. Alternatively, plant promoters such as the promoter of the small subunit of RUBISCO (Coruzzi et al., EMBO J. 3:1671-1679, 1984, and Broglie et al., Science 224:838-843, 1984), or heat shock promoters, e.g., soybean hsp17.5-E or hsp17.3-B (Gurley et al., Mol. Cell. Biol. 6:559-565, 1986) may be used. These constructs can be introduced into plant cells using, for example, Ti plasmids, Ri plasmids, plant virus vectors, direct DNA transformation, microinjection, or electroporation. For reviews of such techniques, see, e.g., Weissbach and Weissbach, in “Methods in Plant Molecular Biology”, Section VIII (Schuler and Zielinski, eds., Academic Press, Inc., New York, N.Y., 1988), and “Plant Molecular Biology”, 2^{nd} Ed., Ch. 7-9 (Grierson and Covey, eds., Blackie & Son, Ltd., Glasgow, Scotland, United Kingdom, 1988).

In addition, a host cell strain may be chosen that modulates the expression of the inserted desired peptide encoding sequence, or modifies and processes the desired peptide encoding nucleic acid sequence in a desired fashion. Such modifications (e.g., glycosylation) and processing (e.g., cleavage) of protein products may affect certain functions of the protein. Different host cells have characteristic and specific mechanisms for post-translational processing and modification of proteins and peptides. Appropriate cell lines or host systems can be chosen to ensure the correct or desired modification and processing of the desired protein, polypeptide, or peptide expressed. To this end, eukaryotic host cells that possess the cellular machinery for desired processing of the primary transcript, and glycosylation and/or phosphorylation of desired peptide encoding nucleic acid sequence be used. Such mammalian host cells include, but are not limited to, Chinese hamster ovary (CHO), VERO, baby hamster kidney (BHK), HeLa, monkey kidney (COS), MDCK, 293, 3T3, WI38, human hepatocellular carcinoma (e.g., Hep G2), and U937 cells.

In some embodiments, a recombinant host cell comprising one of the nucleic acid sequences described. In some embodiments, a protein composition comprising one of the polypeptides described.

### III. METHODS OF USE

In some embodiments, molecular engineered variants (some with improved activity) of the described anion-conducting channelrhodopsin by site-specific mutagenesis and chimera construction. In some embodiments, the channelrhodopsins serve as receptors for phototaxis and the photophobic response. Their photoexcitation initiates depolarization or hyperpolarization of the cell membrane or in the case of neurons with high cytoplasmic chloride concentration, wherein the expressed rhodopsin depolarizes the plasma membrane.

In some embodiments, the rhodopsin domains of several anion-conducting channelrhodopsins were cloned and determined to have channel activity when they were expressed in mammalian HEK293 cells. Using these methods new anion-conducting channelrhodopsin variants, were determined to have improved properties with regards to, among other applications, optogenetics.

One of the major challenges for optogenetic applications, especially in living animals, are scattering of the stimulating light by biological tissues and its absorption by hemoglobin. Optogenetic tools with long-wavelength absorption would exhibit minimal light attenuation from these effects, but most microbial rhodopsins do not fall into this category. For instance, the absorption maximum of ChR2, which possesses several other useful properties and is thereby most frequently used as a depolarizing tool in optogenetics, is 470 nm.

Long-wavelength absorption by optogenetic tools is generally considered desirable to increase the penetration depth of the stimulus light by minimizing tissue scattering and absorption by hemoglobin. In some embodiments, the long-wavelength sensitivity of optogenetic microbial rhodopsins is enhanced using 3,4-Dehydroretinal (A2 retinal). A2 retinal (3,4-dehydroretinal) is a natural retinoid, its 11-cis form being found in photoreceptor cells of certain invertebrates, fish and amphibians, where it may constitute the only retinal, or an additional chromophore to A1 retinal. The presence of an additional double bond in the I3-ionone ring of the chromophore results in pigments that absorb light at longer wavelengths, as compared to those formed with A1 (regular) retinal. Variations in A1/A2 ratio cause natural adaptive tuning of spectral sensitivity of vision in the organisms during adaptation to external conditions. Reconstitution of bleached microbial rhodopsins (bacteriorhodopsin, halorhodopsin, sensory rhodopsins I and II) in vitro with all-trans 3,4-dehydroretinal (A2 retinal) also shifts their absorption spectra to longer wavelengths. In some embodiments, spectral properties of optogenetic tools were modified by incorporation of all-trans A2 retinal. The addition of A2 retinal, both ion pumps and channelrhodopsins form functional pigments with significantly red-shifted absorption.

In some embodiments, the long-wavelength sensitivity of optogenetic microbial rhodopsins is enhanced using A2 retinal. In some embodiments, chromophore substitution provides a complementary strategy to improve the efficiency of optogenetic tools. Substitution of A1 retinal by A2 retinal significantly shifts the spectral sensitivity of tested rhodopsins to longer wavelengths typically without altering other aspects of their function.

Optogenetic techniques involve the introduction of light-activated channels and enzymes that allow manipulation of neural activity and control of neuronal function. Thus, in some embodiments, the disclosed methods and compositions can be introduced into cells and facilitate the manipulation of the cells activity and function. See, for example, US publication 20130090454 of U.S. application Ser. No. 13/622,809, as well as, Mattis, J., Tye, K. M., Ferenczi, E. A., Ramakrishnan, C., O'Shea, D. J., Prakash, R., Gunaydin, L. A., Hyun, M., Fenno, L. E., Gradinaru, V., Yizhar, O., and Deisseroth, K. (2012) Principles for applying optogenetic tools derived from direct comparative analysis of microbial opsins. Nat. Methods 9, 159-172; and Zhang, F., Vierock, J., Yizhar, O., Fenno, L. E., Tsunoda, S., Kianianmo-meni, A., Prigge, M., Berndt, A., Cushman, J., Polle, J., Magnuson, J., Hege-mann, P., and Deisseroth, K. (2011) The microbial opsin family of optogenetic tools. Cell 147, 1446-1457).

Optogenetic techniques, and thus the disclosed methods and compositions, can be used to characterize the functions of complex neural circuits and information processing in the normal brain and during various neurological conditions; functionally map the cerebral cortex; characterize and manipulate the process of learning and memory; characterize and manipulate the process of synaptic transmission and plasticity; provide light-controlled induction of gene expression; provide optical control of cell motility and other activities.

Clinical applications of the disclosed methods and compositions include (but are not limited to) optogenetic approaches to therapy such as: restoration of vision by introduction of channelrhodopsins in post-receptor neurons in the retina for ocular disorder gene-therapy treatment of age-dependent macular degeneration, diabetic retinopathy, and retinitis pigmentosa, as well as other conditions which result in loss of photoreceptor cells; control of cardiac function by using channelrhodopsins incorporated into excitable cardiac muscle cells in the atrioventricular bundle (bundle of His) to control heart beat rhythm rather than an electrical pacemaker device; restoration of dopamine-related movement dysfunction in Parkinsonian patients; amelioration of depression; recovery of breathing after spinal cord injury; provide noninvasive control of stem cell differentiation and assess specific contributions of transplanted cells to tissue and network function. Any group of electrically active cells may be amenable to ACR suppression, including, but not limited to those listed above and cardiomyocytes. Such ACRs are also potentially useful for efficient photoinhibition of cardiomyocyte action potentials thereby enabling treatment of cardiac dysfunctions including, but not limited to, tachycardia. In some embodiments, the presently described compositions and methods can be used to facilitate optical stimulation of cardiac cells and tissues, without negative electrophysiological effects of current cardiac anti-arrhythmia therapies and alleviate symptoms by stimulating or silencing specific regions with abnormal excitation in the heart or the brain. In some embodiments, such optogenetic-based techniques could be used to silence, restore or reset irregular heartbeat in patients some of which now receive implantable devices. In some embodiments, ACR based reversible silencing of neuronal activity provides an effective method to isolate specific neuronal populations, thus facilitating modifications in nociceptive pain, behavior, learning and locomotion, for example, when motor neurons are silenced.

In some embodiments, the presently described compositions and methods can be used to influence cardiac cells or regions by either direct viral gene delivery (such as but not limited to AAV) or by delivery of ACR-carrying donor cells (such as but not limited to cardiomyocytes, Purkinje and His bundle cells, etc.) generated or transformed in culture.

In some embodiments, a method of membrane hyperpolarization of a cell in a subject suffering from a neuron mediated disorder, or in the case of neurons with high cytoplasmic chloride concentration, wherein the expressed rhodopsin depolarizes the plasma membrane, said method comprising: delivering to the cell of said subject an expression vector comprising a polynucleotide that encodes an amino acid sequence selected from the group consisting of SEQ ID NO: 4, SEQ ID NO: 8, SEQ ID NO: 12, or SEQ ID NO: 16, which encodes a rhodopsin domain of an anion-conducting channelrhodopsin expressible in said cell; and expressing said vector in said cell, wherein the expression of the rhodopsin results in membrane hyperpolarization or, for example, in the case of neurons with high cytoplasmic chloride concentration, wherein the expressed rhodopsin depolarizes the plasma membrane.

In some embodiments, a method of neuronal silencing in a subject suffering from a neuron mediated disorder, said method comprising: delivering to a target neuron of said subject an expression vector comprising a polynucleotide that encodes an amino acid sequence selected from the group consisting of SEQ ID NO: 4, SEQ ID NO: 8, SEQ ID NO: 12, or SEQ ID NO: 16, which encodes a rhodopsin domain of an anion-conducting channelrhodopsin expressible in said target neuron; and expressing said vector in said target neuron, wherein the expression of the rhodopsin results in silencing of the signal from the target neuron.

In some embodiments, a method of restoring photosensitivity to a retina of a subject suffering from vision loss or blindness, said method comprising: delivering to the retina of said subject an expression vector comprising a polynucleotide that encodes an amino acid sequence selected from the group consisting of SEQ ID NO: 4, SEQ ID NO: 8, SEQ ID NO: 12, or SEQ ID NO: 16 which encodes a humanized rhodopsin domain of an anion-conducting channelrhodopsin expressible in a retinal neuron; and expressing said vector in said retinal neuron, wherein the expressed rhodopsin renders a high level of membrane potential in said retinal neuron.

Therefore, in some embodiments an anion-conducting channelrhodopsin, light-gated anion channels that provide membrane hyperpolarization is provided and may be used to enhance optogenetic techniques and optogenetic approaches to therapy.

Anion-conducting channelrhodopsins, functional or active portions thereof, such as but not limited to the rhodopsin domain, and functional equivalents include, but are not limited to, naturally occurring versions of ACR and those that are orthologs and homologs, and mutant versions of ACR, whether naturally occurring or engineered (by site directed mutagenesis, gene shuffling, or directed evolution, as described in, for example, U.S. Pat. No. 5,837,458). Also included are the use of degenerate nucleic acid variants (due to the redundancy of the genetic code) of the disclosed algae ACR derived polynucleotide sequences.

In some embodiments, are methods of treating a neuronal disorder, comprising: (a) delivering to a target neuron a nucleic acid expression vector that encodes a rhodopsin domain of an anion-conducting channelrhodopsin, expressible in said target neuron, said vector comprising an open reading frame encoding the rhodopsin domain of an anion-conducting channelrhodopsin, operatively linked to a promoter sequence, and optionally, a transcriptional regulatory sequence; and (b) expressing said vector in said target neuron, wherein the expressed rhodopsin that results in membrane hyperpolarization and neuronal silencing of said target neuron upon exposure to light. In some embodiments, the rhodopsin domain is encoded by SEQ ID NO: 1-3, SEQ ID NO: 5-7, SEQ ID NO: 9-11, or SEQ ID NO: 13-15.

In some embodiments, are methods of treating a neuronal disorder, comprising: (a) delivering to a target neuron a nucleic acid expression vector that encodes a rhodopsin domain of an anion-conducting channelrhodopsin derived from algae, expressible in said target neuron, said vector comprising an open reading frame encoding the rhodopsin domain of an anion-conducting channelrhodopsin, operatively linked to a promoter sequence, and optionally, a transcriptional regulatory sequence; and (b) expressing said vector in said target neuron, wherein the expressed rhodopsin silences said target neuron upon exposure to light. In some embodiments, the rhodopsin domain is encoded by SEQ ID NO: 1-3, SEQ ID NO: 5-7, SEQ ID NO: 9-11, or SEQ ID NO: 13-15.

In some embodiments, are methods of restoring light sensitivity to a retina, comprising: (a) delivering to a retinal neuron a nucleic acid expression vector that encodes a rhodopsin domain of an anion-conducting channelrhodopsin, expressible in the retinal neuron; said vector comprising an open reading frame encoding the rhodopsin domain of an anion-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 1-3, SEQ ID NO: 5-7, SEQ ID NO: 9-11, or SEQ ID NO: 13-15.

In some embodiments, are methods of restoring light sensitivity to a retina, comprising: (a) delivering to a retinal neuron a nucleic acid expression vector that encodes a rhodopsin domain of an anion-conducting channelrhodopsin, expressible in the retinal neuron; said vector comprising an open reading frame encoding the rhodopsin domain of an anion-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 1-3, SEQ ID NO: 5-7, SEQ ID NO: 9-11, or SEQ ID NO: 13-15.

In some embodiments, are methods of restoring photosensitivity to a retina of a subject suffering from vision loss or blindness in whom retinal photoreceptor cells are degenerating or have degenerated and died, said method comprising: (a) delivering to the retina of said subject a nucleic acid vector that encodes a rhodopsin domain of an anion-conducting channelrhodopsin expressible in a retinal neuron; said vector comprising an open reading frame encoding the rhodopsin domain of an anion-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring photosensitivity to said retina or a portion thereof. In some embodiments, the rhodopsin domain is encoded by SEQ ID NO: 1-3, SEQ ID NO: 5-7, SEQ ID NO: 9-11, or SEQ ID NO: 13-15.

In some embodiments, the presently described compositions and methods can be used to facilitate optical stimulation of cardiac cells and tissues, without negative electrophysiological effects of current cardiac anti-arrhythmia therapies and alleviate symptoms by stimulating or silencing specific regions with abnormal excitation in the heart or the brain. In some embodiments, such optogenetic based techniques could be used to silence, restore or reset irregular heartbeat in patients some of which now receive implantable devices.

In some embodiments, the presently described compositions and methods can be used to influence cardiac cells or regions by either direct viral gene delivery (such as but not limited to AAV) or by delivery of ACR-carrying donor cells (such as but not limited to cardiomyocytes, Purkinje and His bundle cells, etc.) generated or transformed in culture.

In some embodiments is a method of treating a disorder in an electrically active cell in a subject suffering from a disorder that involves electrically active cells, said method comprising: (a) delivering to the cell of said subject an expression vector comprising a polynucleotide that encodes an amino acid sequence selected from SEQ ID NO: 4, SEQ ID NO: 8, SEQ ID NO: 12, or SEQ ID NO: 16 expressible in said cell; and (b) expressing said vector in said electrically active cell, wherein the expressed rhodopsin silences the signal from said electrically active cell.

In some embodiments is a method of treating a disorder in an electrically active cell in a subject suffering from a disorder that involves electrically active cells, said method comprising: (a) delivering to said subject a transgenic cell comprising an expression vector comprising a polynucleotide that encodes an amino acid sequence selected from SEQ ID NO: 4, SEQ ID NO: 8, SEQ ID NO: 12, or SEQ ID NO: 16 expressible in said transgenic cell; and (b) expressing said vector in said transgenic cell, wherein the expression silences the signal from a electrically active cell.

In some embodiments is a method of silencing an electrically active cell in a subject suffering from an electrically active cell mediated disorder, said method comprising: (a) delivering to a target neuron of said subject an expression vector comprising a polynucleotide that encodes an amino acid sequence selected from SEQ ID NO: 4, SEQ ID NO: 8, SEQ ID NO: 12, or SEQ ID NO: 16 which encodes a rhodopsin domain of an anion-conducting channelrhodopsin expressible in said target neuron; and (b) expressing said vector in said target electrically active cell, wherein the expressed rhodopsin results in silencing of the signal from the electrically active cell. In some embodiments there is provided a recombinant host cell, wherein said host cell is an isolated electrically active cell.

In some embodiments, a method of treating a neuronal disorder comprises (a) delivering to a target neuron a nucleic acid expression vector that encodes a rhodopsin domain of an anion-conducting channelrhodopsin, expressible in said target neuron; said vector comprising an open reading frame encoding the rhodopsin domain of an anion-conducting channelrhodopsin operatively linked to a promoter sequence, and optionally, transcriptional regulatory sequences; and (b) expressing the expression vector in the target neuron, wherein the expressed anion-conducting channelrhodopsin silences the target neuron upon exposure to light. In some embodiments an above-described expression vector also comprises one or more transcriptional regulatory sequences operably linked to the promoter and rhodopsin domain sequences. In some embodiments, the rhodopsin domain of a anion-conducting channelrhodopsin has the amino acid sequence of all or part of SEQ ID NO: 4, SEQ ID NO: 8, SEQ ID NO: 12, or SEQ ID NO: 16 and the rhodopsin domain sequences of SEQ ID NO: 4, SEQ ID NO: 8, SEQ ID NO: 12, or SEQ ID NO: 16, or a biologically active fragment thereof that retains the biological activity of the encoded rhodopsin domain of a channelrhodopsin or is a biologically active conservative amino acid substitution variant of SEQ ID NO: 4, SEQ ID NO: 8, SEQ ID NO: 12, or SEQ ID NO: 16 or of said fragment. In some embodiments, the expression vector comprises an AAV viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring light sensitivity to a retina comprises (a) delivering to a retinal neuron in a subject a nucleic acid expression vector that encodes a rhodopsin domain of an anion-conducting channelrhodopsin, expressible in the retinal neuron; said expression vector comprising an open reading frame encoding the rhodopsin domain of an anion-conducting channelrhodopsin operatively linked to a promoter sequence, and optionally, one or more transcriptional regulatory sequences; and (b) expressing the expression vector in the retinal neuron, wherein the expressed rhodopsin renders the retinal neuron photosensitive, thereby restoring light sensitivity to the retina or a portion thereof. In some embodiments, the rhodopsin domain of the anion-conducting channelrhodopsin has the amino acid sequence of all or part of SEQ ID NO: 4, SEQ ID NO: 8, SEQ ID NO: 12, or SEQ ID NO: 16, or a biologically active fragment thereof that retains the biological activity of the encoded rhodopsin domain of an anion-conducting channelrhodopsin or is a biologically active conservative amino acid substitution variant of SEQ ID NO: 4, SEQ ID NO: 8, SEQ ID NO: 12, or SEQ ID NO: 16, or of said fragment. In some embodiments, the expression vector comprises an AAV (e.g., AAV2) viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring photosensitivity to a retina of a subject suffering from vision loss or blindness in whom retinal photoreceptor cells are degenerating or have degenerated and died comprises: (a) delivering to the retina of the subject a nucleic acid expression vector that encodes a rhodopsin domain of an anion-conducting channelrhodopsin expressible in retinal neurons; said expression vector comprising an open reading frame encoding the rhodopsin domain of an anion-conducting channelrhodopsin operatively linked to a promoter sequence, and optionally, transcriptional regulatory sequences; and (b) expressing the expression vector in the retinal neuron, wherein the expression of the rhodopsin renders the retinal neuron photosensitive, thereby restoring photosensitivity to said retina or a portion thereof. In some embodiments, the rhodopsin domain of an anion-conducting channelrhodopsin has the amino acid sequence of all or part of SEQ ID NO: 4, SEQ ID NO: 8, SEQ ID NO: 12, or SEQ ID NO: 16 or the rhodopsin domain sequences of SEQ ID NO: 4, SEQ ID NO: 8, SEQ ID NO: 12, or SEQ ID NO: 16, or a biologically active fragment thereof that retains the biological activity of the encoded rhodopsin domain of an anion-conducting channelrhodopsin or is a biologically active conservative amino acid substitution variant SEQ ID NO: 4, SEQ ID NO: 8, SEQ ID NO: 12, or SEQ ID NO: 16, or of said fragment. In some embodiments, the expression vector comprises an AAV (e.g., AAV2) viral vector. In some embodiments, the promoter is a constitutive promoter. In some embodiments, the constitutive promoter is a CMV promoter or a hybrid CMV enhancer/chicken β-actin (CAG) promoter. In other embodiments, the promoter is an inducible and/or a cell type-specific promoter.

B. Compositions as Therapeutics

The use of channelrhodopsins, or active fragments thereof such as but not limited to the rhodopsin domain as therapeutics. In certain embodiments the presently disclosed compositions and are used to improve optogenetic techniques and applications as well as can be used to aid in diagnosis, prevention, and/or treatment of among other things neuron mediated disSorders, neurologic disorders (such as Parkinson's disease) and as therapy for ocular disorders.

In certain embodiments the presently disclosed compositions can be administered in combination with one or more additional compounds or agents (“additional active agents”) for the treatment, management, and/or prevention of among other things neuron mediated disorders, neurologic disorders (such as Parkinson's disease) and as therapy for ocular disorders. Such therapies can be administered to a patient at therapeutically effective doses to treat or ameliorate, among other things, neuron mediated disorders, neurologic disorders (such as Parkinson's disease) and as therapy for ocular disorders. A therapeutically effective dose refers to that amount of the compound sufficient to result in any delay in onset, amelioration, or retardation of disease symptoms.

Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, expressed as the ratio LD₅₀/ED₅₀. Compositions that exhibit large therapeutic indices are preferred. Compounds that exhibit toxic side effects may be used in certain embodiments, however, care should usually be taken to design delivery systems that target such compositions preferentially to the site of affected tissue, in order to minimize potential damage to uninfected cells and, thereby, reduce side effects.

Data obtained from cell culture assays and animal studies can be used in formulating a range of dosages for use in humans. The dosages of such compositions lie preferably within a range of circulating concentrations that include the ED₅₀ with little or no toxicity. The dosage may vary within this range depending on the dosage form employed and the route of administration utilized. For any composition, the therapeutically effective dose can be estimated initially from cell culture assays. A dose may be formulated in animal models to achieve a circulating plasma concentration range that includes the IC₅₀ (i.e., the concentration of the test composition that achieves a half-maximal inhibition of symptoms) as determined in cell culture. Such information can be used to more accurately determine useful doses in humans. Plasma levels may be measured, for example, by high performance liquid chromatography.

When the therapeutic treatment of among other things neurologic disorders (such as Parkinson's disease) and as therapy for ocular disorders is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies help establish safe doses.

Additionally, the bioactive agent may be coupled or complexed with a variety of well-established compositions or structures that, for instance, enhance the stability of the bioactive agent, or otherwise enhance its pharmacological properties (e.g., increase in vivo half-life, reduce toxicity, etc.).

Such therapeutic agents can be administered by any number of methods known to those of ordinary skill in the art including, but not limited to, inhalation, subcutaneous (sub-q), intravenous (I.V.), intraperitoneal (I.P.), intramuscular (I.M.), or intrathecal injection, or topically applied (transderm, ointments, creams, salves, eye drops, and the like), as described in greater detail below.

C. Pharmaceutical Compositions

Pharmaceutical compositions for use in accordance with the presently described compositions may be formulated in conventional manners using one or more physiologically acceptable carriers or excipients.

The pharmaceutical compositions can comprise formulation materials for modifying, maintaining, or preserving, for example, the pH, osmolarity, viscosity, clarity, color, isotonicity, odor, sterility, stability, rate of dissolution or release, adsorption or penetration of the composition. Suitable formulation materials include, but are not limited to: amino acids (for example, glycine, glutamine, asparagine, arginine and lysine); antimicrobials; antioxidants (for example, ascorbic acid, sodium sulfite and sodium hydrogen-sulfite); buffers (for example, borate, bicarbonate, Tris-HCl, citrates, phosphates and other organic acids); bulking agents (for example, mannitol and glycine); chelating agents (for example, ethylenediamine tetraacetic acid (EDTA)); complexing agents (for example, caffeine, polyvinylpyrrolidone, beta-cyclodextrin, and hydroxypropyl-beta-cyclodextrin); fillers; monosaccharides, disaccharides, and other carbohydrates (for example, glucose, mannose and dextrins); proteins (for example, serum albumin, gelatin and immunoglobulins); coloring, flavoring, and diluting agents; emulsifying agents; hydrophilic polymers (for example, polyvinylpyrrolidone); low molecular weight polypeptides; salt-forming counterions (for example, sodium); preservatives (for example, benzalkonium chloride, benzoic acid, salicylic acid, thimerosal, phenethyl alcohol, methylparaben, propylparaben, chlorhexidine, sorbic acid and hydrogen peroxide); solvents (for example, glycerin, propylene glycol and polyethylene glycol); sugar alcohols (for example, mannitol and sorbitol); suspending agents; surfactants or wetting agents (for example, pluronics, PEG, sorbitan esters, polysorbates (for example, polysorbate 20 and polysorbate 80), triton, tromethamine, lecithin, cholesterol, and tyloxapal); stability enhancing agents (for example, sucrose and sorbitol); tonicity enhancing agents (for example, alkali metal halides (for example, sodium or potassium chloride), mannitol, and sorbitol); delivery vehicles; diluents; excipients; and pharmaceutical adjuvants (“Remington's Pharmaceutical Sciences”, 18^{th} Ed. (Gennaro, ed., Mack Publishing Company, Easton, Pa., 1990)).

Additionally, the described therapeutic peptides can be linked to a half-life extending vehicle. Certain exemplary half-life extending vehicles are known in the art, and include, but are not limited to, the Fc domain, polyethylene glycol, and dextran (see, e.g., PCT Patent Application Publication No. WO 99/25044).

These agents may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The agents may also be formulated as compositions for rectal administration such as suppositories or retention enemas, e.g., containing conventional suppository bases such as cocoa butter or other glycerides.

In addition to the formulations described previously, the agents may also be formulated as a depot preparation. Such long acting formulations may be administered by implantation (for example subcutaneously or intramuscularly) or by intramuscular injection. For example, agents may be formulated with suitable polymeric or hydrophobic materials (for example as an emulsion in an acceptable oil), ion exchange resins, or as sparingly soluble derivatives, for example, as a sparingly soluble salt. The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

Active compositions can be administered by controlled release means or by delivery devices that are well-known to those of ordinary skill in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, and 5,733,566. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Exemplary sustained release matrices include, but are not limited to, polyesters, hydrogels, polylactides (see, e.g., U.S. Pat. No. 3,773,919 and European Patent Application Publication No. EP 058,481), copolymers of L-glutamic acid and gamma ethyl-L-glutamate (see, e.g., Sidman et al., Biopolymers 22:547-556, 1983), poly (2-hydroxyethyl-methacrylate) (see, e.g., Langer et al., J. Biomed. Mater. Res. 15:167-277, 1981, and Langer, Chemtech 12:98-105, 1982), ethylene vinyl acetate (Langer et al., supra), and poly-D(−)-3-hydroxybutyric acid (European Patent Application Publication No. EP 133,988). Sustained release compositions may include liposomes, which can be prepared by any of several methods known in the art (see, e.g., Eppstein et al., Proc. Natl. Acad. Sci. USA 82:3688-3692, 1985, and European Patent Application Publication Nos. EP 036,676, EP 088,046, and EP 143,949). Suitable controlled-release formulations known to those of ordinary skill in the art, including those described herein, can be readily selected for use with the presently disclosed compositions. Certain embodiments encompass single unit dosage forms suitable for oral administration such as, but not limited to, tablets, capsules, gelcaps, and caplets that are adapted for controlled-release.

All controlled-release pharmaceutical products have a common goal of improving therapy over that achieved by their non-controlled counterparts. Ideally, use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of active ingredient that promptly produces the desired therapeutic effect, and gradually and continually release other amounts of active ingredient to maintain this level of therapeutic or prophylactic effect over an extended period of time. In order to maintain this relatively constant level of active ingredient in the body, the drug must be released from the dosage form at a rate that will replace the amount of active ingredient being metabolized and excreted from the body. Controlled-release of an active ingredient can be stimulated by various conditions including, but not limited to, pH, temperature, enzymes, water, or other physiological conditions or compositions.

In some cases, active ingredients of the disclosed methods and compositions are preferably not administered to a patient at the same time or by the same route of administration. Therefore, in some embodiments are kits that, when used by the medical practitioner, can simplify the administration of appropriate amounts of active ingredients to a patient.

A typical kit comprises a single unit dosage form of one or more of the therapeutic agents disclosed, alone or in combination with a single unit dosage form of another agent that may be used in combination with the disclosed compositions. Disclosed kits can further comprise devices that are used to administer the active ingredients. Examples of such devices include, but are not limited to, syringes, drip bags, patches, and inhalers.

Disclosed kits can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. However, in specific embodiments, the disclosed formulations do not contain any alcohols or other co-solvents, oils or proteins.

D. Transgenic Animals

The present disclosure provides methods and compositions for the creation and use of both human and non-human transgenic animals that carry an algae derived anion-conducting channelrhodopsin transgene in all their cells, as well as non-human transgenic animals that carry an algae derived anion-conducting channelrhodopsin transgene in some, but not all their cells, for example in certain electrically active cells. Human and non-human mammals of any species, including, but not limited to, mice, rats, rabbits, guinea pigs, pigs, micro-pigs, goats, and non-human primates, e.g., baboons, monkeys, and chimpanzees, can be used to generate transgenic animals carrying an algae derived anion-conducting channelrhodopsin polynucleotide (and/or expressing an algae derived polypeptide) may be integrated as a single transgene or in concatamers, e.g., head-to-head or head-to-tail tandems. An algae derived anion-conducting channelrhodopsin transgene may also be selectively introduced into and activated in a particular cell-type (see, e.g., Lakso et al., Proc. Natl. Acad. Sci. USA 89:6232-6236, 1992). The regulatory sequences required for such a cell-type specific activation will depend upon the particular cell-type of interest, and will be apparent to those of skill in the art.

Should it be desired that an algae-derived anion-conducting channelrhodopsin, or fragment thereof, transgene be integrated into the chromosomal site of the endogenous copy of the mammalian anion-conducting channelrhodopsin gene, gene targeting is generally preferred. Briefly, when such a technique is to be utilized, vectors containing some nucleotide sequences homologous to the endogenous anion-conducting channelrhodopsin gene are designed for the purpose of integrating, via homologous recombination with chromosomal sequences, into and disrupting the function of the endogenous channelrhodopsin gene (i.e., “knock-out” animals). In this way, the expression of the endogenous channelrhodopsin gene may also be eliminated by inserting non-functional sequences into the endogenous channelrhodopsin gene. The transgene may also be selectively introduced into a particular cell-type, thus inactivating the endogenous channelrhodopsin gene in only that cell-type (see, e.g., Gu et al., Science 265:103-106, 1994). The regulatory sequences required for such a cell-type specific inactivation will depend upon the particular cell-type of interest, and will be apparent to those of skill in the art.

Any technique known in the art may be used to introduce a channelrhodopsin, or fragment thereof, transgene into animals to produce the founder lines of transgenic animals. Such techniques include, but are not limited to: pronuclear microinjection (U.S. Pat. No. 4,873,191); retrovirus-mediated gene transfer into germ lines (van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); sperm-mediated gene transfer (Lavitrano et al., Cell 57:717-723, 1989); and positive-negative selection, as described in U.S. Pat. No. 5,464,764. For a review of such techniques, see, e.g., Gordon, Int. Rev. Cytol. 115:171-229, 1989.

Once transgenic animals have been generated, the expression of the recombinant channelrhodopsin gene, or fragment thereof, may be assayed utilizing standard techniques. Initial screening may be accomplished by Southern blot analysis or PCR techniques to analyze animal tissues to assay whether integration of the channelrhodopsin transgene has taken place. The level of mRNA expression of the channelrhodopsin transgene in the tissues of the transgenic animals may also be assessed using techniques that include, but are not limited to, Northern blot analysis of cell-type samples obtained from the animal, in situ hybridization analysis, and RT-PCR. Samples of an algae derived channelrhodopsin-expressing tissue can also be evaluated immunocytochemically using antibodies selective for the channelrhodopsin transgene product.

E. Transgene Based Therapies

In certain embodiments the presently disclosed compositions and are used to improve optogenetic techniques and applications as well as can be used to aid in diagnosis, prevention, and/or treatment of neurologic disorders, such as but not limited to Parkinson's disease, as well as for ocular disorders.

In some embodiments, methods and compositions are used to identify and characterize multiple channelrhodopsins derived from algae. The cloning and expression of the rhodopsin domain of the channelrhodopsins and expression in mammalian cells demonstrates that these channelrhodopsins have improved characteristics that can be used for optogenetic applications as well as therapeutic agents.

For example, a disclosed method and composition may be used in, among other things, retinal gene therapy for mammals (as described in, among others, U.S. Pat. Nos. 5,827,702, 7,824,869 and US Patent Publication Number 20100015095 as well as in WIPO publications WO 2000/15822 and WO 1998/48097). A genetically engineered ocular cell is produced by contacting the cell with an exogenous nucleic acid under conditions in which the exogenous nucleic acid is taken up by the cell for expression. The exogenous nucleic acid is described as a retrovirus, an adenovirus, an adeno-associated virus or a plasmid. Retinal gene transfer of a reporter gene, green fluorescent protein (GFP), using a recombinant adeno-associated virus (rAAV) was demonstrated in normal primates (Bennett, J et al. 1999 Proc. Natl. Acad. Sci. USA 96, 9920-25). The rescue of photoreceptors using gene therapy in a model of rapid degeneration of photoreceptors using mutations of the RP65 gene and replacement therapy with the normal gene to replace or supplant the mutant gene (See, for example, US Patent Publication 2004/0022766) has been used to treat a naturally-occurring dog model of severe disease of retinal degenerations—the RPE65 mutant dog, which is analogous to human LCA. By expressing photosensitive membrane-channels or molecules in surviving retinal neurons of the diseased retina by viral based gene therapy method, the present invention may produce permanent treatment of the vision loss or blindness with high spatial and temporal resolution for the restored vision.

The nucleic acids sequences that encode an active portion of the presently disclosed anion-conducting channelrhodopsins, include but are not limited to the nucleic acid sequences that encode the rhodopsin domains identified in SEQ ID NO: 4, SEQ ID NO: 8, SEQ ID NO: 12, or SEQ ID NO: 16 or the optimized rhodopsin domain-encoding sequences of SEQ ID NO: 1-3, SEQ ID NO: 5-7, SEQ ID NO: 9-11, or SEQ ID NO: 13-15.

In some embodiments, introduction and expression of channelrhodopsins, such as those described herein, in ocular neuronal cells, for example, impart light sensitivity to such retinas and restoring one or more aspects of visual responses and functional vision to a subject suffering from such degeneration. By restoring light sensitivity to a retina lacking this capacity, due to disease, a mechanism for the most basic light-responses that are required for vision is provided. In some embodiments, the functional domains of anion-conducting channelrhodopsins, such as GtACR1 and GtACR2 may be used to restore light sensitivity to the retinas that have undergone rod and cone degeneration by expressing the channelrhodopsin in inner retinal neurons in vivo. In some embodiments these channelrhodopsins may be introduced using techniques that include, but are not limited to, retinal implants, cortical implants, lateral geniculate nucleus implants, or optic nerve implants

In some embodiments, the anion-conducting channelrhodopsins are inserted into the retinal neurons that survived after the rods and cones have died in an area or portion of the retina of a subject, using the transfer of nucleic acids, alone or within an expression vector. Such expression vectors may be constructed, for example, by introduction of the desired nucleic acid sequence into a virus system known to be of use for gene therapy applications, such as, but not limited to, AAV (e.g., AAV2), retroviruses and alike.

In some embodiments the anion-conducting channelrhodopsins may be inserted into retinal interneurons. These cells then can become light sensitive and send signals via the optic nerve and higher order visual pathways to the visual cortex where visual perception occurs, as has been demonstrated electrophysiologicly in mice. In some embodiments, among other routes, intravitreal and/or subretinal injections may be used to deliver channelrhodopsin molecules or virus vectors expressing the same.

In some embodiments, the active portion of the presently disclosed algal derived anion-conducting channelrhodopsins, such as but not limited to the rhodopsin domain of these anion-conducting channelrhodopsins, can be used to restore light sensitivity to a retina, by delivering to retinal neurons a nucleic acid expression vector that encodes algal derived anion-conducting channelrhodopsins (such as but not limited to the rhodopsin domain of these anion-conducting channelrhodopsins) that is expressible in the neurons, which vector comprises an open reading frame encoding the rhodopsin, and operatively linked thereto, a promoter sequence, and optionally, transcriptional regulatory sequences; and expressing the vector in the neurons, thereby restoring light sensitivity.

In certain embodiments the channel rhodopsin can be algal derived anion-conducting channelrhodopsins such as, but not limited to functional domains of anion-conducting channelrhodopsins, such as, but not limited to, SEQ ID NO: 4, SEQ ID NO: 8, SEQ ID NO: 12, or SEQ ID NO: 16 or a biologically active fragment or conservative amino acid substitution variant thereof, such as but not limited to the rhodopsin domain. The vector system may be recombinant AAV (e.g., AAV2), the promoter may be a constitutive promoter such as, but not limited to, a CMV promoter or a hybrid CMV enhancer/chicken β-actin promoter (CAG).

### IV. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, and unless otherwise indicated, the term a disorder that involves electrically active cells, such as but not limited to neuronal dysfunction, a neuron mediated disorder, ocular disorder or cardiac disorder, for which the present methods and compositions may be used include, but are not limited to, neuronal dysfunctions, disorders of the brain, the central nervous system, the peripheral nervous system, neurological conditions, disorders of memory and leaning disorders, cardiac arrhythmias, Parkinson's disease, epilepsy, ocular disorders, spinal cord injury, nerve pain associated with, but not limited to autoimmune diseases (for example, multiple sclerosis, Guillain-Barré syndrome, myasthenia gravis, lupus, and inflammatory bowel disease); cancer and the chemotherapy and radiation used to treat it; compression/trauma (for example, pinched nerves in the neck, crush injuries, and carpal tunnel syndrome); diabetic neuropathy; medication side effects; and toxic substances; motor neuron diseases (for example amyotrophic lateral sclerosis, progressive bulbar palsy, progressive muscular atrophy and primary lateral sclerosis); nutritional deficiencies (for example vitamins B6 and B12); Infectious disease; itch sensations associated with, but not limited to eczema, atopic dermatitis, dry skin and allergic itches; diseases and disorders that alter vagal nerve activity, among others.

As used herein, and unless otherwise indicated, the term ocular disorders for which the present methods and compositions may be used to improve one or more parameters of vision include, but are not limited to, developmental abnormalities that affect both anterior and posterior segments of the eye. Anterior segment disorders include, but are not limited to, glaucoma, cataracts, corneal dystrophy, and keratoconus. Posterior segment disorders include, but are not limited to, blinding disorders caused by photoreceptor malfunction and/or death caused by retinal dystrophies and degenerations. Retinal disorders include congenital stationary night blindness, age-related macular degeneration, congenital cone dystrophies, and a large group of retinitis pigmentosa (RP)—related disorders.

As used herein, and unless otherwise indicated, the terms “treat,” “treating,” “treatment” and “therapy” contemplate an action that occurs while a patient is suffering from a disorder that involves electrically active cells, such as but not limited to neuronal dysfunction, a neuron mediated disorder, ocular disorder or cardiac disorder, and which reduces the severity of one or more symptoms or effect of such a disorder. Where the context allows, the terms “treat,” “treating,” and “treatment” also refers to actions taken toward ensuring that individuals at increased risk of a disorder that involves electrically active cells, such as but not limited to neuronal dysfunction, a neuron mediated disorder, ocular disorder or cardiac disorder and which reduces the severity are able to receive appropriate surgical and/or other medical intervention prior to onset of a disorder that involves electrically active cells, such as but not limited to neuronal dysfunction, a neuron mediated disorder, ocular disorder or cardiac disorder and which reduces the severity. As used herein, and unless otherwise indicated, the terms “prevent,” “preventing,” and “prevention” contemplate an action that occurs before a patient begins to suffer from a disorder that involves electrically active cells, such as but not limited to neuronal dysfunction, a neuron mediated disorder, ocular disorder or cardiac disorder, that delays the onset of, and/or inhibits or reduces the severity of a disorder that involves electrically active cells, such as but not limited to neuronal dysfunction, a neuron mediated disorder, ocular disorder or cardiac disorder.

As used herein, and unless otherwise indicated, the terms “manage,” “managing,” and “management” encompass preventing, delaying, or reducing the severity of a recurrence of a disorder that involves electrically active cells, such as but not limited to neuronal dysfunction, a neuron mediated disorder, ocular disorder or cardiac disorder in a patient who has already suffered from such a disease, disorder or condition. The terms encompass modulating the threshold, development, and/or duration of the disorder that involves electrically active cells or changing how a patient responds to the disorder that involves electrically active cells or the maintenance and/or establishment of a desirable membrane potential across the membrane of a cell.

As used herein, and unless otherwise specified, a “therapeutically effective amount” of a compound is an amount sufficient to provide any therapeutic benefit in the treatment or management of a disorder that involves electrically active cells, such as but not limited to neuronal dysfunction, a neuron mediated disorder, ocular disorder or cardiac disorder, or to delay or minimize one or more symptoms associated with a disorder that involves electrically active cells, such as but not limited to neuronal dysfunction, a neuron mediated disorder, ocular disorder or cardiac disorder. A therapeutically effective amount of a compound means an amount of the compound, alone or in combination with one or more other therapies and/or therapeutic agents that provide any therapeutic benefit in the treatment or management of a disorder that involves electrically active cells, such as but not limited to neuronal dysfunction, a neuron mediated disorder, ocular disorder or cardiac disorder.

The term “therapeutically effective amount” can encompass an amount that alleviates a disorder that involves electrically active cells, such as but not limited to neuronal dysfunction, a neuron mediated disorder, ocular disorder or cardiac disorder, improves or reduces a disorder that involves electrically active cells or improves overall therapy, or enhances the therapeutic efficacy of another therapeutic agent.

As used herein, and unless otherwise specified, a “prophylactically effective amount” of a compound is an amount sufficient to prevent or delay the onset of a disorder that involves electrically active cells, such as but not limited to neuronal dysfunction, a neuron mediated disorder, ocular disorder or cardiac disorder or one or more symptoms associated with a disorder that involves electrically active cells or prevent or delay its recurrence. A prophylactically effective amount of a compound means an amount of the compound, alone or in combination with one or more other treatment and/or prophylactic agent that provides a prophylactic benefit in the prevention of a disorder that involves electrically active cells, such as but not limited to neuronal dysfunction, a neuron mediated disorder, ocular disorder or cardiac disorder. The term “prophylactically effective amount” can encompass an amount that prevents a disorder that involves electrically active cells, such as but not limited to neuronal dysfunction, a neuron mediated disorder, ocular disorder or cardiac disorder, improves overall prophylaxis, or enhances the prophylactic efficacy of another prophylactic agent. The “prophylactically effective amount” can be prescribed prior to, for example, the development of a disorder that involves electrically active cells, such as but not limited to neuronal dysfunction, a neuron mediated disorder, ocular disorder or cardiac disorder.

As used herein, “patient” or “subject” includes mammalian organisms which are capable of suffering from a disorder that involves electrically active cells, such as but not limited to neuronal dysfunction, a neuron mediated disorder, ocular disorder or cardiac disorder, as described herein, such as human and non-human mammals, for example, but not limited to, rodents, mice, rats, non-human primates, companion animals such as dogs and cats as well as livestock, e.g., sheep, cow, horse, etc.

As used herein, the term “conservative substitution” generally refers to amino acid replacements that preserve the structure and functional properties of a protein or polypeptide. Such functionally equivalent (conservative substitution) peptide amino acid sequences include, but are not limited to, additions or substitutions of amino acid residues within the amino acid sequences encoded by a nucleotide sequence that result in a silent change, thus producing a functionally equivalent gene product. Conservative amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

As used herein, a “redshift” is a shift to longer wavelength. In contrast a “blueshift” would be a shift to shorter wavelength. These terms apply to both light-emitting and light-absorbing objects.

As used herein the phrase “rhodopsin domain” refers to the “rhodopsin fold”, a 7-transmembrane-helix (7TM) structure characteristic of rhodopsins. As used herein, the channelopsin is the apoprotein, while channelrhodopsin is the protein and retinal. As used herein the term “channelrhodopsin” describes retinylidene proteins (rhodopsins) that function as light-gated ion channels.

The percent identity or homology is determined with regard to the length of the relevant amino acid sequence. Therefore, if a polypeptide of the present invention is comprised within a larger polypeptide, the percent homology is determined with regard only to the portion of the polypeptide that corresponds to the polypeptide of the present invention and not the percent homology of the entirety of the larger polypeptide. “Percent identity” or “% identity,” with reference to nucleic acid sequences, refers to the percentage of identical nucleotides between at least two polynucleotide sequences aligned using the Basic Local Alignment Search Tool (BLAST) engine. See Tatusova et al. (1999) FEMS Microbiol Lett. 174:247-250. The BLAST engine is provided to the public by the National Center for Biotechnology Information (NCBI), Bethesda, Md. To align two polynucleotide sequences, the BLAST which employs the “blastn” program is used.

“Percent identity” or “% identity,” with reference to polypeptide sequences, refers to the percentage of identical amino acids between at least two polypeptide sequences aligned using the Basic Local Alignment Search Tool (BLAST) engine. See Tatusova et al. (1999) ibid. The BLAST engine is provided to the public by the National Center for Biotechnology Information (NCBI), Bethesda, Md. To align two polypeptide sequences, the BLAST which employs the “blastp” program is used.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials & Methods

Bioinformatics. A keyword search of gene annotations was used to identify rhodopsin genes at the genome portals of A. limacinum MYA-1381, P. antarctica CCMP1374 and P. globosa Pg-G of the US Department of Energy JGI. The three hits showing protein sequence homology to previously known algal channelrhodopsins found in the A. limacinum genome were then used for tblastn search of the JGI genome portal for Schizochytrium aggregatum ATCC 28209 and whole-genome shotgun contigs from stramenopiles at the NCBI portal.

Protein sequence alignments were created using MUSCLE algorithm implemented in DNASTAR Lasergene (Madison, Wis.) MegAlign Pro software. Phylogenetic trees were analyzed with W-IQ-TREE online tool (Trifinopoulos et al., 2016) using automatic model selection, 1000 ultrafast bootstrap replicates and visualized with iTOL 5.5.1 (Letunic and Bork, 2019). Homology models were obtained using the commonly used structure prediction servers I-TASSER (Yang et al., 2015) and Robetta (Ovchinnikov et al., 2018; Haas et al., 2019), a membrane protein-adapted form of the Rosetta program, and visualized with PyMol software (available on the world wide web at pymol.org). A comparative study of several homology modeling protocols applied to microbial rhodopsins with known crystal structures (Nikolaev et al., 2018) found that I-TASSER and Rosetta with membrane-specific terms are overall highly accurate but with less fidelity in prediction of active site residue orientation. These homology models are first approximations and determination of the physical chemical basis of tuning responsible for the red-shift will require a more accurate atomic structure of a RubyACR, which is being pursued by X-ray crystallography. The best templates for AlACR1 modeling were 3 ug9A (hybrid channelrhodopsin C1C2), root-mean-square deviation (RMSD) 2.68 selected by the I-TASSER server, and 5zihB (Chrimson), RMSD 1.47, by the Robetta server.

Molecular biology. For expression in HEK293 cells, DNA polynucleotides encoding the transmembrane domains showing homology to previously known ACRs optimized for human codon usage were synthesized (GenScript, Piscataway, N.J.) and cloned into the mammalian expression vector pcDNA3.1 (Life Technologies, Grand Island, N.Y.) in frame with an EYFP or mCherry tag. Mutants were generated using Quikchange XL kit (Agilent Technologies, Santa Clara, Calif.) and verified by sequencing. For expression in Pichia, the opsin-encoding constructs were fused in frame with a C-terminal 8-His tag and subcloned into the pPIC9K (AlACR1, HfACR1 and AlACR2) or pPICZa (AlACR3) vector (Invitrogen) according to the manufacturer's instructions. Expression of the constructs for AlACR1 from Aurantiochytrium sp. and TlACR from Thraustochytrium sp. in HEK293 cells was poor, as judged by the tag fluorescence, but a C-terminal truncation to 270 encoded residues improved it. Constructs encoding the entire predicted polypeptides for AlACR1 and AlACR2 from Aurantiochytrium limacinum (696 and 635 residues, respectively) were also synthesized, but in both cases no membrane fluorescence was observed.

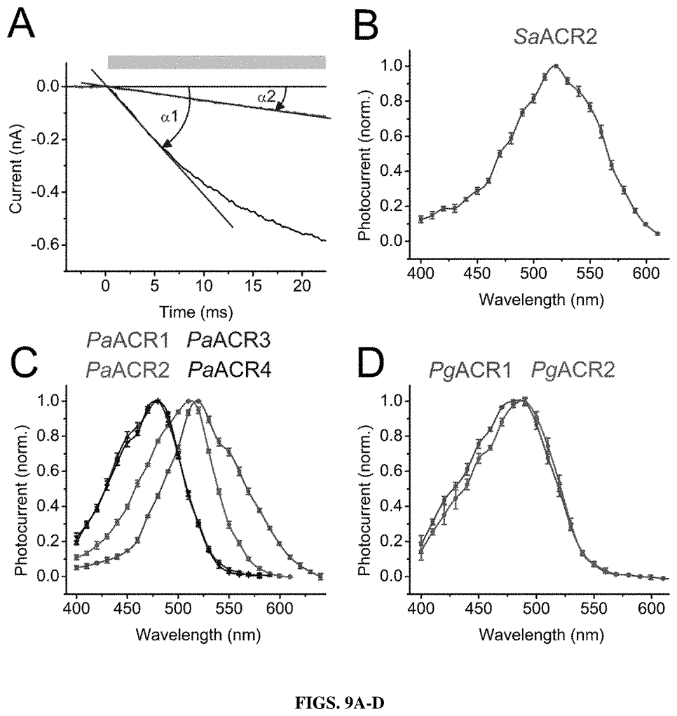

HEK293 transfection and patch clamp recording. HEK293 cells were transfected using the JetPRIME transfection reagent (Polyplus, Illkirch, France). All-trans-retinal (Sigma) was added at the final concentration of 3 μM immediately after transfection. Photocurrents were recorded 48-96 h after transfection in the whole-cell voltage clamp mode with an Axopatch 200B amplifier (Molecular Devices, Union City, Calif.) using the 10 kHz low-pass Bessel filter. The signals were digitized with a Digidata 1440A using pClamp 10 software (both from Molecular Devices). Patch pipettes with resistances of 2-4 MΩ were fabricated from borosilicate glass. The standard pipette solution contained (in mM): KCl 126, MgCl₂ 2, CaCl₂ 0.5, Na-EGTA 5, HEPES 25, pH 7.4. The standard bath solution contained (in mM): NaCl 150, CaCl₂ 1.8, MgCl₂ 1, glucose 5, HEPES 10, pH 7.4. A 4 M KCl bridge was used in all experiments, and possible diffusion of Cl⁻ from the bridge to the bath was minimized by frequent replacement of the bath solution with fresh buffer. For measurements of the reversal potential shifts under varied ionic conditions, Na⁺ was substituted for K⁺ in the pipette solution to minimize the number of ionic species in the system. To reduce the Cl⁻ concentration in the bath, NaCl was replaced with Na-aspartate; to reduce the Na⁺ concentration, with N-methyl-D-glucamine chloride; to increase the H⁺ concentration, pH was adjusted with H₂SO₄. To mimic ionic conditions of mature neurons, the pipette solution contained (in mM): K-gluconate 135, MgCl₂ 2, HEPES 20, pH 7.2, and the bath solution contained (in mM): NaCl 125, KCl 2, MgCl₂ 1, CaCl₂ 3, glucose 30, HEPES 25, pH 7.3. The holding voltages were corrected for liquid junction potentials calculated using the Clampex built-in LJP calculator (Barry, 1994). Continuous light pulses were provided by a Polychrome V light source (T.I.L.L. Photonics GMBH, Grafelfing, Germany) in combination with a mechanical shutter (Uniblitz Model LS6, Vincent Associates, Rochester, N.Y.; half-opening time 0.5 ms). The action spectra were constructed by calculation of the initial slope of photocurrent as shown in FIG. 9A and corrected for the quantum density measured at each wavelength. Laser excitation was provided by a Minilite Nd:YAG laser (532 nm, pulsewidth 6 ns, energy 12 mJ; Continuum, San Jose, Calif.). The current traces were logarithmically filtered using custom software. Curve fitting was performed by Origin Pro software (OriginLab Corporation, Northampton, Mass.).

Expression and purification of ACRs from Pichia. The plasmids encoding labyrinthulea ACRs were linearized with SalI or PmeI and used to transform P. pastoris strain SMD1168 (his4, pep4) by electroporation according to the manufacturer's instructions. Resistant transformants were screened on 4 mg/mL geneticin or 1 mg/mL zeocin and first cultivated on a small scale. Rhodopsin gene expression was induced by the addition of methanol. All-trans retinal (5 μM final concentration) was added simultaneously. Clones of the brightest color were selected for further experimentation. For protein purification, a starter culture was inoculated into buffered complex glycerol medium until A600 reached 4-8, after which the cells were harvested by centrifugation and resuspended at A600 1-2 in buffered complex methanol medium supplemented with 5 μM all-trans retinal (Sigma Aldrich). Expression was induced by the addition of 0.5% methanol. After 24-30 h, the cells were harvested and disrupted in a bead beater (BioSpec Products, Bartlesville, Okla.) in buffer A (20 mM sodium phosphate, pH 7.4, 100 mM NaCl, 1 mM EDTA, 5% glycerol). After removing cell debris by low-speed centrifugation, membrane fragments were collected by ultracentrifugation, resuspended in buffer B (20 mM Hepes, pH 7.4, 300 mM NaCl, 5% glycerol) and solubilized by incubation with 1.5% dodecyl maltoside (DDM) for 1.5 h or overnight at 4° C. Non-solubilized material was removed by ultracentrifugation, and the supernatant was mixed with nickel-nitrilotriacetic acid or cobalt superflow agarose beads (Thermofisher) and loaded on a column. The proteins were eluted with buffer C (20 mM Hepes, pH 7.4, 300 mM NaCl, 5% glycerol, 0.02% DDM) containing 300 mM imidazole, which was removed by repetitive washing with imidazole-free buffer C using YM-10 centrifugal filters (Amicon, Billerica, Mass.).

Absorption spectroscopy. Absorption spectra of purified proteins were recorded using a Cary 4000 spectrophotometer (Varian, Palo Alto, Calif.).

Statistics. The data are presented as mean±s.e.m. values. Normality and equal variances of the data were not assumed, and therefore the non-parametric two-sided Mann-Whitney test was used for pairwise comparison of independent data sets. P values >0.05 were considered not significant. The sample size was estimated from previous experience and published work on a similar subject, as recommended by the NIH guidelines (Dell et al., 2002).

### Example 2—Channelrhodopsin Sequences

Channelrhodopsin homologs were found in the genomes of twelve strains of labyrinthulea from the family Thraustochytriaceae. Most of the analyzed genomes (listed in Table 1) encode three paralogs. The exceptions are the genomes of Schizochytrium aggregatum ATCC 28209, in which only two paralogs were found, and of Aplanochytrium kerguelense PBS07, in which no homologs were found. The Aurantiochytrium sp. KH105 genome encodes three pairs of nearly identical paralogs that may have resulted from recent gene duplications in a single genome or from two different strains/species in a mixed culture. Some other sequences from different organisms were also completely or nearly identical to each other. The mean length of the encoded polypeptides was ˜660 residues. The seven transmembrane helical (rhodopsin) domain comprised ˜270 residues and was followed by a large cytoplasmic fragment, as in previously known algal channelrhodopsins. In the cytoplasmic fragments of some labyrinthulea homologs signal receiver domains (REC, accession number c119078; also known as response regulator (RR) or CheY-like domains) were detected by bioinformatic analysis, although the E-values were very large (0.01). Very recently, such domains have also been identified in the cytoplasmic fragment of green algae CCRs and ACRs from prasinophytes and their viruses (Rozenberg et al., 2020). Based on residue conservation analysis, it is suggested that, in contrast to the earlier known homologous domains associated with histidine kinase rhodopsins (Mukherjee et al., 2019), these domains in channelrhodopsins do not undergo phosphorylation and either are constitutively active or have no signaling function (Rozenberg et al., 2020).

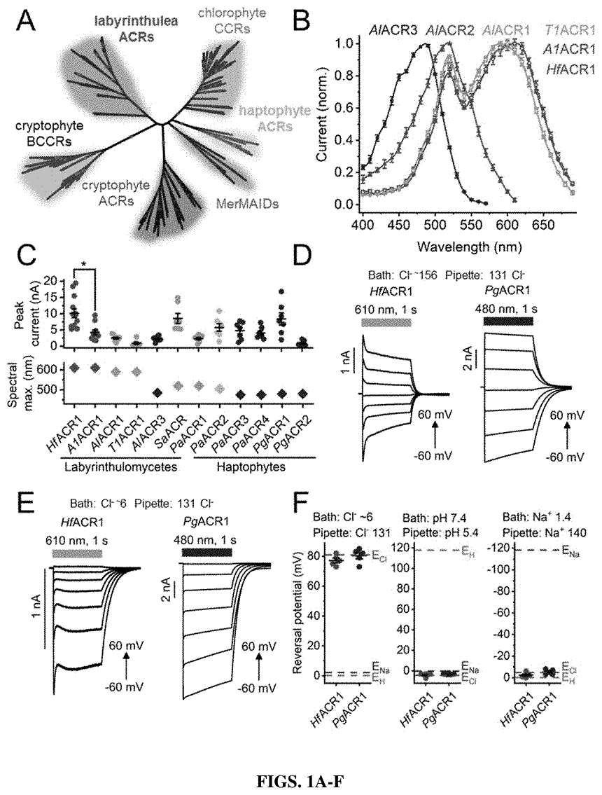

Also, six and three channelrhodopsin homologs were found in the genomes of the haptophytes Phaeocystis antarctica and P. globosa, respectively. The length of the encoded polypeptides ranges from 312 to 1682 amino acid residues. Besides the rhodopsin domain, no other known conserved domains were detected even in the longest sequence. A search of the whole-genome shotgun (WGS) contigs at the National Center for Biotechnology Information (NCBI) also returned two sequences from Stramenopiles sp. TOSAG23-3. Their rhodopsin domains, however, clustered with MerMAIDs (Metagenomically discovered, Marine, Anion-conducting and Intensely Desensitizing channelrhodopsins (Oppermann et al., 2019)), whereas labyrinthulea and haptophyte homologs formed separate branches of the phylogenetic tree of channelrhodopsins (FIG. 1A).

The GenBank accession numbers, abbreviated protein names, source organism names and gene model/WGS sequence numbers of the identified homologs are listed in Tables 2 and 3. The first two italicized letters of the protein names were derived from the genus and species name of the source organism; if no species name was assigned to a strain, consecutive numbers were used to distinguish orthologs. When numbering paralogs, a historical numbering convention was followed with the number one assigned to the most red-shifted paralog, and other paralogs consecutively numbered according to the position of their spectral maxima. The numbers were assigned to untested labyrinthulea homologs by taking into account their sequence homology with tested ones.

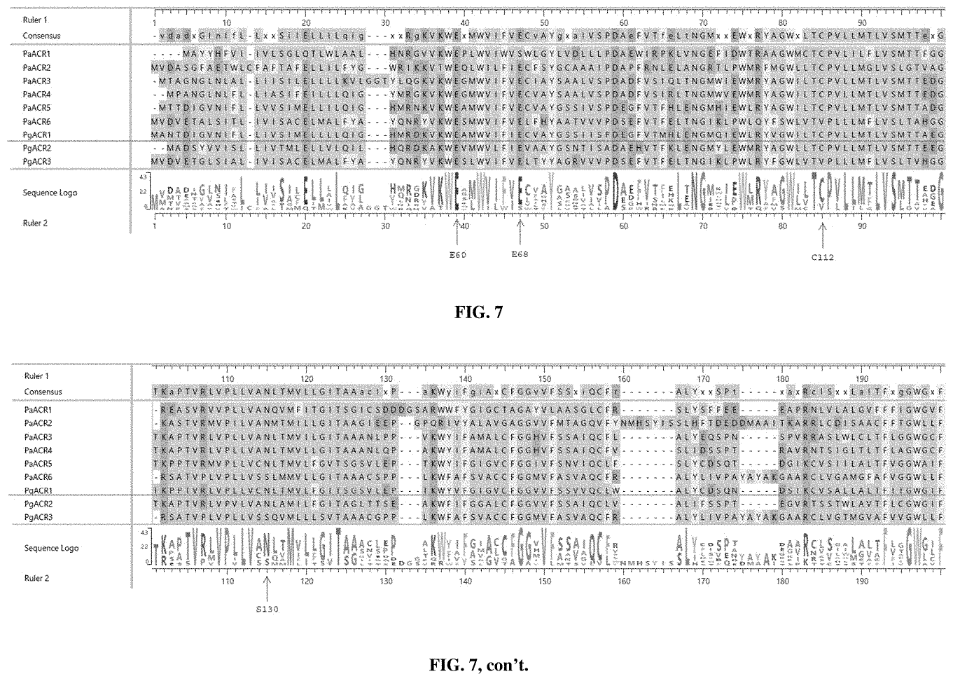

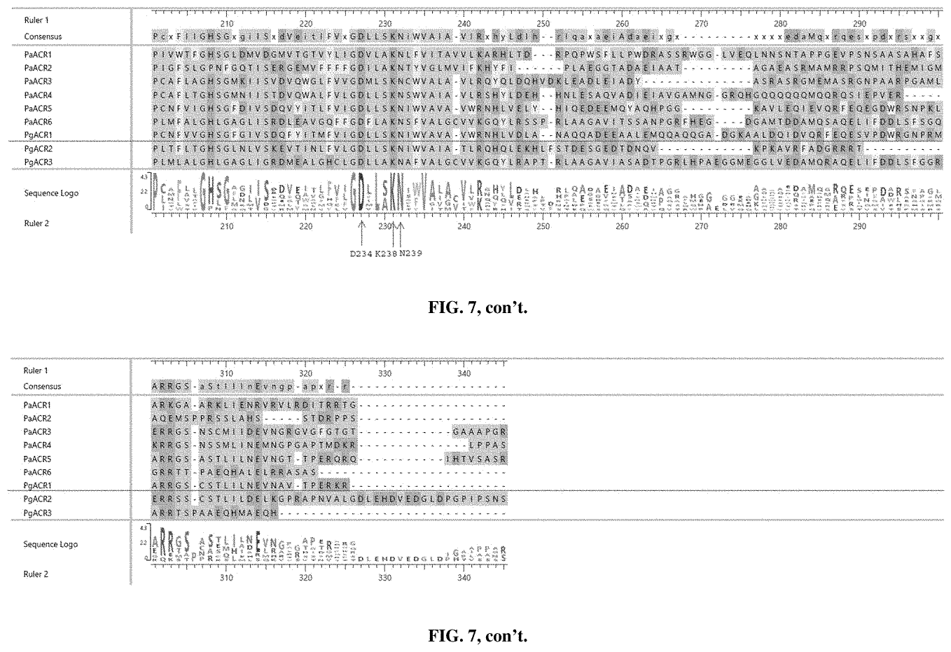

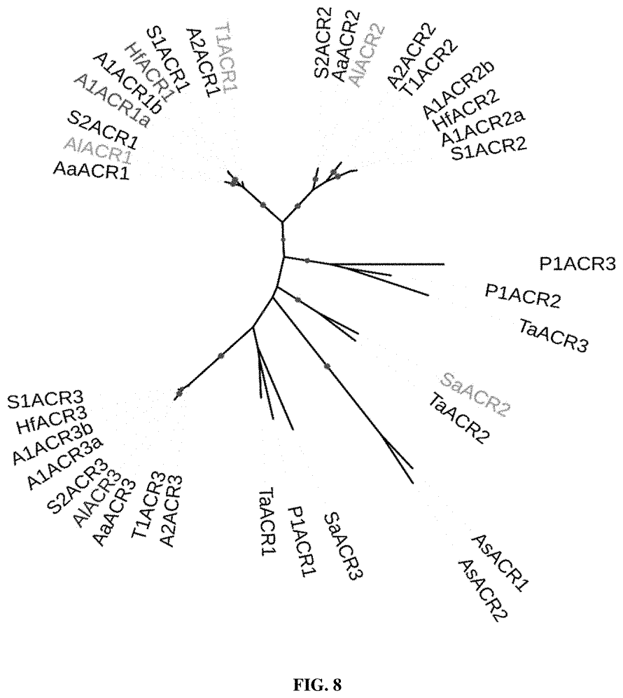

Protein alignments of the rhodopsin domains of labyrinthulea and haptophyte channelrhodopsins are shown in FIGS. 6 and 7, respectively. The Schiff base Lys is found in the seventh transmembrane helix (TM7) of all homologs, as is the immediately following Gln, characteristic of most earlier known channelrhodopsins except BCCRs. The position of the proton donor in bacteriorhodopsin (Asp85) is occupied by a non-carboxylate residue in all the sequences, as in cryptophyte and MerMAID ACRs. This feature distinguishes them from chlorophyte CCRs and cryptophyte “bacteriorhodopsin-like” CCRs (BCCRs), most of which contain a carboxylate residue in this position. The second photoactive site carboxylate (Asp212 of bacteriorhodopsin) is universally conserved. Only two glutamates (Glu60 and Glu68 using Guillardia theta ACR1 (GtACR1) numbering) are conserved in TM2 of the haptophyte homologs. This is again a feature shared with cryptophyte and MerMAID ACRs, in contrast to chlorophyte CCRs, most of which contain four conserved glutamates in this helix. Most labyrinthulea homologs completely lack glutamates in TM2, except two sequences from Aplanochytrium stocchinoi recently identified in the Marine Microbial Eukaryote Transcriptome Sequencing Project (MMETSP) database (Rozenberg et al., 2020), in which the homolog of Glu60 is conserved (FIG. 6). An unrooted phylogenetic tree of labyrinthulea homologs is shown in FIG. 8.

TABLE 1
A list of analyzed genomes

| | | Genome | | Principal | |
|---|---|---|---|---|---|
| | Organism | assembly | | investigator/ | |
| | name | number | URL | Submitter | Status |
| 1. | Aplanochytrium | 1.0 | phycocosm.jgi.doe.gov/ | Jackie | Complete, |
| | kerguelense | | Aplke1/Aplke1.home.ht | Collier | annotated |
| | PBS07 | | ml | | |
| 2. | Aurantiochytrium | ASM433257v1 | world wide web at | Heliae | Shotgun |
| | acetophilum | | ncbi.nlm.nih.gov/genom | Development | |
| | HS399 | | e/79056?genome_assem | | |
| | | | bly_id=492055 | | |
| 3. | Aurantiochytrium | Not defined | phycocosm.jgi.doe.gov/ | Jackie | Complete, |
| | limacinum | | Aurli1/Aurli1.home.html | Collier | annotated |
| | ATCC MYA- | | | | |
| | 1381 | | | | |
| 4. | Aurantiochytrium | Auran_KH105_ | world wide web at | Okinawa | Shotgun |
| | sp. KH105 | 1.0 | ncbi.nlm.nih.gov/genom | Institute of | |
| | | | e/41915?genome_assem | Science | |
| | | | bly_id=374513 | and | |
| | | | | Technology | |
| 5. | | Not defined | marinegenomics.oist.jp/ | Okinawa | Not |
| | | | aurantiochytrium_sp_kh | Institute of | defined, |
| | | | 105/viewer?project_id= | Science | annotated |
| | | | 56 | and | |
| | | | | Technology | |
| 6. | Aurantiochytrium | ASM146250v1 | world wide web at | ThraustoE | Shotgun |
| | sp. T66 | | ncbi.nlm.nih.gov/genom | ng | |
| | | | e/41915?genome assem | | |
| | | | bly_id=259575 | | |
| 7. | Hondaea | Aurantiochytriu | world wide web at | Fermental | Shotgun |
| | fermentalgiana | m_FCC1311_v | ncbi.nlm.nih.gov/genom | g | |
| | FCC1311 | 1 | e/72081?genome_assem | | |
| | (Aurantiochytrium | | bly_id=400191 | | |
| | sp. | | | | |
| | FCC1311) | | | | |
| 8. | Schizochytrium | Not defined | mycocosm.jgi.doe.gov/S | Jackie | Complete, |
| | aggregatum | | chag1/Schag1.home.html | Collier | annotated |
| | ATCC 28209 | | | | |
| 9. | Schizochytrium | ASM81894v1 | world wide web at | Nanjing | Shotgun |
| | sp. CCTCC | | ncbi.nlm.nih.gov/genom | Tech | |
| | M209059 | | e/35766?genome_assem | University | |
| | | | bly_id=217282 | | |
| 10. | Schizochytrium | ASM476469v1 | world wide web at | Third | Shotgun |
| | sp. TIO01 | | ncbi.nlm.nih.gov/genom | Institute of | |
| | | | e/35766?genome_assem | Oceano- | |
| | | | bly_id=491898 | graphy | |
| 11. | Thraustochytrium | Tau_assembly0 | world wide web at | Kyushu | Shotgun |
| | aureum | 1 | ncbi.nlm.nih.gov/genom | University | |
| | ATCC 34304 | | e/88990?genome_assem | | |
| | | | bly_id=889105 | | |
| 12. | Thraustochytrium | ASM215423v1 | world wide web at | University | Shotgun |
| | sp. ATCC | | ncbi.nlm.nih.gov/genom | of | |
| | 26185 | | e/54615?genome_assem | Saskatchewan | |
| | | | bly_id=318815 | | |
| 13. | Parietichytrium | Pari_assembly0 | world wide web at | Kyushu | Shotgun |
| | sp. I65-24A | 1 | ncbi.nlm.nih.gov/genom | University | |
| | | | e/88991?genome_assem | | |
| | | | bly_id=889106 | | |
| 14. | Phaeocystis | 2.2 | phycocosm.jgi.doe.gov/ | Kevin | Not |
| | antarctica | | Phaant1/Phaant1.home.h | Arrigo | defined, |
| | CCMP1374 | | tml | | annotated |
| 15. | Phaeocystis | 2.3 | mycocosm.jgi.doe.gov/P | Andy | Not |
| | globosa Pg-G | | haglo1/Phaglo1.home.ht | Allen | defined, |
| | | | ml | | annotated |

TABLE 2
A list of labyrinthulea ACR homologs (in bold - synthesized and tested by patch clamp
in this study)

| | GenBank | Abbreviated | | JGI gene model name | |
|---|---|---|---|---|---|
| | accession | protein | | or WGS/MMETSP | λₘₐₓ |
| | number | name | Source organism | sequence name | (nm) |
| 1. | | AaACR1 | Aurantiochytrium | QDJC01000532 | |
| 2. | | AaACR2 | acetophilum HS399 | QDJC01003161 | |
| 3. | | AaACR3 | | QDJC01000037 | |
| 4. | MT002467 | AlACR1 | Aurantiochytrium | fgenesh1_pg.12_#_284 | 590 |
| 5. | MT002473 | AlACR2 | limacinum ATCC MYA- | fgenesh1_pg.12_#_285 | 545 |
| 6. | MT002476 | AlACR3 | 1381 | fgenesh1_pg.1_#_498 | 485 |
| 7. | | AsACR1 | Aplanochytrium | DN5182_c0_g3_i1.p1 | |
| 8. | | AsACR2 | stocchinoi* | DN9014_c0_g1_i1.p1, | |
| | | | | DN12229_c0_g1_i1.pl | |
| 9. | MT002468 | A1ACRla | Aurantiochytrium sp. | BGKB01000037 | 610 |
| 10. | | A1ACR1b | KH105 | BGKB01000105 | |
| 11. | | A1ACR2a | | BGKB01000037† | |
| 12. | | A1ACR2b | | BGKB01000105† | |
| 13. | | A1ACR3a | | BGKB01000099 | |
| 14. | | A1ACR3b | | BGKB01000102 | |
| 15. | | A2ACR1 | Aurantiochytrium sp. T66 | LNGJ01004228 | |
| 16. | | A2ACR2 | | LNGJ01004228† | |
| 17. | | A2ACR3 | | LNGJ01002066 | |
| 18. | MT002469 | HfACR1 | Hondaea fermentalgiana | BEYU01000006 | 610 |
| 19. | | HfACR2 | FCC1311 | BEYU01000006† | |
| 20. | | HfACR3 | (Aurantiochytrium sp. | BEYU01000001 | |
| | | | FCC1311) | | |
| 21. | | P1ACR1 | Parietichytrium sp. I65- | BLSF01000016 | |
| 22. | | P1ACR2 | 24A | BLSF01000022 | |
| 23. | | P1ACR3 | | BLSF01000101 | |
| 24. | MT002463 | SaACR2 | Schizochytrium | fgenesh1_pg.3_#_476 | 520 |
| 25. | | SaACR3 | aggregatum ATCC 28209 | fgenesh1_pg.3_#_475 | |
| 26. | | S1ACR1 | Schizochytrium sp. CCTCC | JTFK01000019 | |
| 27. | | S1ACR2 | M209059 | JTFK01000019† | |
| 28. | | S1ACR3 | | JTFK01000324 | |
| 29. | | S2ACR1 | Schizochytrium sp. TIO01 | SMSO01000032 | |
| 30. | | S2ACR2 | | SMSO01000032† | |
| 31. | | S2ACR3 | | SMSO01000014 | |
| 32. | | TaACR1 | Thraustochytrium aureum | BLSG01000269 | |
| 33. | | TaACR2 | ATCC 34304 | BLSG01000269† | |
| 34. | | TaACR3 | | BLSG01000370 | |
| 35. | MT002470 | T1ACR1 | Thraustochytrium sp. | MUFY01006470 | 590 |
| 36. | | T1ACR2 | ATCC 26185 | MUFY01006469 | |
| 37. | | T1ACR3 | | MUFY01009420 | |

*This species was analyzed by Rozenberg et al. (16).
†These proteins are encoded by the complement strand of the WGS sequence.

TABLE 3
A list of haptophyte ACR homologs tested in this study

| | GenBank | | | | |
|---|---|---|---|---|---|
| | accession | Abbreviated | Source | JGI gene | λₘₐₓ |
| | number | protein name | organism | model name | (nm) |
| 1. | MT002471 | PaACR1 | Phaeocystis | Phant.0066s0015.1 | 520 |
| 2. | MT002474 | PaACR2 | antarctica | Phant.0011s0329.1 | 510 |
| 3. | MT002477 | PaACR3 | CCMP1374 | Phant.0016s0461.1, | 480 |
| | | | | Phant.0016s0462.1, | |
| | | | | Phant.0016s0464.1 | |
| 4. | MT002464 | PaACR4 | | Phant.0060s0074.1 | 480 |
| 5. | MT002465 | PaACR5 | | Phant.0086s0086.1 | N.A. |
| 6. | MT002466 | PaACR6 | | Phant.0001s0932.1 | N.A. |
| 7. | MT002472 | PgACR1 | Phaeocystis | Phglo.0395s0005.1 | 485 |
| 8. | MT002475 | PgACR2 | globosa Pg-G | Phglo.0149s0014.1 | 485 |
| 9. | MT002478 | PgACR3 | | Phglo.0128s0040.1 | N.A. |

### Example 3—Screening of ACR Homologs by Patch Clamp Electrophysiology

Mammalian codon-adapted polynucleotides were synthesized encoding the rhodopsin domains (residues 1-270 or 1-300, see Methods) of seven channelrhodopsin homologs from various labyrinthulea species, six from Phaeocystis antarctica and three from P. globosa, fused to a C-terminal enhanced fluorescent yellow protein (EYFP) tag, and expressed in human embryonic kidney (HEK293) cells. As shown below, labyrinthulea and haptophyte homologs are strictly anion-selective, so they are referred to as “ACRs”. The expression construct sequences were deposited to GenBank (their accession numbers are listed in the second column in Tables 2 and 3).

All seven labyrinthulea and six haptophyte homologs generated photocurrents when probed with whole-cell patch clamping. First, their action spectra were determined by measuring the initial slope in the linear range of the stimulus intensity, as shown in FIG. 9A. The spectra of AlACR1, AlACR2 and AlACR3 peaked at 590, 520 and 485 nm, respectively (FIG. 1B), reminiscent of the red, green and blue color system of human vision. AlACR1, HfACR1 and TlACR1 sequences, although they originate from different organisms, are 76-80% identical to that of AlACR1 (FIG. 6). In the action spectrum of TlACR1 photocurrents the position of the main peak was identical to that of AlACR1 (590 nm), but in both AlACR1 and HfACR1 it was at 610 nm (FIG. 1B). Other tested homologs exhibited maximal sensitivity to green or blue light (FIGS. 1C and 9B-D).

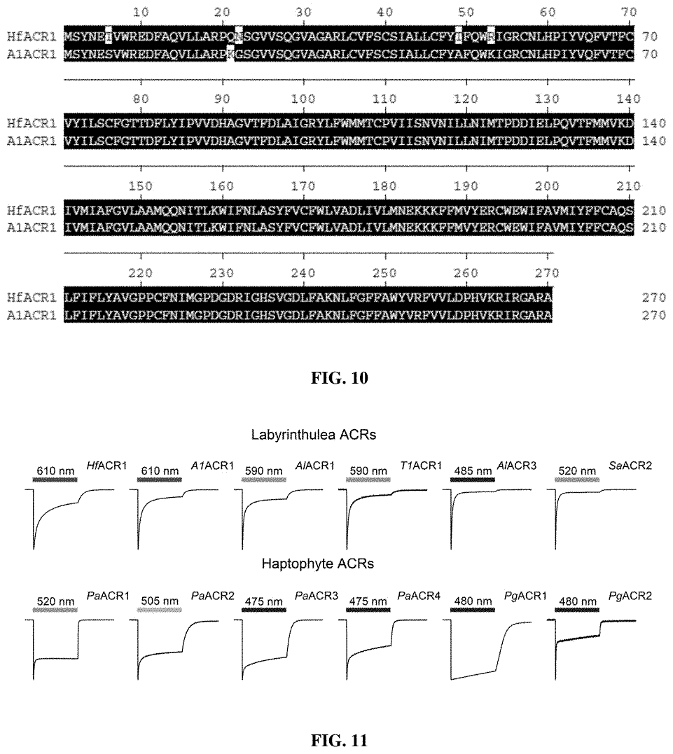

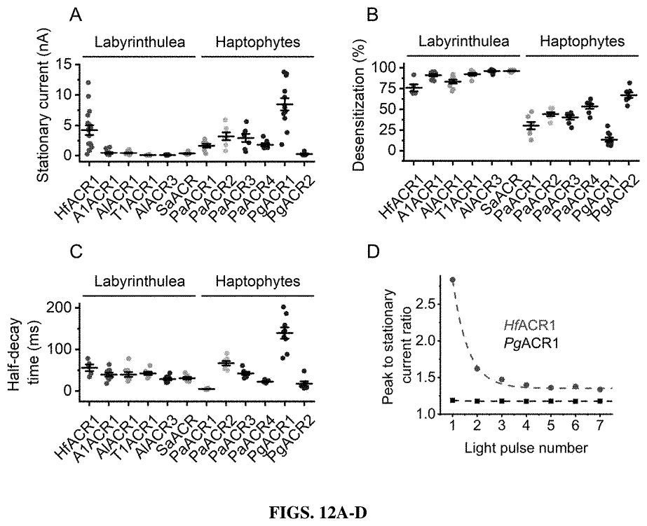

When probed at the wavelength of their maximal sensitivity, most of the tested rhodopsins generated photocurrents in the nA range. FIG. 1C shows the peak current amplitude for all tested homologs except AlACR2, the currents of which are described in a separate section below. HfACR1 generated the largest currents among labyrinthulea homologs, and PgACR1, among the haptophyte homologs. Of the two most red-shifted homologs, HfACR1 generated significantly larger photocurrents than AlACR1 (p<0.001, Mann-Whitney test), although the sequences of their rhodopsin domains differ only at five positions in the range of residues 1-270 (FIG. 10). FIG. 11 shows normalized photocurrent traces recorded from all functional homologs except AlACR2. The amplitude of channel currents decreased during continuous illumination (a phenomenon known as desensitization). The amplitude of stationary current, degree of desensitization, and half-time of current decay after switching off the light varied between different homologs (FIGS. 12A-C). On average, desensitization was greater in labyrinthulea homologs (89±3%, mean±sem, n=6 homologs) than in haptophyte homologs (41±8%, mean±sem, n=6 homologs). As in earlier studied strongly desensitizing channelrhodopsins (3), the peak-to-stationary current ratio in labyrinthulea ACRs reduced in a series of light pulses, whereas in haptophyte ACRs this effect was negligible (FIG. 12D).

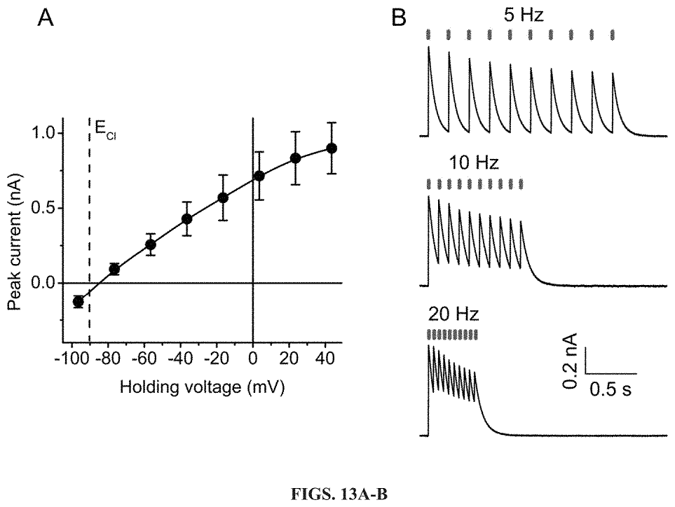

With standard bath and pipette solutions (˜156 and 131 mM Cl⁻, respectively; for other components see Methods) the photocurrents reversed their direction near zero voltage, indicating passive ionic conductance (FIG. 1D). To test relative permeability for Cl⁻, Na⁺ and H⁺, the concentrations of these ions were individually reduced in the bath. Representative photocurrent traces recorded from HfACR1 and PgACR1 with ˜6 mM Cl⁻ in the bath at incremental voltages are shown in FIG. 1E. The current-voltage relationships were then measured, and the reversal potentials (Eᵣₑᵥ) calculated. Under all tested conditions the Eᵣ, matched the Nernst equilibrium potential for Cl⁻ (FIG. 1F), indicating that the tested channelrhodopsins were strictly permeable for anions. FIG. 13A shows a current-voltage curve for HfACR1 activated at 630 nm and tested under ionic conditions typical of mature neurons (see Methods). FIG. 13B shows photocurrent traces recorded upon pulsed stimulation at different frequencies.

### Example 4—Color Tuning in Labyrinthulea ACRs

Comparison of the retinal-binding pockets of AlACR1 (spectral maximum 590 nm) and AlACR3 (487 nm) revealed four divergent positions (FIG. 2A). Most unusual were Gln213 and Ile217, which correspond, respectively, to Trp182 and Pro186 of bacteriorhodopsin and are highly conserved in the entire superfamily of microbial rhodopsins. These four unusual residues are conserved in all RubyACRs (AlACR1, AlACR1, HfACR1, and TlACR1 (FIG. 6)), but not in Chrimson (FIG. 2A), although the latter also exhibits a red-shifted absorption peak at 590 nm (Klapoetke et al., 2014).

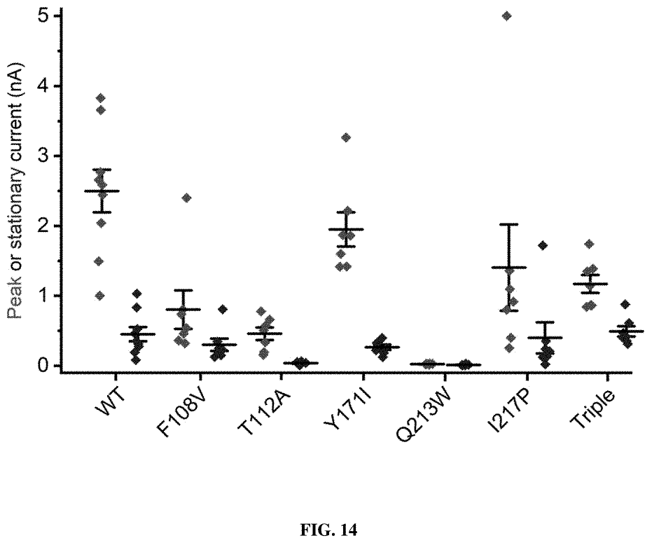

FIG. 2B shows locations of these residues according to a homology model of AlACR1. To test their possible role in color tuning, the residues were individually replaced in AlACR1 with those found in the corresponding positions in the blue-absorbing AlACR3, and the action spectra of photocurrents in the resultant mutants were measured. In the AlACR1_F108W, Y171I, and Y217P mutants the position of the main peak shifted 10, 20 and 15 nm to shorter wavelengths, respectively, whereas in the Q213W mutant it remained almost unchanged (FIGS. 2C-F). The effects of the three color-tuning mutations were additive (FIG. 2G). In Chrimson the S169A mutation caused an 18-nm red shift of the spectral maximum (Oda et al., 2018). The corresponding T112A mutation caused a similar red shift in AlACR1 (FIG. 2H) despite the differences in the retinal-binding pockets of the two wild-type proteins. FIG. 14 shows photocurrent amplitudes generated by the binding-pocket mutants.

The action spectra of photocurrents generated by all four RubyACRs exhibited an additional sharp peak at 520 nm, suggesting the presence of a second chromophore (FIG. 1B). In addition to a shift of the main (rhodopsin) spectral peak, the AlACR1_F108W, T112A, Y171I and Y217P mutations also changed the relative contribution of the 520-nm peak (its ratio to the rhodopsin peak). Notably, there was an inverse correlation between this contribution and the rhodopsin peak position (FIG. 2I) as would be expected from Førster resonance energy transfer (FRET) from the second chromophore to the retinal chromophore, because efficiency of FRET is proportional to the extent of spectral overlap between the donor and acceptor.

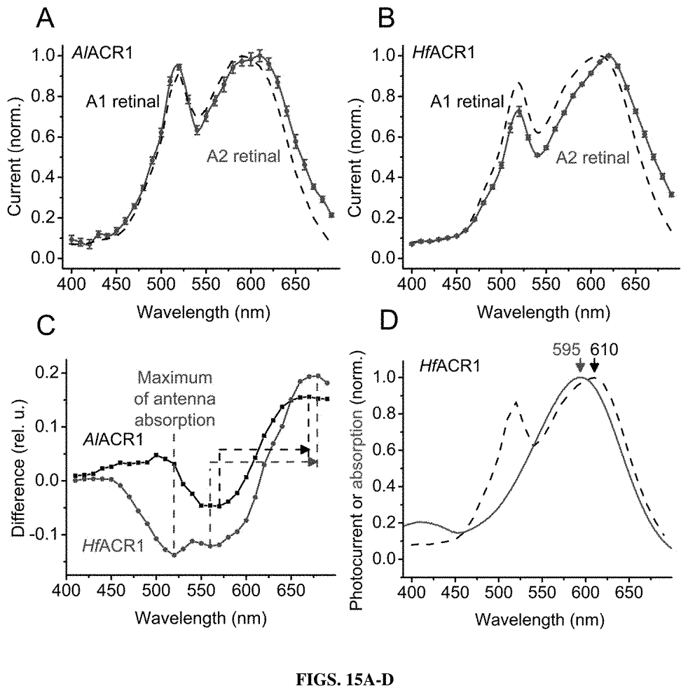

Previously, it was shown that incorporation of 3,4-dehydroretinal (A2 retinal) instead of native chromophore (A1 retinal) causes a red shift of the spectral sensitivity in several microbial rhodopsins expressed in HEK293 cells (Sineskchekov et al., 2018). Supplementation of AlACR1-expressing cells with A2 retinal led to a red shift of the rhodopsin peak of the photocurrent action spectrum as compared to that measured with A1 retinal, but did not shift the 520-nm peak (FIG. 15A). In HfACR1 (spectral maximum with A1 retinal at 610 nm), a red shift of the rhodopsin peak was observed, and also the relative amplitude of the 520-nm peak decreased without a change in its position (FIG. 15B). Both of these effects were clearly resolved in the difference (A2 retinal−A1 retinal) spectra (FIG. 15C). These observations confirmed that the 520-nm peak originated from a second chromophore, as its spectral position was not affected by the type of retinal used. Energy migration from the antenna pigment to the retinal chromophore was further confirmed by the inverse correlation of the contribution of the antenna peak to the spectrum with the wavelength of the rhodopsin peak, as in the case of the color-tuning mutants (FIG. 2I).

### Example 5—Retinal Chromophore-EYFP Interaction

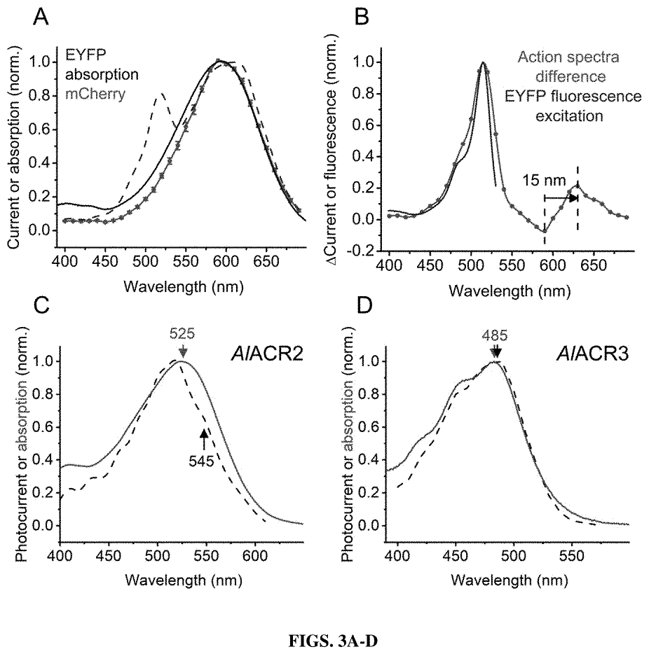

To test whether the second chromophore responsible for the 520-nm peak in the photocurrent action spectra is derived from the EYFP tag and gain more information about the spectral properties of RubyACRs, AlACR1 and HfACR1 were expressed without fluorescent tags in Pichia and the encoded proteins purified in non-denaturing detergent. The absorption spectra of detergent-purified AlACR1 (FIG. 3A, black) and HfACR1 (FIG. 15D, red) lacked the 520-nm band observed in the action spectra of the corresponding EYFP fusions. When the EYFP tag (absorption maximum at 513 nm) was replaced with mCherry tag (587 nm), the 520-nm band disappeared from the action spectrum (FIG. 3A, red). The difference between the action spectra of photocurrents generated by AlACR1 EYFP and AlACR1 mCherry matched the fluorescence excitation spectrum of EYFP (FIG. 3B). The difference between the action spectra also clearly showed that the position of the rhodopsin peak was ˜15-nm shifted to shorter wavelengths by the tag replacement (the arrow in FIG. 3B). The maximum of the action spectrum of photocurrents generated by AlACR1 mCherry was close to that of the absorption spectrum of the fluorescence tag-free protein (595 nm; FIG. 3A, red and black, respectively).

The absorption spectrum of the Pichia-expressed green-absorbing AlACR2 peaked at 525 nm (FIG. 3C, red). The action spectrum of photocurrents recorded from the AlACR2 EYFP fusion exhibited a shoulder at ˜545 nm (FIG. 3C, black). Comparison with the absorption spectrum show that this shoulder corresponds to rhodopsin absorption, whereas the main peak at 520 nm reflects energy transfer from EYFP. The absorption spectrum of the Pichia-expressed blue-absorbing AlACR3 closely matched the action spectrum of photocurrents recorded from the corresponding EYFP fusion (FIG. 3D).

### Example 6—Channel Gating in AlACR1

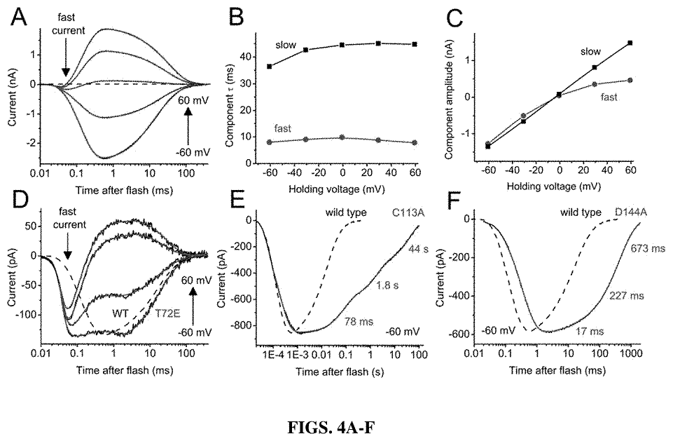

To analyze the kinetics of channel gating in AlACR1, photocurrents were recorded under single-turnover conditions using 6-ns laser flash excitation. Laser-evoked current traces could be fit with four exponentials (two for the current rise and two for the decay) (FIG. 4A). Channel opening (the time constant (τ ˜100 μs) was preceded with a fast, only weakly voltage-dependent negative peak that could be clearly resolved near the reversal voltage for channel currents (the arrow in FIG. 4A). Its rise τ was <20 μs, the lower limit of time resolution of the system. Such currents have previously been recorded from several other channelrhodopsins and attributed to a charge displacement associated with retinal chromophore isomerization, integrated by the recording circuit (Sineshchekov et al., 2013).

AlACR1 channel closing was biphasic, and both phases were faster (τ, 13±1 ms and 77±11 ms at −60 mV, n=5 cells) than those in GtACR1 (21). Moreover, in AlACR1 τ of both decay phases showed only weak (if any) voltage dependence (FIG. 4B), in contrast to GtACR1 in which the fast decay was strongly accelerated, and the slow decay slowed upon depolarization (Sineshchekov et al., 2015). The amplitude of the fast decay component in AlACR1 exhibited inward, and that of the slow decay, no rectification (FIG. 4C), again in contrast to GtACR1, in which the amplitude of the fast component showed outward, and that of the slow component, inward rectification (Sineshchekov et al., 2015).

Previously, it was found that Glu68 controls the kinetics of the fast channel closing in GtACR1 (Sineshchekov et al., 2015). The corresponding position in AlACR1 is occupied by a non-carboxylate residue, Thr72 (FIG. 6), which is very unusual for channelrhodopsins, in most of which a Glu is conserved. The T72E mutation significantly reduced laser-evoked channel currents from 2.7±0.5 nA measured in the wild type to 76±17 pA (mean±s.e.m., n=8 and 7 cells, respectively; p<0.005, Mann-Whitney test), which made the fast isomerization peak more obvious in the current trace (FIG. 4D). The current decay rate was little affected by this mutation. Cys128 from TM3 and Asp156 from TM4 form an interhelical hydrogen bond (the “DC gate”) in Chlamydomonas reinhardtii channelrhodopsin-2 (CrChR2) (Nack et al., 2010). The homologs of these residues in AlACR1 are Cys113 and Asp144. Alanine replacement of each of these residues slowed channel closing (FIGS. 4E and F). The effect was much more pronounced in the C113A mutant (the slowest decay phase was on the time scale of tens of seconds) than in the D144A mutant.

### Example 7—Unusual Photocurrents of AlACR2

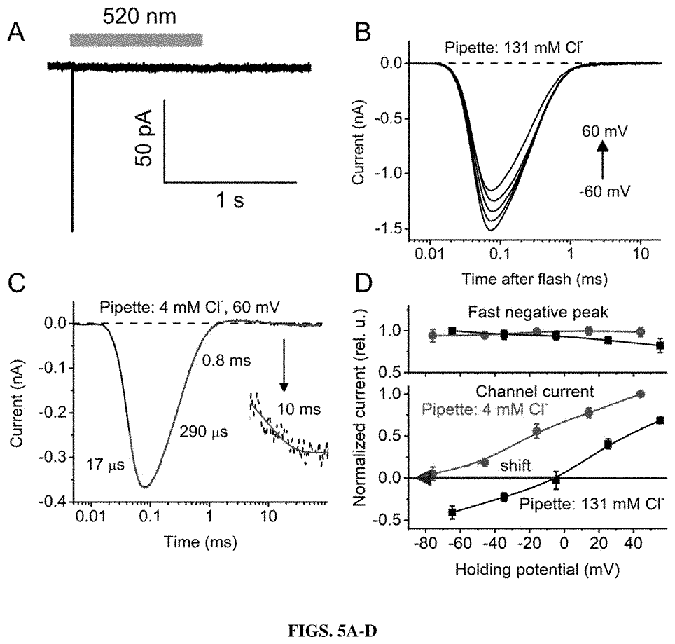

In contrast to all other channelrhodopsins studied in heterologous expression systems amenable to electrophysiological recording, photocurrents from AlACR2 were dominated by a fast negative signal (FIG. 5A). Its peak amplitude recorded in response to a 1-s pulse of continuous light at −60 mV was 103±15 pA (mean±sem, n=7 cells). It showed a very weak, in any, dependence of the holding voltage (FIG. 5B), as is typical of similar fast negative currents associated with retinal chromophore isomerization in other channelrhodopsins (Sineshchekov et al., 2013). To detect a possible contribution of passive Cl⁻ conductance in photocurrents, the Cl⁻ concentration was reduced in the pipette to 4 mM and the cells held at +60 mV. Under these conditions, passive Cl⁻ flux would be inward into the cell, producing an outward hyperpolarizing electrical current, opposite to the inward isomerization current. The laser flash-evoked current traces could be fit with four exponentials, the slowest of which had τ 10 ms (FIG. 5C), which is within the range of channel closing time in other ACRs. Next, the voltage dependence of the amplitude of this component was measured with 4 and 131 mM Cl⁻ in the pipette. FIG. 5D shows that, in contrast to the peak amplitude, the reversal potential of this slowest component followed the equilibrium potential for Cl⁻, which confirmed that AlACR2 possessed residual passive conductance for this ion.

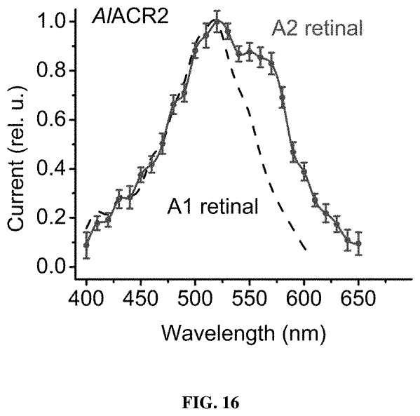

The action spectrum of the unusual photocurrents generated by the AlACR2 EYFP fusion exhibited a main peak at 520 nm and a minor shoulder at ˜540 nm (FIG. 1B, green). The position of the main peak was very close to that of EYFP absorption. To test the possibility of energy transfer from EYFP to the AlACR2 retinal chromophore, the action spectrum was measured after incubation of the cells with A2 retinal. A clear shoulder appeared at −580 nm, which can be explained by a shift of the rhodopsin peak to longer wavelengths as expected from A2 retinal binding in the chromophore pocket, whereas the peak at 520 nm remained unchanged (FIG. 16), consistent with energy transfer between EYFP and AlACR2.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
O. A. Sineshchekov, K.-H. Jung, J. L. Spudich, Two rhodopsins mediate phototaxis to low- and high-intensity light in Chlamydomonas reinhardtii. Proc. Natl. Acad. Sci. USA 99, 8689-8694 (2002).
G. Nagel, D. Ollig, M. Fuhrmann, S. Kateriya, A. M. Musti, E. Bamberg, P. Hegemann, Channelrhodopsin-1: a light-gated proton channel in green algae. Science 296, 2395-2398 (2002).
G. Nagel, T. Szellas, W. Huhn, S. Kateriya, N. Adeishvili, P. Berthold, D. Ollig, P. Hegemann, E. Bamberg, Channelrhodopsin-2, a directly light-gated cation-selective membrane channel. Proc. Natl. Acad. Sci. USA 100, 13940-13945 (2003).
E. G. Govorunova, O. A. Sineshchekov, X. Liu, R. Janz, J. L. Spudich, Natural light-gated anion channels: A family of microbial rhodopsins for advanced optogenetics. Science 349, 647-650 (2015).
E. S. Boyden, F. Zhang, E. Bamberg, G. Nagel, K. Deisseroth, Millisecond-timescale, genetically targeted optical control of neural activity. Nat. Neurosci. 8, 1263-1268 (2005).
F. Mohammad, J. C. Stewart, S. Ott, K. Chlebikova, J. Y. Chua, T. W. Koh, J. Ho, A. Claridge-Chang, Optogenetic inhibition of behavior with anion channelrhodopsins. Nat. Methods 14, 271-274 (2017).
O. A. Sineshchekov, E. G. Govorunova, H. Li, J. L. Spudich, Bacteriorhodopsin-like channelrhodopsins: Alternative mechanism for control of cation conductance. Proc. Natl. Acad. Sci. USA 114, E9512-E9519 (2017).
J. Oppermann, P. Fischer, A. Silapetere, B. Liepe, S. Rodriguez-Rozada, J. Flores-Uribe, E. Peter, A. Keidel, J. Vierock, J. Kaufmann, M. Broser, M. Luck, F. Bartl, P. Hildebrandt, J. Simon Wiegert, O. Beja, P. Hegemann, J. Wietek, MerMAIDs: a family of metagenomically discovered marine anion-conducting and intensely desensitizing channelrhodopsins. Nat. Commun. 10, 3315 (2019).
B. Leyland, S. Leu, S. Boussiba, Are Thraustochytrids algae? Fungal Biol. 121, 835-840 (2017).
V. C. Reeb, M. T. Peglar, H. S. Yoon, J. R. Bai, M. Wu, P. Shiu, J. L. Grafenberg, A. Reyes-Prieto, S. E. Rummele, J. Gross, D. Bhattacharya, Interrelationships of chromalveolates within a broadly sampled tree of photosynthetic protists. Mol. Phylogenet. Evol. 53, 202-211 (2009).
F. Burki, A. J. Roger, M. W. Brown, A. G. B. Simpson, The new tree of eukaryotes. Trends Ecol. Evol. 35, 43-55 (2020).
N. C. Klapoetke, Y. Murata, S. S. Kim, S. R. Pulver, A. Birdsey-Benson, Y. K. Cho, T. K. Morimoto, A. S. Chuong, E. J. Carpenter, Z. Tian, J. Wang, Y. Xie, Z. Yan, Y. Zhang, B. Y. Chow, B. Surek, M. Melkonian, V. Jayaraman, M. Constantine-Paton, G. K. Wong, E. S. Boyden, Independent optical excitation of distinct neural populations. Nat. Methods 11, 338-346 (2014).
J. Wietek, S. Rodriguez-Rozada, J. Tutas, F. Tenedini, C. Grimm, T. G. Oertner, P. Soba, P. Hegemann, J. S. Wiegert, Anion-conducting channelrhodopsins with tuned spectra and modified kinetics engineered for optogenetic manipulation of behavior. Sci. Rep. 7, 14957 (2017).
E. G. Govorunova, O. A. Sineshchekov, E. M. Rodarte, R. Janz, O. Morelle, M. Melkonian, G. K.-S. Wong, J. L. Spudich, The expanding family of natural anion channelrhodopsins reveals large variations in kinetics, conductance, and spectral sensitivity. Sci. Rep. 7, 43358 (2017).
E. G. Govorunova, O. A. Sineshchekov, R. Hemmati, R. Janz, O. Morelle, M. Melkonian, G. K. S. Wong, J. L. Spudich, Extending the time domain of neuronal silencing with cryptophyte anion channelrhodopsins. eNeuro 27, ENEURO.0174-0118.2018 (2018).
A. Rozenberg, J. Oppermann, J. Wietek, R. G. F. Lahore, R.-A. Sandaa, G. Bratbak, P. Hegemann, O. Béjà, Lateral gene transfer of anion-conducting channelrhodopsins between green algae and giant viruses. bioRxiv, 2020.2004.2015.042127 (2020).
S. Mukherjee, P. Hegemann, M. Broser, Enzymerhodopsins: novel photoregulated catalysts for optogenetics. Curr. Opin. Struct. Biol. 57, 118-126 (2019).
K. Oda, J. Vierock, S. Oishi, S. Rodriguez-Rozada, R. Taniguchi, K. Yamashita, J. S. Wiegert, T. Nishizawa, P. Hegemann, O. Nureki, Crystal structure of the red light-activated channelrhodopsin Chrimson. Nat. Commun. 9, 3949 (2018).
O. A. Sineshchekov, E. G. Govorunova, J. Wang, J. L. Spudich, Enhancement of the long-wavelength sensitivity of optogenetic microbial rhodopsins by 3,4-dehydroretinal. Biochemistry 51, 4499-4506 (2012).
O. A. Sineshchekov, E. G. Govorunova, J. Wang, H. Li, J. L. Spudich, Intramolecular proton transfer in channelrhodopsins. Biophys. J. 104, 807-817 (2013).
O. A. Sineshchekov, E. G. Govorunova, H. Li, J. L. Spudich, Gating mechanisms of a natural anion channelrhodopsin. Proc. Natl. Acad. Sci. USA 112, 14236-14241 (2015).
M. Nack, I. Radu, M. Gossing, C. Bamann, E. Bamberg, G. F. von Mollard, J. Heberle, The DC gate in channelrhodopsin-2: crucial hydrogen bonding interaction between C128 and D156. Photochem. Photobiol. Sci. 9, 194-198 (2010).
A. S. Chuong, M. L. Miri, V. Busskamp, G. A. Matthews, L. C. Acker, A. T. Sorensen, A. Young, N. C. Klapoetke, M. A. Henninger, S. B. Kodandaramaiah, M. Ogawa, S. B. Ramanlal, R. C. Bandler, B. D. Allen, C. R. Forest, B. Y. Chow, X. Han, Y. Lin, K. M. Tye, B. Roska, J. A. Cardin, E. S. Boyden, Noninvasive optical inhibition with a red-shifted microbial rhodopsin. Nat. Neurosci. 17, 1123-1129 (2014).
S. P. Balashov, J. K. Lanyi, Xanthorhodopsin: Proton pump with a carotenoid antenna. Cell. Mol.

Life Sci. 64, 2323-2328 (2007).
S. P. Balashov, E. S. Imasheva, A. R. Choi, K. H. Jung, S. Liaaen-Jensen, J. K. Lanyi, Reconstitution of gloeobacter rhodopsin with echinenone: role of the 4-keto group. Biochemistry 49, 9792-9799 (2010).
H. Luecke, B. Schobert, J. Stagno, E. S. Imasheva, J. M. Wang, S. P. Balashov, J. K. Lanyi, Crystallographic structure of xanthorhodopsin, the light-driven proton pump with a dual chromophore. Proc. Natl. Acad. Sci. USA 105, 16561-16565 (2008).
E. S. Imasheva, S. P. Balashov, J. M. Wang, E. Smolensky, M. Sheves, J. K. Lanyi, Chromophore interaction in xanthorhodopsin—retinal dependence of salinixanthin binding. Photochem. Photobiol. 84, 977-984 (2008).
T. Polívka, S. P. Balashov, P. Chábera, E. S. Imasheva, A. Yartsev, V. Sundstrom, J. K. Lanyi, Femtosecond carotenoid to retinal energy transfer in xanthorhodopsin. Biophys. J. 96, 2268-2277 (2009).
Y. Gong, M. J. Wagner, J. Zhong Li, M. J. Schnitzer, Imaging neural spiking in brain tissue using FRET-opsin protein voltage sensors. Nat. Commun. 5, 3674 (2014).
P. Zou, Y. Zhao, A. D. Douglass, D. R. Hochbaum, D. Brinks, C. A. Werley, D. J. Harrison, R. E. Campbell, A. E. Cohen, Bright and fast multicoloured voltage reporters via electrochromic FRET. Nat. Commun. 5, 4625 (2014).
N. Azimi Hashemi, A. C. F. Bergs, C. Schuler, A. R. Scheiwe, W. Steuer Costa, M. Bach, J. F. Liewald, A. Gottschalk, Rhodopsin-based voltage imaging tools for use in muscles and neurons of Caenorhabditis elegans. Proc. Natl. Acad. Sci. USA, (2019).
K. Fujimoto, S. Hayashi, J. Y. Hasegawa, H. Nakatsuji, Theoretical studies on the color-tuning mechanism in retinal proteins. J. Chem. Theory Comput. 3, 605-618 (2007).
F. Melaccio, N. Ferre, M. Olivucci, Quantum chemical modeling of rhodopsin mutants displaying switchable colors. Phys. Chem. Chem. Phys. 14, 12485-12495 (2012).
K. Katayama, S. Sekharan, Y. Sudo (2015) Color tuning in retinylidene proteins. Optogenetics: Light-Sensing Proteins and their Applications, eds Yawo H, Kandori H, & Koizumi A (Springer Japan, Tokyo), pp 89-109.
S. Gozem, H. L. Luk, I. Schapiro, M. Olivucci, Theory and simulation of the ultrafast double-bond isomerization of biological chromophores. Chem. Rev. 117, 13502-13565 (2017).
J. Hufen, M. Sugihara, V. Buss, How the counterion affects ground- and excited-state properties of the rhodopsin chromophore. J. Phys. Chem. B 108, 20419-20426 (2004).
M. Karasuyama, K. Inoue, R. Nakamura, H. Kandori, I. Takeuchi, Understanding colour tuning rules and predicting absorption wavelengths of microbial rhodopsins by data-driven machine-learning approach. Sci. Rep. 8, 15580 (2018).
T. Marti, S. J. Rosselet, H. Otto, M. P. Heyn, H. G. Khorana, The retinylidene Schiff base counterion in bacteriorhodopsin. J. Biol. Chem. 266, 18674-18683 (1991).
K. Inoue, S. P. Tsunoda, M. Singh, S. Tomida, S. Hososhima, M. Konno, R. Nakamura, H. Watanabe, P. A. Bulzu, H. L. Banciu, A.-S. Andrei, T. Uchihashi, R. Ghai, O. Béjà, H. Kandori, Schizorhodopsins: A novel family of rhodopsins from Asgard archaea that function as light-driven inward H+ pumps. Sci. Adv. 6, eaaz2441 (2020).
A. Harris, M. Lazaratos, M. Siemers, E. Watt, A. Hoang, S. Tomida, L. Schubert, M. Saita, J. Heberle, Y. Furutani, H. Kandori, A. N. Bondar, L. S. Brown, Mechanism of inward proton transport in an antarctic microbial rhodopsin. J. Phys. Chem. B, in press (2020).
K. Inoue, M. Del Carmen Marin, S. Tomida, R. Nakamura, Y. Nakajima, M. Olivucci, H. Kandori, Red-shifting mutation of light-driven sodium-pump rhodopsin. Nat. Commun. 10, 1993 (2019).
D. Man, W. Wang, G. Sabehi, L. Aravind, A. F. Post, R. Massana, E. N. Spudich, J. L. Spudich, O. Béjà, Diversification and spectral tuning in marine proteorhodopsins. EMBO J. 22, 1725-1731 (2003).
L. Peperzak, F. Colijn, E. G. Vrieling, W. W. C. Gieskes, J. C. H. Peeters, Observations of flagellates in colonies of Phaeocystis globosa (Prymnesiophyceae): a hypothesis for their position in the life cycle. J. Plankton Res. 22, 2181-2203 (2000).
R. M. Bennett, D. Honda, G. W. Beakes, M. Thines (2017) Labyrinthulomycota. Handbook of the Protists, eds Archibald J, Simpson A, & Slamovits C (Springer, Cham), pp 507-542.
I. Moro, E. Negrisolo, A. Callegaro, C. Andreoli, Aplanochytrium stocchinoi: a new Labyrinthulomycota from the southern ocean (Ross Sea, Antarctica). Protist 154, 331-340 (2003).
J. Trifinopoulos, L. T. Nguyen, A. von Haeseler, B. Q. Minh, W-IQ-TREE: a fast online phylogenetic tool for maximum likelihood analysis. Nucleic Acids Res. 44, W232-235 (2016).
I. Letunic, P. Bork, Interactive Tree Of Life (iTOL) v4: recent updates and new developments. Nucleic Acids Res. 47, W256-W259 (2019).
J. Yang, R. Yan, A. Roy, D. Xu, J. Poisson, Y. Zhang, The I-TASSER suite: Protein structure and function prediction. Nat. Methods 12, 7-8 (2015).
S. Ovchinnikov, H. Park, D. E. Kim, F. DiMaio, D. Baker, Protein structure prediction using Rosetta in CASP12. Proteins 86 Suppl 1, 113-121 (2018).
J. Haas, R. Gumienny, A. Barbato, F. Ackermann, G. Tauriello, M. Bertoni, G. Studer, A. Smolinski, T. Schwede, Introducing “best single template” models as reference baseline for the Continuous Automated Model Evaluation (CAMEO). Proteins 87, 1378-1387 (2019).
D. M. Nikolaev, A. A. Shtyrov, M. S. Panov, A. Jamal, O. B. Chakchir, V. A. Kochemirovsky, M. Olivucci, M. N. Ryazantsev, A comparative study of modern homology modeling algorithms for rhodopsin structure prediction. ACS Omega 3, 7555-7566 (2018).
P. H. Barry, JPCalc, a software package for calculating liquid junction potential corrections in patch-clamp, intracellular, epithelial and bilayer measurements and for correcting junction potential measurements. J. Neurosci. Methods 51, 107-116 (1994).
R. B. Dell, S. Holleran, R. Ramakrishnan, Sample size determination. ILAR J. 43, 207-213 (2002).
A. Berndt, S. Y. Lee, C. Ramakrishnan, K. Deisseroth, Science 344, 420 (2014).
A. S. Chuong et al., Nat. Neurosci. 17, 1123 (2014).
B. A. Curtis et al., Nature 492, 59 (2012).
B. Y. Chow et al., Nature 463, 98 (2010).
B. Y. Chow, E. S. Boyden, Sci. Transl. Med. 5, 177ps5 (2013).
D. Gradmann, A. Berndt, F. Schneider, P. Hegemann, Biophys. J. 101, 1057 (2011).
Daniel et al. ATGme: Open-source web application for rare codon identification and custom DNA sequence optimization. BMC Bioinformatics, 16:303, 2015.
E. G. Govorunova, O. A. Sineshchekov, H. Li, R. Janz, J. L. Spudich, J. Biol. Chem. 288, 29911 (2013).
F. Zhang et al., Cell 147, 1446 (2011).
F. Zhang et al., Nature 446, 633 (2007).
G. Nagel et al., Proc. Natl. Acad. Sci. USA 100, 13940 (2003).
G. Nagel et al., Science 296, 2395 (2002).
Govorunova E G et al. Photochem Photobiol. 2015.
Govorunova E G et al. Science. 349(6248): 647-50, 2015.
Grote et al. JCat: a novel tool to adapt codon usage of a target gene to its potential expression host. Nucleic Acids Research, 33: W526-W531, 2005.
J. L. Spudich, O. A. Sineshchekov, E. G. Govorunova, Biochem. Biophys. Acta 1837, 546 (2014).
J. Wietek et al., Science 344, 409 (2014).
J. Y. Lin, P. M. Knutsen, A. Muller, D. Kleinfeld, R. Y. Tsien, Nat. Neurosci. 16, 1499 (2013).
K. Deisseroth, Nat. Methods 8, 26 (2011).
K. Eisenhauer et al., J. Biol. Chem. 287, 6904 (2012).
K. Ruffert et al., Biochem. Biophys. Res. Commun. 410, 737 (2011).
M. Kolbe, H. Besir, L. O. Essen, D. Oesterhelt, Science 288, 1390 (2000).
N. C. Klapoetke et al., Nat. Methods 11, 338 (2014).
O. A. Sineshchekov et al., Biophys. J. 89, 4310 (2005).
O. A. Sineshchekov, K.-H. Jung, J. L. Spudich, Proc. Natl. Acad. Sci. USA 99, 8689 (2002).
O. P. Ernst et al., Chem. Rev. 114, 126 (2014).
P. Bregestovski, T. Waseem, M. Mukhtarov, Front. Mol. Neurosci. 2, 15 (2009).
S. J. Husson et al., PLoS One 7, e40937 (2012).
Sineshchekov O A et al. Proc Natl Acad Sci USA. 112 (46):14236-41, 2015.
T. J. Jentsch, V. Stein, F. Weinreich, A. A. Zdebik, Physiol Rev. 0.82, 503 (2002).
U.S. Patent Application No. 62/261,821.
V. Gradinaru et al., Cell 141, 154 (2010).
V. Gradinaru, K. R. Thompson, K. Deisseroth, Brain Cell Biol. 36, 129 (2008).
X. Han, E. S. Boyden, PLoS One 2, e299 (2007).

1. A recombinant nucleic acid operatively linked to a heterologous promoter sequence, said recombinant nucleic acid comprising:
(i) a sequence encoding an anion-conducting channelrhodopsin having a polypeptide sequence that is at least about 90% identical to the polypeptide sequence encoded by SEQ ID NO: 1-3, SEQ ID NO: 5-7, SEQ ID NO: 9-11, or SEQ ID NO: 13-15;
(ii) a sequence encoding an anion-conducting channelrhodopsin having a polypeptide sequence that is at least about 90% identical to a sequence selected from SEQ ID NO: 4, SEQ ID NO: 8, SEQ ID NO: 12, or SEQ ID NO: 16; or
(iii) a sequence that is at least about 90% identical to a sequence selected from the group consisting of SEQ ID NO: 1-3, SEQ ID NO: 5-7, SEQ ID NO: 9-11, or SEQ ID NO: 13-15 and encodes an anion-conducting channelrhodopsin.

2. The recombinant nucleic acid of claim 1, wherein it comprises an expression vector.

3. A recombinant host cell comprising a recombinant nucleic acid of claim 1.

4. The recombinant host cell of claim 3, wherein said host cell is an isolated human cell.

5. The recombinant host cell of claim 3, wherein said host cell is a non-human mammalian cell.

6-11. (canceled)

12. A method of membrane potential photocontrol, including hyperpolarization of a cell in a subject suffering from a neuron mediated disorder, said method comprising:
(a) delivering to a cell of said subject an expression vector encoding:
(i) a sequence encoding an anion-conducting channelrhodopsin having a polypeptide sequence that is at least about 90% identical to the polypeptide sequence encoded by SEQ ID NO: 1-3, SEQ ID NO: 5-7, SEQ ID NO: 9-11, or SEQ ID NO: 13-15;
(ii) a sequence encoding an anion-conducting channelrhodopsin having a polypeptide sequence that is at least about 90% identical to a sequence selected from SEQ ID NO: 4, SEQ ID NO: 8, SEQ ID NO: 12, or SEQ ID NO: 16; or
(iii) a sequence that is at least about 90% identical to a sequence selected from the group consisting of SEQ ID NO: 1-3, SEQ ID NO: 5-7, SEQ ID NO: 9-11, or SEQ ID NO: 13-15 and encodes an anion-conducting channelrhodopsin; and
(b) expressing said vector in said cell, wherein the expressed rhodopsin silences the signal from said neuron or in the case of neurons with high cytoplasmic chloride concentration, wherein the expressed rhodopsin depolarizes the plasma membrane.

13. The method of claim 12, wherein said method comprises:
(a) delivering to the cell of said subject an expression vector comprising a polynucleotide sequence at least about 90% identical to a sequence selected from the group consisting of SEQ ID NO: 1-3, SEQ ID NO: 5-7, SEQ ID NO: 9-11, or SEQ ID NO: 13-15, which encodes an anion-conducting rhodopsin domain expressible in said cell; and
(b) expressing said vector in said cell, wherein the expressed rhodopsin silences the signal from said neuron, or in the case of neurons with high cytoplasmic chloride concentration, wherein the expressed rhodopsin depolarizes the plasma membrane.

14. The method of claim 12, wherein said method comprises:
(a) delivering to the cell of said subject an expression vector that encodes a rhodopsin domain, said vector comprising an open reading frame having a nucleic acid sequence at least about 90% identical to a sequence selected from the group consisting of SEQ ID NO: 1-3, SEQ ID NO: 5-7, SEQ ID NO: 9-11, or SEQ ID NO: 13-15, operatively linked to a promoter sequence; and
(b) expressing said vector in said cell, wherein the expressed rhodopsin renders a high level of membrane potential in said cell.

15. The method of claim 12, wherein
said potential photocontrol restores photosensitivity to a retina of a subject suffering from vision loss or blindness.

16-27. (canceled)

28. An isolated nucleic acid molecule comprising a sequence encoding an anion-conducting channelrhodopsin having a sequence at least about 90% identical to a sequence according to SEQ ID NO: 1-3, SEQ ID NO: 5-7, SEQ ID NO: 9-11, or SEQ ID NO: 13-15.

29. The isolated nucleic acid molecule of claim 28, comprising a sequence having a sequence at least about 95% identical to a sequence according to SEQ ID NO: 1-3, SEQ ID NO: 5-7, SEQ ID NO: 9-11, or SEQ ID NO: 13-15.

30. The isolated nucleic acid molecule of claim 28, where the nucleic acid is a DNA.

31. The isolated nucleic acid molecule of claim 28, where the nucleic acid is an RNA.

32. The isolated nucleic acid molecule of claim 31, where the nucleic acid is an mRNA.

33. An expression vector comprising a nucleic acid molecule according to claim 28.

34. A recombinant host cell comprising a nucleic acid of claim 28.

35. The recombinant host cell of claim 34, wherein said host cell is an isolated human cell.

36. The recombinant host cell of claim 34, wherein said host cell is a non-human mammalian cell.

37-40. (canceled)

41. The recombinant host cell of claim 34, wherein said host cell is an isolated neuronal cell.

42. The recombinant host cell of claim 34, wherein said host cell is an isolated electrically active cell.

43-58. (canceled)


SEQUENCE LISTING (23 sequences)

SEQ ID NO: 1 - DNA, 900 bp, Hondaea fermentalgiana FCC1311
atgagctacaacgagaccgtgtggagggaggacttcgcacaggtgctgctggcccggccccagaacagcggcgtggtgagccagggagtggcaggagcaaggctgtgcgtgtttagctgttccatcgccctgctgtgcttctatacctttcagtggaggatcggccgctgtaacctgcaccccatctacgtgcagttcgtgacattttgcgtgtatatcctgtcttgtttcggcaccacagattttctgtacatcccagtggtggaccacgcaggagtgaccttcgatctggccatcggcagatatctgttttggatgatgacatgccctgtgatcatcagcaacgtgaatatcctgctgaacatcatgacccctgacgatatcgagctgccacaggtgacattcatgatggtgaaggacatcgtgatgatcgcctttggcgtgctggccgccatgcagcagaacatcaccctgaagtggatcttcaatctggcctcctacttcgtgtgcttttggctggtggccgatctgatcgtgctgatgaatgagaagaagaagttcttcatggtgtatgagcggtgctgggagtggatcttcgccgtgatgatctacttcttttgtgcccagagcctgttcatctttctgtatgccgtgggccccccttgttttaacatcatgggcccagacggcgataggatcggccactccgtgggcgacctgttcgccaagaatctgtttggcttctttgcctggtacgtgcgcttcgtggtgctggacccccacgtgaagcggatcagaggagcaagggccgtgtctccatatcagaccaggccagtgaccgcaacagaccagacatccaaccgggtgtcttttctgggcagaaagaccaatacaaagaag

SEQ ID NO: 2 - DNA, 900 bp, Artificial Sequence
  Synthetic polynucleotide
atgagctacaacgagaccgtgtggcgcgaggacttcgcccaggtgctgctggcccgcccccagaacagcggcgtggtgagccagggcgtggccggcgcccgcctgtgcgtgttcagctgcagcatcgccctgctgtgcttctacaccttccagtggcgcatcggccgctgcaacctgcaccccatctacgtgcagttcgtgaccttctgcgtgtacatcctgagctgcttcggcaccaccgacttcctgtacatccccgtggtggaccacgccggcgtgaccttcgacctggccatcggccgctacctgttctggatgatgacctgccccgtgatcatcagcaacgtgaacatcctgctgaacatcatgacccccgacgacatcgagctgccccaggtgaccttcatgatggtgaaggacatcgtgatgatcgccttcggcgtgctggccgccatgcagcagaacatcaccctgaagtggatcttcaacctggccagctacttcgtgtgcttctggctggtggccgacctgatcgtgctgatgaacgagaagaagaagttcttcatggtgtacgagcgctgctgggagtggatcttcgccgtgatgatctacttcttctgcgcccagagcctgttcatcttcctgtacgccgtgggccccccctgcttcaacatcatgggccccgacggcgaccgcatcggccacagcgtgggcgacctgttcgccaagaacctgttcggcttcttcgcctggtacgtgcgcttcgtggtgctggacccccacgtgaagcgcatccgcggcgcccgcgccgtgagcccctaccagacccgccccgtgaccgccaccgaccagaccagcaaccgcgtgagcttcctgggccgcaagaccaacaccaagaag

SEQ ID NO: 3 - DNA, 900 bp, Artificial Sequence
  Synthetic polynucleotide
atgagctacaacgagaccgtgtggagagaggacttcgcccaggtgctgctggccagaccccagaacagcggcgtggtgagccagggcgtggccggcgccagactgtgcgtgttcagctgcagcatcgccctgctgtgcttctacaccttccagtggagaatcggcagatgcaacctgcaccccatctacgtgcagttcgtgaccttctgcgtgtacatcctgagctgcttcggcaccaccgacttcctgtacatccccgtggtggaccacgccggcgtgaccttcgacctggccatcggcagatacctgttctggatgatgacctgccccgtgatcatcagcaacgtgaacatcctgctgaacatcatgacccccgacgacatcgagctgccccaggtgaccttcatgatggtgaaggacatcgtgatgatcgccttcggcgtgctggccgccatgcagcagaacatcaccctgaagtggatcttcaacctggccagctacttcgtgtgcttctggctggtggccgacctgatcgtgctgatgaacgagaagaagaagttcttcatggtgtacgagagatgctgggagtggatcttcgccgtgatgatctacttcttctgcgcccagagcctgttcatcttcctgtacgccgtgggccccccctgcttcaacatcatgggccccgacggcgacagaatcggccacagcgtgggcgacctgttcgccaagaacctgttcggcttcttcgcctggtacgtgagattcgtggtgctggacccccacgtgaagagaatcagaggcgccagagccgtgagcccctaccagaccagacccgtgaccgccaccgaccagaccagcaacagagtgagcttcctgggcagaaagaccaacaccaagaag

SEQ ID NO: 4 - protein, 300 aa, Hondaea fermentalgiana FCC1311
MSYNETVWREDFAQVLLARPQNSGVVSQGVAGARLCVFSCSIALLCFYTFQWRIGRCNLHPIYVQFVTFCVYILSCFGTTDFLYIPVVDHAGVTFDLAIGRYLFWMMTCPVIISNVNILLNIMTPDDIELPQVTFMMVKDIVMIAFGVLAAMQQNITLKWIFNLASYFVCFWLVADLIVLMNEKKKFFMVYERCWEWIFAVMIYFFCAQSLFIFLYAVGPPCFNIMGPDGDRIGHSVGDLFAKNLFGFFAWYVRFVVLDPHVKRIRGARAVSPYQTRPVTATDQTSNRVSFLGRKTNTKK

SEQ ID NO: 5 - DNA, 810 bp, Aurantiochytrium sp. KH105
atgtcttacaacgagagcgtgtggagggaggacttcgcacaggtgctgctggcaaggcctaagggctccggcgtggtgtctcagggagtggcaggagcaaggctgtgcgtgtttagctgttccatcgccctgctgtgcttctatgcctttcagtggaagatcggcagatgtaatctgcaccccatctacgtgcagttcgtgaccttttgcgtgtatatcctgtcctgtttcggcaccacagattttctgtacatcccagtggtggaccacgcaggagtgaccttcgatctggccatcggccggtatctgttttggatgatgacatgccctgtgatcatctctaacgtgaatatcctgctgaacatcatgacccctgacgatatcgagctgccacaggtgacattcatgatggtgaaggacatcgtgatgatcgcctttggcgtgctggccgccatgcagcagaacatcacactgaagtggatcttcaatctggccagctacttcgtgtgcttttggctggtggccgatctgatcgtgctgatgaatgagaagaagaagttcttcatggtgtatgagagatgctgggagtggatcttcgccgtgatgatctacttcttttgtgcccagtctctgttcatctttctgtatgccgtgggccccccttgttttaacatcatgggcccagacggcgataggatcggccacagcgtgggcgacctgttcgccaagaatctgtttggcttctttgcctggtacgtgcgcttcgtggtgctggacccccacgtgaagcggatcagaggagcaagggcc

SEQ ID NO: 6 - DNA, 810 bp, Artificial Sequence
  Synthetic polynucleotide
atgagctacaacgagagcgtgtggcgcgaggacttcgcccaggtgctgctggcccgccccaagggcagcggcgtggtgagccagggcgtggccggcgcccgcctgtgcgtgttcagctgcagcatcgccctgctgtgcttctacgccttccagtggaagatcggccgctgcaacctgcaccccatctacgtgcagttcgtgaccttctgcgtgtacatcctgagctgcttcggcaccaccgacttcctgtacatccccgtggtggaccacgccggcgtgaccttcgacctggccatcggccgctacctgttctggatgatgacctgccccgtgatcatcagcaacgtgaacatcctgctgaacatcatgacccccgacgacatcgagctgccccaggtgaccttcatgatggtgaaggacatcgtgatgatcgccttcggcgtgctggccgccatgcagcagaacatcaccctgaagtggatcttcaacctggccagctacttcgtgtgcttctggctggtggccgacctgatcgtgctgatgaacgagaagaagaagttcttcatggtgtacgagcgctgctgggagtggatcttcgccgtgatgatctacttcttctgcgcccagagcctgttcatcttcctgtacgccgtgggccccccctgcttcaacatcatgggccccgacggcgaccgcatcggccacagcgtgggcgacctgttcgccaagaacctgttcggcttcttcgcctggtacgtgcgcttcgtggtgctggacccccacgtgaagcgcatccgcggcgcccgcgcc

SEQ ID NO: 7 - DNA, 810 bp, Artificial Sequence
  Synthetic polynucleotide
atgagctacaacgagagcgtgtggagagaggacttcgcccaggtgctgctggccagacccaagggcagcggcgtggtgagccagggcgtggccggcgccagactgtgcgtgttcagctgcagcatcgccctgctgtgcttctacgccttccagtggaagatcggcagatgcaacctgcaccccatctacgtgcagttcgtgaccttctgcgtgtacatcctgagctgcttcggcaccaccgacttcctgtacatccccgtggtggaccacgccggcgtgaccttcgacctggccatcggcagatacctgttctggatgatgacctgccccgtgatcatcagcaacgtgaacatcctgctgaacatcatgacccccgacgacatcgagctgccccaggtgaccttcatgatggtgaaggacatcgtgatgatcgccttcggcgtgctggccgccatgcagcagaacatcaccctgaagtggatcttcaacctggccagctacttcgtgtgcttctggctggtggccgacctgatcgtgctgatgaacgagaagaagaagttcttcatggtgtacgagagatgctgggagtggatcttcgccgtgatgatctacttcttctgcgcccagagcctgttcatcttcctgtacgccgtgggccccccctgcttcaacatcatgggccccgacggcgacagaatcggccacagcgtgggcgacctgttcgccaagaacctgttcggcttcttcgcctggtacgtgagattcgtggtgctggacccccacgtgaagagaatcagaggcgccagagcc

SEQ ID NO: 8 - protein, 270 aa, Aurantiochytrium sp. KH105
MSYNESVWREDFAQVLLARPKGSGVVSQGVAGARLCVFSCSIALLCFYAFQWKIGRCNLHPIYVQFVTFCVYILSCFGTTDFLYIPVVDHAGVTFDLAIGRYLFWMMTCPVIISNVNILLNIMTPDDIELPQVTFMMVKDIVMIAFGVLAAMQQNITLKWIFNLASYFVCFWLVADLIVLMNEKKKFFMVYERCWEWIFAVMIYFFCAQSLFIFLYAVGPPCFNIMGPDGDRIGHSVGDLFAKNLFGFFAWYVRFVVLDPHVKRIRGARA

SEQ ID NO: 9 - DNA, 900 bp, Aurantiochytrium limacinum ATCC MYA-1381
atggaggccatggccatgagcgagcacgagctgtggaggagagagtttatcagggtgctgcagacccagcccaccacagacgagacagtgtctcagggagtggcaggagcaagcctgtgcgtgttcagctgttccaccgccctgatctgcttttacggcttccagtataggaacggcagatgtaatctgcaccccatctacgtgcagtttgtgacattctgcgtgtattctctggcctgttttggcaccacagatttcctgtacatccctatcgagaaccacgccggcgacgtgtttgatctggccatcggcagatatctgttctggatgatgacctgccctgtgatcatcagcaacgtgaatatcctgctgaatctgatgaccccagacgatctggagctgggccacgtgaccttcatgatggtgaaggacatcgtgatgatcgccttcggcgtgctggccgccatgcagaagaacgtgaccctgaagtggatctttaatctggcctcctacttcgtgtgcttctggctggtggtggatctgctggccatgatccgggagaagaagaagcacttcatcgtgcacgagaggtgctgggagtgggtgctggccgtgatgacatacttctttttcgcccagagcctgtttatcttcctgtatgcctttggccccccttgtttcaacctgatgggcccagacggcgataagatcggccactccgtgggcgacctgtttgccaagaatctgttcggctttttcgcctggtacgtgaggtttgtggtgctggagcccttcgtgaagaggatcaggagcgccagggcagtgaccccatatcagaggggctctgatgcaatggcaccacacaagatgaggaacgtgtctttcctgaatagaaagaca

SEQ ID NO: 10 - DNA, 900 bp, Artificial Sequence
  Synthetic polynucleotide
atggaggccatggccatgagcgagcacgagctgtggcgccgcgagttcatccgcgtgctgcagacccagcccaccaccgacgagaccgtgagccagggcgtggccggcgccagcctgtgcgtgttcagctgcagcaccgccctgatctgcttctacggcttccagtaccgcaacggccgctgcaacctgcaccccatctacgtgcagttcgtgaccttctgcgtgtacagcctggcctgcttcggcaccaccgacttcctgtacatccccatcgagaaccacgccggcgacgtgttcgacctggccatcggccgctacctgttctggatgatgacctgccccgtgatcatcagcaacgtgaacatcctgctgaacctgatgacccccgacgacctggagctgggccacgtgaccttcatgatggtgaaggacatcgtgatgatcgccttcggcgtgctggccgccatgcagaagaacgtgaccctgaagtggatcttcaacctggccagctacttcgtgtgcttctggctggtggtggacctgctggccatgatccgcgagaagaagaagcacttcatcgtgcacgagcgctgctgggagtgggtgctggccgtgatgacctacttcttcttcgcccagagcctgttcatcttcctgtacgccttcggccccccctgcttcaacctgatgggccccgacggcgacaagatcggccacagcgtgggcgacctgttcgccaagaacctgttcggcttcttcgcctggtacgtgcgcttcgtggtgctggagcccttcgtgaagcgcatccgcagcgcccgcgccgtgaccccctaccagcgcggcagcgacgccatggccccccacaagatgcgcaacgtgagcttcctgaaccgcaagacc

SEQ ID NO: 11 - DNA, 900 bp, Artificial Sequence
  Synthetic polynucleotide
atggaggccatggccatgagcgagcacgagctgtggagaagagagttcatcagagtgctgcagacccagcccaccaccgacgagaccgtgagccagggcgtggccggcgccagcctgtgcgtgttcagctgcagcaccgccctgatctgcttctacggcttccagtacagaaacggcagatgcaacctgcaccccatctacgtgcagttcgtgaccttctgcgtgtacagcctggcctgcttcggcaccaccgacttcctgtacatccccatcgagaaccacgccggcgacgtgttcgacctggccatcggcagatacctgttctggatgatgacctgccccgtgatcatcagcaacgtgaacatcctgctgaacctgatgacccccgacgacctggagctgggccacgtgaccttcatgatggtgaaggacatcgtgatgatcgccttcggcgtgctggccgccatgcagaagaacgtgaccctgaagtggatcttcaacctggccagctacttcgtgtgcttctggctggtggtggacctgctggccatgatcagagagaagaagaagcacttcatcgtgcacgagagatgctgggagtgggtgctggccgtgatgacctacttcttcttcgcccagagcctgttcatcttcctgtacgccttcggccccccctgcttcaacctgatgggccccgacggcgacaagatcggccacagcgtgggcgacctgttcgccaagaacctgttcggcttcttcgcctggtacgtgagattcgtggtgctggagcccttcgtgaagagaatcagaagcgccagagccgtgaccccctaccagagaggcagcgacgccatggccccccacaagatgagaaacgtgagcttcctgaacagaaagacc

SEQ ID NO: 12 - protein, 300 aa, Aurantiochytrium limacinum ATCC MYA-1381
MEAMAMSEHELWRREFIRVLQTQPTTDETVSQGVAGASLCVFSCSTALICFYGFQYRNGRCNLHPIYVQFVTFCVYSLACFGTTDFLYIPIENHAGDVFDLAIGRYLFWMMTCPVIISNVNILLNLMTPDDLELGHVTFMMVKDIVMIAFGVLAAMQKNVTLKWIFNLASYFVCFWLVVDLLAMIREKKKHFIVHERCWEWVLAVMTYFFFAQSLFIFLYAFGPPCFNLMGPDGDKIGHSVGDLFAKNLFGFFAWYVRFVVLEPFVKRIRSARAVTPYQRGSDAMAPHKMRNVSFLNRKT

SEQ ID NO: 13 - DNA, 810 bp, Thraustochytrium sp. ATCC 26185
atggaccctctgaacatggatgagcacgaggcatggaggagagacttcgcaaggctgctgcaggagcagcctgagagctccgtgccacagggagtggcaggagccagactgtgcgtgttttcttgtagcatcgccctgatctgcttctacggcgtgcagtatcgggccggcagatgtaacctgcacccactgtacgtgcagtttgtgaccttctgcgtgtattccctgtcttgttttggcaccacagacttcctgtacatcccaatccagaatcacgccggcgagacctttgatctggccgtgggcaggtatctgttctggatgatgacatgccccgtgatcatctccaacgtgaatatcctgctgaacctgatgacccccgacgatctggagctgcctcgcgtgaccttcatgatggtgaaggacatcgtgatgatcgccttcggcgtgctggccgccatgcagaagaacgtgaccctgaagtggatctttaatctggcctcttacctggtgtgcttctggctggtggtggatctgatccagctgatgaatcagaagaagaagtattttcaggtgcacgagcggtgttgggagtggattctggccacaatgatctacttctttttcgcccagtctctgtttatcttcctgtatgccgtgggccccccttgttttaacatcatgggaagggacggcgataggatcggacacagcgtgggcgacctgtttgccaagaatctgttcggctttttcgcctggtacgtgaggttcgtggtgctggacccctatgccaagcggatcagaggagca

SEQ ID NO: 14 - DNA, 810 bp, Artificial Sequence
  Synthetic polynucleotide
atggaccccctgaacatggacgagcacgaggcctggcgccgcgacttcgcccgcctgctgcaggagcagcccgagagcagcgtgccccagggcgtggccggcgcccgcctgtgcgtgttcagctgcagcatcgccctgatctgcttctacggcgtgcagtaccgcgccggccgctgcaacctgcaccccctgtacgtgcagttcgtgaccttctgcgtgtacagcctgagctgcttcggcaccaccgacttcctgtacatccccatccagaaccacgccggcgagaccttcgacctggccgtgggccgctacctgttctggatgatgacctgccccgtgatcatcagcaacgtgaacatcctgctgaacctgatgacccccgacgacctggagctgccccgcgtgaccttcatgatggtgaaggacatcgtgatgatcgccttcggcgtgctggccgccatgcagaagaacgtgaccctgaagtggatcttcaacctggccagctacctggtgtgcttctggctggtggtggacctgatccagctgatgaaccagaagaagaagtacttccaggtgcacgagcgctgctgggagtggatcctggccaccatgatctacttcttcttcgcccagagcctgttcatcttcctgtacgccgtgggccccccctgcttcaacatcatgggccgcgacggcgaccgcatcggccacagcgtgggcgacctgttcgccaagaacctgttcggcttcttcgcctggtacgtgcgcttcgtggtgctggacccctacgccaagcgcatccgcggcgcc

SEQ ID NO: 15 - DNA, 810 bp, Artificial Sequence
  Synthetic polynucleotide
atggaccccctgaacatggacgagcacgaggcctggagaagagacttcgccagactgctgcaggagcagcccgagagcagcgtgccccagggcgtggccggcgccagactgtgcgtgttcagctgcagcatcgccctgatctgcttctacggcgtgcagtacagagccggcagatgcaacctgcaccccctgtacgtgcagttcgtgaccttctgcgtgtacagcctgagctgcttcggcaccaccgacttcctgtacatccccatccagaaccacgccggcgagaccttcgacctggccgtgggcagatacctgttctggatgatgacctgccccgtgatcatcagcaacgtgaacatcctgctgaacctgatgacccccgacgacctggagctgcccagagtgaccttcatgatggtgaaggacatcgtgatgatcgccttcggcgtgctggccgccatgcagaagaacgtgaccctgaagtggatcttcaacctggccagctacctggtgtgcttctggctggtggtggacctgatccagctgatgaaccagaagaagaagtacttccaggtgcacgagagatgctgggagtggatcctggccaccatgatctacttcttcttcgcccagagcctgttcatcttcctgtacgccgtgggccccccctgcttcaacatcatgggcagagacggcgacagaatcggccacagcgtgggcgacctgttcgccaagaacctgttcggcttcttcgcctggtacgtgagattcgtggtgctggacccctacgccaagagaatcagaggcgcc

SEQ ID NO: 16 - protein, 270 aa, Thraustochytrium sp. ATCC 26185
MDPLNMDEHEAWRRDFARLLQEQPESSVPQGVAGARLCVFSCSIALICFYGVQYRAGRCNLHPLYVQFVTFCVYSLSCFGTTDFLYIPIQNHAGETFDLAVGRYLFWMMTCPVIISNVNILLNLMTPDDLELPRVTFMMVKDIVMIAFGVLAAMQKNVTLKWIFNLASYLVCFWLVVDLIQLMNQKKKYFQVHERCWEWILATMIYFFFAQSLFIFLYAVGPPCFNIMGRDGDRIGHSVGDLFAKNLFGFFAWYVRFVVLDPYAKRIRGA

SEQ ID NO: 17 - protein, 13 aa, Artificial Sequence
  Synthetic polypeptide
GRKKRRQRRRPPQ

SEQ ID NO: 18 - protein, 26 aa, Artificial Sequence
  Synthetic polypeptide
GIGAVLKVLTTGLPALISWIKRKRQQ

SEQ ID NO: 19 - protein, 10 aa, Artificial Sequence
  Synthetic polypeptide
TKIESLKEHG

SEQ ID NO: 20 - protein, 10 aa, Artificial Sequence
  Synthetic polypeptide
TQIENLKEKG

SEQ ID NO: 21 - protein, 26 aa, Artificial Sequence
  Synthetic polypeptide
AALEALAEALEALAEALEALAEAAAA

SEQ ID NO: 22 - protein, 25 aa, Artificial Sequence
  Synthetic polypeptide
GLFEAIEGFIENGWEGMIEGWYGCG

SEQ ID NO: 23 - protein, 16 aa, Artificial Sequence
  Synthetic polypeptide
RQIKIWFQNRRMKWKK